(19) United States
(12) Patent Application Publication
(10) Pub. No.: US 2024/0415631 A1
(43) Pub. Date: Dec. 19, 2024

(54) Polymeric Lens With A Central Hole Surrounded By A Darkened Wall

(71) Applicant: Gholam A. Peyman, Sun City, AZ (US)

(72) Inventor: Gholam A. Peyman, Sun City, AZ (US)

(21) Appl. No.: 18/741,753

(22) Filed: Jun. 12, 2024

Related U.S. Application Data
(63) Continuation-in-part of application No. 18/613,081, filed on Mar. 21, 2024.
Application No. 18/613,081 is a continuation-in-part of application No. 18/219,025, filed on Jul. 6, 2023.
Application No. 18/219,025 is a continuation-in-part of application No. 17/171,988, filed on Feb. 9, 2021.
Application No. 17/171,988 is a continuation-in-part of application No. 16/776,453, filed on Jan. 29, 2020, now Pat. No. 11,372,230.
(60) Provisional application No. 63/472,568, filed on Jun. 12, 2023.
(60) Provisional application No. 63/453,606, filed on Mar. 21, 2023.
(60) Provisional application No. 63/358,794, filed on Jul. 6, 2022.
(60) Provisional application No. 63/398,045, filed on Aug. 15, 2022.
(60) Provisional application No. 63/430,054, filed on Dec. 4, 2022.
(60) Provisional application No. 63/458,606, filed on Apr. 11, 2023.
(60) Provisional application No. 62/972,033, filed on Feb. 9, 2020.
(60) Provisional application No. 62/798,132, filed on Jan. 29, 2019.
(60) Provisional application No. 62/895,185, filed on Sep. 3, 2019.

(51) Int. Cl. A61F 2/14; A61F 2/16

(52) U.S. Cl. CPC A61F 2/15; A61F 2/1648; A61F 2002/1681

(57) ABSTRACT

A polymeric lens is disclosed herein. In one or more embodiments, the polymeric lens includes a lens body formed from a polymeric material, the lens body comprising a central pinhole, the central pinhole being surrounded by a permanently darkened wall defining a visual axis, and the lens body being formed by 3-D printing or molding of the lens body from the polymeric material. A 3-D printed pinhole lens structure is also disclosed herein. In one or more embodiments, the 3-D printed pinhole lens structure includes an insertable pinhole body defining a through pinhole of 1-3 mm in diameter with a surrounding darkened wall having a 0.1-1 mm wall thickness, the insertable pinhole body configured to be inserted inside a lens body of an intraocular lens with a central hole of 1-3 mm in diameter, thereby creating an intraocular lens with a central pinhole.

### CROSS-REFERENCE TO RELATED APPLICATIONS

This patent application claims priority to U.S. Provisional Application No. 63/472,568, entitled “Polymeric Lens With A Central Hole Surrounded By A Darkened Wall”, filed on Jun. 12, 2023; and this patent application is a continuation-in-part of application Ser. No. 18/613,081, entitled “Polymeric Transitional Lens With A Central Hole Surrounded By A Darkened Wall”, filed on Mar. 21, 2024; and Ser. No. 18/613,081 claims priority to U.S. Provisional Application No. 63/453,606, entitled “Polymeric Transitional Lens With A Central Hole Surrounded By A Darkened Wall”, filed on Mar. 21, 2023; and Ser. No. 18/613,081 is a continuation-in-part of application Ser. No. 18/219,025, entitled “Tunable Prism For Vision Correction Of A Patient And Other Applications”, filed on Jul. 6, 2023; and Ser. No. 18/219,025 claims priority to U.S. Provisional Application No. 63/358,794, entitled “Tunable Prism For Vision Correction Of A Patient And Other Applications”, filed on Jul. 6, 2022; U.S. Provisional Application No. 63/398,045, entitled “Tunable Prism For Vision Correction Of A Patient And Other Applications”, filed on Aug. 15, 2022; U.S. Provisional Application No. 63/430,054, entitled “Tunable Prism For Vision Correction Of A Patient And Other Applications”, filed on Dec. 4, 2022; and U.S. Provisional Application No. 63/458,606, entitled “Tunable Prism For Vision Correction Of A Patient And Other Applications”, filed on Apr. 11, 2023; and Ser. No. 18/219,025 is a continuation-in-part of application Ser. No. 17/171,988, entitled “Fluidic Glasses For Correcting Refractive Errors Of A Human Or Animal”, filed on Feb. 9, 2021; and Ser. No. 17/171,988 claims priority to U.S. Provisional Application No. 62/972,033, entitled “Fluidic Glasses For Correcting Refractive Errors Of A Human Or Animal”, filed on Feb. 9, 2020; and Ser. No. 17/171,988 is a continuation-in-part of application Ser. No. 16/776,453, entitled “System For Preventing Motion Sickness Resulting From Virtual Reality Or Augmented Reality”, filed Jan. 29, 2020, now U.S. Pat. No. 11,372,230; and Ser. No. 16/776,453 claims priority to U.S. Provisional Application No. 62/798,132, entitled “System For Preventing Motion Sickness Resulting From Virtual Reality”, filed on Jan. 29, 2019 and U.S. Provisional Patent Application No. 62/895,185, entitled “System For Preventing Motion Sickness Resulting From Virtual Reality Or Augmented Reality”, filed on Sep. 3, 2019; the entire contents of each of which are hereby incorporated by reference.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### INCORPORATION BY REFERENCE OF MATERIAL SUBMITTED ON A COMPACT DISK

Not Applicable.

### BACKGROUND OF THE INVENTION

### 1. Field of the Invention

The invention generally relates to a polymeric transitional lens with a central hole surrounded by a darkened wall. More particularly, in accordance with one aspect of the invention, the invention relates to an optical transitional implant and/or progressive glasses with a central hole surrounded by a darkened wall that are used for treating the vision disorders of albino patients. In accordance with another aspect of the invention, the invention generally relates to a tunable prism for vision correction of a patient and other applications, and a fluidic phoropter system.

### 2. Background

All children are born with a number of refractive errors and the majority of them are not detected until the age of one to two years when one eye or the other becomes dominant while the retina of the other eye does not develop and the eye remains “lazy” and may produce a deviation that is called strabismus. If the strabismus is not corrected within month to a year, the neuronal development in the lazy eye suffers by producing amblyopia in that eye.

One of the reasons for the misconception for not correcting refractive error is that the general theory that the retina is not fully developed, and therefore the eyes do not need to be corrected.

However, there has not been a simple means of measuring the eye's refraction from a child who has not learned to communicate.

In the past, the refractive errors are measured by asking a person to differentiate between the sharpness of one image (e.g., a letter), when different lenses are presented to the eye. This procedure is the so-called “subjective measurement” of the visual acuity of the patient. The problem with the subjective refraction has been the inaccuracy of the information that a person communicates to an ophthalmologist or optometrist, etc.

Until now, the refractive errors of newborns are not checked unless the child is nine to twelve months or older where the eye deviation may become visible to the parents.

Similarly, the refractive power of the eyes of animals is not measured unless there has been a need to replace (e.g., a cataract or a damaged crystalline lens) with another synthetic (e.g., acrylic plastic lens). The measurement have been in the majority of situations not precise using a sciascope, or the examiner dials a certain dioptric power in front of a direct ophthalmoscope in front of his own eye while looking inside the eye of the patient until he sees the fundus sharp. However, this accuracy of this examination depends on how often the examiner's eye is corrected to see the near object to start with.

Therefore, there is a need for fluidic glasses for correcting refractive errors of a human or animal. In addition, there is a need for hybrid fluidic glasses for simultaneous far and near stereovision for correcting vision in adults, babies, or animals. In addition, there is a need for a tunable prism for vision correction of a patient, such as for the correction of phoria, and for use in other applications.

Also, in the past, the refractive error of the baby's eye has not gotten sufficient attention. Unfortunately, the refractive error of a child if uncorrected leads to developmental delay in vision and potentially the brain. Therefore, in general all children should have an eye examination by the age of 6 months or earlier to recognize the general optical deviations, such as farsightedness, astigmatism, and nearsightedness. Babies that are younger than 6 months have a very short focal point of 6-10 inches in front of their eyes. If the eyes have turned from the parallel position or the pupil has lost its dark appearance, the eye needs to be examined to rule out other important ocular pathologies such as presence of a tumor or refractive error of one or both eyes, etc. As the eye grows from age 2 to age 6 to age 14 years the eye become larger and often develop myopia. It is estimated that 40% of children are myopic. If the myopia progresses beyond-6.00 diopter, and the eye becomes larger in size, it can lead to pathologic myopia later in life, affecting the development of the retina, choroid and the macula with a potential loss of sight.

It is known that excessive reading and indoor activities can enhance the progression of the myopia in children, whereas the outdoor activities and correction of refractive errors can slow down the progression.

Similarly, farsightedness, with the short eye size, or differences in optical power of one eye in children can lead to a deviation of one eye in the form of strabismus.

If strabismus is not corrected, it creates amblyopia where the child is not using one eye, etc. for vision and, in this eye, with time the retina or the fovea does not develop properly, leading to significant loss of sight in the affected eye.

If the problem is recognized early and the treatment done early, the eye recovers. The treatment involves eye patching or closing the good eye and correcting the refractive power of the eyes with glasses, contact lenses, and sometimes with surgery. However, if the correction is delayed the retina loses its function and develops amblyopia or lazy eye.

These issues are more complex in albino children in whom the normal pigmentation of the skin, iris, and retinal pigment epithelium is affected leading to numerous symptoms in early childhood or the entire adult life. These children will have both uncorrected refractive power and severe light sensitivity photophobia, and will avoid light, etc.

Since the albinism affects the children at a very young age, the same treatment concept can be applied to improve the visual perception of the healthy babies and albino children by correcting the refractive errors of their eyes and managing severe photophobia simultaneously.

Albino adults require removal of the cataract which aggravates photophobia and should be managed by cataract surgery to replace the crystalline lens with an appropriate intraocular lens to correct refractive errors, but does nothing to their light sensitivity throughout the day or at night, which causes difficulty driving because of the need to avoid the light from oncoming vehicles, or the difficulty associated with driving through a tunnel which is darker than outside environment, or exiting a tunnel and simultaneously avoiding glare.

Albinism is a genetic disease, that affects the pigmentation of the eye and the skin associated with abnormal melanin metabolism. The tyrosinase test of the hair bulb can be negative or positive for pigmentation and some patients have also low blood platelet counts. These patients typically have light skin, hair, refractive errors, and photophobia. A skin biopsy can demonstrate the genetic of the carrier of albinism in female patients. Other abnormalities found are strabismus, foveal hypoplasia, loss of macular pigment, mottling of the retina, hypopigmented fundus, blue iris, and iris defects seen in transillumination.

In ophthalmology, because of the limitation of animal and human corneas, many end stage corneal diseases become inflamed and scarred, thus preventing the light from reaching the retina, which causes blindness in many developed and developing countries.

One of the earliest attempts to replace the cornea has been by the use of an animal or human cornea. Unfortunately, the animal cornea often is rejected by the host humoral or a cellular bio-incompatibility response to the animal cornea, thereby rejecting the human corneal graft in >10% of human corneas.

In situation that the host cornea is completely diseased and scarred, the limitation of availability of the donor corneal tissue prevents the patient from regaining his or her sight by implantation of the human or animal corneas.

To prevent corneal blindness, some attempts have been made to replace the entire cornea with synthetic transparent polymers, such as acrylic or methacrylic polymers, etc., but these procedures have not been 100% successful.

The first full-thickness cornea transplants that have been partially successful are Boston KPro, and Moscow Eye Microsurgery Complex in Russia (MICOF) keratoprostheses, and the osteo-odonto-keratoprosthesis (OOKP), or recent Gore prosthetic cornea.

Unfortunately most of these patients are prone to infection, glaucoma, and the final refractive error of these eyes could not be corrected. This creates a one-size-fits-all limitation for all synthetic transplanted corneas. This means that the eye initially is corrected with the potential corneal refraction that the operator chose for replacement of the corneas.

Even if for a period of time, the keratoprosthesis implants can be tolerated, their refractive error has never been adjusted and unfortunately a large number of them suffer from glaucoma. Because their intraocular pressure could not be measured by any of the available technology since the synthetic corneas are mostly rigid or semi-rigid and the measurement of the eye pressure through them has not been possible leading to unrecognized slow rise of the intraocular pressure, the so-called post prosthetic glaucoma or glaucoma-induced blindness, even if the central part if the synthetic cornea remains transparent.

Therefore, there is a need for techniques, which can be used either with an animal cornea or synthetic acrylic cornea, that are able to modify the refractive error of the implanted eyes after implantation of the (synthetic or non-synthetic keratoprosthesis), and prevent rejection of the synthetic or animal cornea, while, in some cases, also controlling the intraocular pressure by implantation of a device that automatically controls the intraocular pressure after keratoprosthesis.

Therefore, there is further a need for techniques that modify a human or animal cornea to provide better vision for the patient than what has been previously available.

Moreover, motion sickness can be induced by an involuntary motion of the body and the eye. The retinal photoreceptors sense the visual light stimuli induced by the motion in the surrounding environment, which are transmitted as electrical pulses to the brain via the optic nerve. The perception of the body and head location and their motion are perceived by the three semicircular fluid-filled canals and their hair-like sensors stimulated by small stones, which are located in the inner ear and build a part of the vestibular system, connected to the brain through the 8th cranial nerve.

Motion sensation can be felt both by the eye and the vestibular system, or separately. If the signals reach the brain in a coordinated logical format, the brain accepts it as expected or normal consequence of motion. If the sensations are not felt together, the brain may not be able to register it as expected or normal, which can result in a confusion producing the symptoms of motion sickness, e.g., dizziness, imbalance, stiffness of the neck muscles, vertigo, and vomiting, etc.

Virtual reality (VR) is a new computer-generated reality presented to the viewer via a headset and two goggles having two fixed plus lenses inside a viewing box with a semitransparent glass or plastic to exclude the outside world, while immersing the viewer in a separate artificial environment or a combination of virtual and augmented reality (AR). The eyes are in general in an extreme convergent position for near vision to see the images presented to them stereoscopically or in three dimensions.

While about 50% of the adult users may not have any side effects when using the VR goggles, or AR glasses, a large portion of the population will suffer from minor to major discomfort, involving eye strain, dizziness, or imbalance that makes using these units problematic after short or long term use. Often a mismatch between these sensations creates discomfort that can be minor strain of the eye to severe symptoms of dizziness, imbalance, and vomiting, etc.

At present, there is no space for correction of the visual acuity of the person in the headset for the viewer to use his or her daily worn glasses, nor there is any means of correcting the positive or negative or astigmatic dioptric errors of the eyes of the viewer in the relaxed stage or during observation of an object close to the eye that creates a state of accommodation. In this situation, the eyes automatically converge and the ciliary body contracts to create a crystalline lens in the eye which is more convex with a focal point of about 33 cm from the eye. However the closer the object is to the eye the more dioptric power is needed to bring the object or image in the focal point of the eyes.

At present, all VR or AR systems use solid lenses made of solid glass and their power is not adjustable. Only the position of the lenses can be moved closer or further apart to each other to bring them closer or further from each other. These lenses are not automatically corrected for the individuals using them.

As mentioned above, the VR headset is equipped with two sets of plus lenses, despite the statement by the manufacturers that these lenses are adjustable, this statement is related to the position of the lenses or inter-pupillary distance between the eyes, and not to the refractive power of the lenses. This means that all refractive errors of the eye including myopic, hyperopic or astigmatic errors of the eyes remain uncorrected during the use of the VR or AR. In such a situation, the eyes have to fuse the images of two eyes, in the presence of these disparities. This creates eye strain and confusion for the eye and brain. Because the degree of accommodation and convergence differ in each person and with age, these discrepancies alone enhance the potential side effects described and contribute to non-tolerance of the VR headsets. Furthermore, the solid lenses do not provide a means of increasing or decreasing their refractive power, i.e., changing their focal point as the eyes look at an object near or in the far. The simple corrective glasses also cannot adjust themselves to eliminate this problem because their corrective powers are not tunable because the lenses do not change their shape depending on the dioptric power needed in front of the eyes. And they are made to be static (solid lens) for either emmetropic correction of the eye, for the far, at a fixed distance from the eye, or for reading at a distance of about 33 cm from the eyes, etc.

Hereinafter, in this application, solutions to some of the above described problems will be presented. These solutions will make it possible to reduce some of the side effects described above, though there will be always some people who will have difficulty getting used to these side effects, which can be compared to the fear of height.

Furthermore, conventional cameras are known that require the users thereof to manually adjust the focus of a lens prior to taking a photograph so that the acquired image is in-focus. The manual adjustment of the camera lens is laborious and often inaccurate. Thus, what is needed is an automated camera system that comprises means for automatically focusing the camera without the necessity for manual adjustment by the user thereof, and without the need for moving parts on the camera itself. In particular, there is a need for a light field camera with automatic focal point adjustment.

### BRIEF SUMMARY OF EMBODIMENTS OF THE INVENTION

Accordingly, the present invention is directed to a polymeric lens that substantially obviates one or more problems resulting from the limitations and deficiencies of the related art.

In accordance with one or more embodiments of the present invention, there is provided a polymeric lens that includes a lens body formed from a polymeric material, the lens body comprising a central pinhole, the central pinhole being surrounded by a permanently darkened wall defining a visual axis, and the polymeric material outside the central pinhole comprising at least one light-activated, uniformly distributed chromophore that results in a darkening of the lens body outside the central pinhole when the chromophore is activated by light.

In a further embodiment of the present invention, the lens body is configured to be inserted into an eye of a patient to function as an intraocular lens.

In yet a further embodiment, the lens body is configured for use in glasses, as a contact lens, or as an intraocular lens that is capable of being used in an albino person to significantly reduce glare.

In still a further embodiment, the central pinhole of the lens body is in a form of a central aperture disposed through the lens body, and the central aperture extending from an anterior surface of the lens body to a posterior surface of the lens body.

In yet a further embodiment, the central pinhole of the lens body is in a form of a central virtual pinhole formed by a mask that defines the permanently darkened wall of the central virtual pinhole.

In still a further embodiment, the lens body is configured as a refractive lens.

In yet a further embodiment, the lens body is configured as a diffractive or multifocal lens.

In still a further embodiment, the lens body is configured as a non-refractive lens and/or is disposed in cosmetic glasses.

In yet a further embodiment, the lens body is used in glasses or as a contact lens to compensate simultaneously for myopia, farsightedness, and/or astigmatism in children or albino patients so as to enable them to focus for far and near distances with both eyes, thereby preventing development of amblyopia or lazy eye.

In still a further embodiment, the polymeric lens provides the ability to see objects in-focus so as to stabilize the normal growth of the eye, thereby contributing to lowering the incidence of high myopia with its subsequent negative effect on the vision.

In yet a further embodiment, the chromophore in the polymeric material absorbs only the visible light while permitting the infrared light or another selected color of light to pass through.

In still a further embodiment, the polymeric lens is in a form of a collamer lens comprising a combination of collagen and a polymer with a uniformly distributed chromophore that results in a darkening of the lens body outside the central pinhole when the chromophore is activated by light when the polymeric lens is implanted in front of an existing crystalline lens in albinos, or when the polymeric lens is used to replace the existing crystalline lens if the existing crystalline lens is damaged as a result of trauma or aging.

In yet a further embodiment, the central pinhole with the darkened wall has a diameter of at least 1 mm.

In still a further embodiment, the central pinhole with the darkened wall has a diameter that is up to 2 mm or more than 2 mm.

In yet a further embodiment, the central pinhole with the darkened wall has a diameter that is up to 3 mm.

In still a further embodiment, the central pinhole with the darkened wall has a diameter that is up to 4 mm.

In yet a further embodiment, the central pinhole with the darkened wall is in a form of an elongated slit with a width of approximately 1-3 mm and length of approximately 1-5 mm.

In still a further embodiment, the central pinhole of the lens body is in a form of a central virtual pinhole, and the lens body is disposed in glasses with or without refractive power to avoid glare for children and albino patients and permit the light pass through the virtual central pinhole to create an in-focus image on the retina without causing severe glare; and the peripheral part of the lens body outside the central pinhole has the distributed chromophores so as to darken the peripheral part when activated by light, and the central virtual pinhole comprises glass that is made with ultraviolet absorbers to prevent ultraviolet light passing through the central virtual pinhole or a periphery thereof.

In yet a further embodiment, the lens body is made from two separate parts that comprise a central part and a peripheral part, the central part is a circular part of 1-3 mm in diameter in a form of an ultraviolet absorbing lens that is glued within a circular hole of the peripheral part with light-activated molecules, and both the central part and the peripheral part are glued with the back central dark mask using a glue on one side or both sides of the lens body, thereby creating a virtual central pinhole that absorbs only UV light, while the peripheral part of the lens has the light-activated molecules that darken in color depending on the intensity of the light.

In still a further embodiment, the peripheral part of the lens is configured as a refractive lens to correct refractive errors of a patient.

In yet a further embodiment, the peripheral part of the lens is configured as a cosmetic lens portion without refractive correction.

In still a further embodiment, the chromophore is selected from the group consisting of a compound that selectively absorbs ultraviolet light, a compound that selectively absorbs visible light, a compound that polarizes light, and combinations thereof.

In yet a further embodiment, the lens body is configured as a refractive lens to reduce a glare sensation, while correcting a refractive error in albinos and children.

In still a further embodiment, the lens body is disposed in a frame.

In yet a further embodiment, the frame comprises at least one light-activated chromophore.

In accordance with one or more other embodiments of the present invention, there is provided an intraocular polymeric lens that includes a lens body formed from a polymeric material, the lens body comprising a central pinhole, the central pinhole being in a form of a horizontal oval slit surrounded by a permanently darkened wall defining a visual axis, and the polymeric material outside the central pinhole comprising at least one light-activated, uniformly distributed chromophore that results in a darkening of the lens body outside the central pinhole when the chromophore is activated by light. In these one or more other embodiments, after production, the lens body is encapsulated in a non-permeable thin layer of carbon by electron spattering, plasma spattering, or nanotechnology transparent molecules that form a transparent capsule to retain the chromophore permanently inside the intraocular polymeric lens.

In accordance with yet one or more other embodiments of the present invention, there is provided a polymeric lens that includes a lens body formed from a polymeric material, the lens body comprising a central pinhole, the central pinhole being surrounded by a permanently darkened wall defining a visual axis, and the polymeric material outside the central pinhole comprising at least one light-activated, uniformly distributed chromophore that results in a darkening of the lens body outside the central pinhole when the chromophore is activated by light. In these one or more other embodiments, the permanently darkened wall surrounding the pinhole is formed by adding a dark material to the lens body while the polymeric lens is being molded or 3D printed, thereby leaving a central area transparent polymeric area with an ultraviolet absorbing dye forming the central pinhole so as to produce a transitional lens in combination with a virtual central pinhole.

In a further embodiment of the present invention, the lens body is configured for use in glasses, as a contact lens, a scleral lens, or as an intraocular lens that is capable of being used in an albino person to significantly reduce glare.

In accordance with yet one or more other embodiments of the present invention, there is provided a polymeric lens that includes a lens body formed from a polymeric material, the lens body comprising a central pinhole, the central pinhole being surrounded by a permanently darkened wall defining a visual axis, and the lens body being formed by 3-D printing or molding of the lens body from the polymeric material.

In a further embodiment of the present invention, the polymeric lens further comprises one or more haptics attached to the lens body, the lens body being configured for use as an intraocular lens, and the lens body being configured to be positioned between a posterior iris and an anterior lens capsule of an eye.

In yet a further embodiment, the permanently darkened wall is made using the same polymeric material as the surrounding portion of the lens body, the permanently darkened wall being formed by adding a darkening compound to the polymeric material while 3-D printing the polymeric lens.

In still a further embodiment, the darkening compound used to form the permanently darkened wall in the lens body comprises carbon nanoparticles

In yet a further embodiment, the central pinhole of the lens body is in a form of a central virtual pinhole formed from the polymeric material without the darkening compound added thereto so as to form a central transparent virtual pinhole without the need for an external mask.

In still a further embodiment, the lens body with the central virtual pinhole is configured for use in glasses, as a contact lens, as a scleral lens, or as an intraocular lens that is capable of being used to significantly reduce glare for any person particularly sensitive to glare.

In yet a further embodiment, a darkened central zone of the polymeric lens is formed by adding a darkening compound to the polymeric material while 3-D printing the lens body so as to form the lens body with the darkened central zone and a transparent peripheral zone surrounding the lens body, and wherein the darkened central zone of the lens body is subsequently drilled so as to form the central pinhole with the surrounding permanently darkened wall.

In still a further embodiment, the polymeric material outside the central pinhole comprises at least one light-activated, uniformly distributed chromophore that results in a darkening of the lens body outside the central pinhole when the chromophore is activated by light.

In yet a further embodiment, the lens body with the central pinhole is configured for use in glasses, as a contact lens in an eye of a patient, as a scleral lens in an eye of a patient, as an intraocular lens in an eye of a patient, as a camera lens in a camera or fluidic camera, as a telescope lens in a machine vision telescope, in a virtual reality headset, or in an augmented reality headset.

In accordance with still one or more other embodiments of the present invention, there is provided a 3-D printed pinhole lens structure that includes an insertable pinhole body defining a through pinhole of 1-3 mm in diameter with a surrounding darkened wall having a 0.1-1 mm wall thickness, the insertable pinhole body configured to be inserted inside a lens body of an intraocular lens with a central hole of 1-3 mm in diameter, thereby creating an intraocular lens with a central pinhole.

In a further embodiment of the present invention, after the insertable pinhole body is formed by 3-D printing, the through pinhole in the insertable pinhole body is formed by drilling a through hole through the insertable pinhole body.

In yet a further embodiment, the insertable pinhole body comprises a lower cylindrical body portion with a top peripheral flange portion connected to the lower cylindrical body portion.

Additional features and advantages are described herein, and will be apparent from, the following Detailed Description and the figures.

It is to be understood that the foregoing general description and the following detailed description of the present invention are merely exemplary and explanatory in nature. As such, the foregoing general description and the following detailed description of the invention should not be construed to limit the scope of the appended claims in any sense.

### BRIEF DESCRIPTION OF THE SEVERAL VIEWS OF THE DRAWINGS

The invention will now be described, by way of example, with reference to the accompanying drawings, in which:

FIG. 1 is a perspective view of a person wearing a virtual reality headset that includes fluidic lenses in the headset for correcting the refractive errors of the person, according to one embodiment of the invention;

FIG. 2 is a diagrammatic representation of the refractive error correction system utilized in conjunction with the fluidic lenses disposed in the virtual reality headset of FIG. 1;

FIG. 3 is a detail view of the pump and servomotor that is used in the refractive error correction system of FIG. 2;

FIG. 4A is a diagrammatic view of a system that utilizes a plurality of prisms to separate the images of each eye to reduce muscular fatigue during convergence, according to another embodiment of the invention;

FIG. 4B is a diagrammatic view of a system where the images that are projected onto a screen are seen by each eye separately, according to yet another embodiment of the invention;

FIG. 4C is a top view of a spherical fluidic lens and two astigmatic cylindrical fluidic lenses that may be included in the system of FIG. 4B for each eye;

FIG. 4D depicts top views of pinpoint transitional lenses for correcting refractive power for any distance, according to still another embodiment of the invention;

FIG. 4E depicts side views of the pinpoint transitional lenses of FIG. 4D;

FIG. 5A is a top view of a first transparent circular plate used for correcting a convergence problem associated with a user of a virtual reality (VR) headset, according to still another embodiment of the invention;

FIG. 5B is a top view of a second transparent circular plate used for correcting a convergence problem associated with a user of a virtual reality (VR) headset;

FIG. 5C is a top view of two transparent circular plates used for correcting a convergence problem associated with a user of a virtual reality (VR) headset, wherein the upper transparent circular plate has a magnetic material at the peripheral edge thereof, and the lower transparent circular plate has a series of activatable electromagnets at the peripheral edge thereof, according to yet another embodiment of the invention;

FIG. 5D is a top view of a transparent balloon (or a ball-shaped flexible transparent polymer) that is able to be filled with a fluid for creating a tunable prism (or when it is in the form of a ball, it does not need to be filled because it is made of a flexible or semisolid transparent polymer with no cavity), according to still another embodiment of the invention;

FIG. 5E is a side view of a tunable prism in a parallel configuration, where the tunable prism utilizes the transparent balloon or ball of FIG. 5D;

FIG. 5F is a side view of the tunable prism of FIG. 5E in a first tilted configuration;

FIG. 5G is a side view of a tunable prism of FIG. 5E in a second tilted configuration;

FIG. 5H is a side view of another tunable prism, wherein the bottom plate of the tunable prism comprises a central hole formed therein, according to yet another embodiment of the invention;

FIG. 5I illustrates varying degrees of activation of a tunable prism, which is similar to the tunable prism depicted in FIGS. 5E, 5F, and 5G;

FIG. 5J is another side view of the tunable prism of FIG. 5H, wherein the balloon of the tunable prism is shown protruding through the central hole in the bottom plate, and the balloon is compressed using magnets without the use of a pump;

FIG. 6A is a side view of another tunable prism, wherein a pump is shown connected to the balloon of the tunable prism for inflating the balloon so as to create a convex lens, according to still another embodiment of the invention;

FIG. 6B is a side view of a combined prism and tunable lens, wherein the combined prism and tunable lens (e.g., a liquid lens) is provided with both a pump and magnetic actuation means, according to yet another embodiment of the invention;

FIG. 6C is a top view of the back plate of the combined prism and tunable lens illustrated in FIG. 6B, wherein the circular opening in the back plate is depicted;

FIG. 6D is a side view of a combined prism and tunable lens similar to that of FIG. 6B, except that the back plate of the combined prism and tunable lens is provided with a rectangular opening rather than a circular opening for creation of an astigmatic tunable lens, according to still another embodiment of the invention;

FIG. 6E is a top view of the back plate of the combined prism and tunable flexible lens illustrated in FIG. 6D, wherein the rectangular opening in the back plate is depicted;

FIG. 6F is a top view of a universal prism created by two separate liquid or tunable lenses located 45 degrees from each other, according to yet another embodiment of the invention;

FIG. 6G is a top view of another universal prism and astigmatic lenses that is created by two prismatic lenses located 45 degrees from each other, according to still another embodiment of the invention;

FIG. 7A is a side view of another tunable prism, wherein a spring coil is used in the prism rather than a balloon or ball, according to yet another embodiment of the invention; and

FIG. 7B is a side view of still another embodiment of a tunable prism, wherein the tunable prism utilizes a combination of a spring coil and a balloon or ball made of a transparent polymer, such as silicone, etc.

FIG. 8 is a side sectional view of a fluidic lens with a flexible membrane in a convex configuration for correction of farsightedness of an eye (hyperopia), according to an illustrative embodiment of the invention;

FIG. 9 is a side sectional view of a fluidic lens with a flexible membrane in a concave configuration for correction of nearsightedness of an eye (i.e., myopia), according to an illustrative embodiment of the invention;

FIG. 10 is a side sectional view of a presbyopic bifocal fluidic lens with two fluidic chambers for correcting for both hyperopia and myopia, according to an illustrative embodiment of the invention;

FIG. 11 is a side sectional view of the presbyopic bifocal fluidic lens of FIG. 10, where the front fluidic lens is disposed in a concave configuration for correction of myopia, and the rear fluidic lens is disposed in a convex configuration for correction of hyperopia, according to an illustrative embodiment of the invention;

FIG. 12 is a side sectional view of a diffractive fluidic lens having a front fluidic lens chamber with a flexible membrane and a rear Fresnel diffractive lens with multiple zones of prisms to provide many fixed diffractive plus zone focal points, according to an illustrative embodiment of the invention;

FIG. 13 is a top view of the diffractive fluidic lens of FIG. 12;

FIG. 14A is a top view of a back plate of a fluidic chamber of a fluidic lens, where the back plate is in the form of a transitional lens with a pigment that changes color based upon the amount of light absorbed, according to an illustrative embodiment of the invention;

FIG. 14B is a top view of another back plate of a fluidic chamber of a fluidic lens, where the back plate is in the form of a transitional lens in which the pigment does not cover a small central area of the plate, thereby creating a pinhole configuration in the plate when the plate is exposed to light, according to an illustrative embodiment of the invention;

FIG. 14C is a top view of yet another back plate of a fluidic chamber of a fluidic lens, where the back plate is in the form of a diffractive lens in which the central area of the plate is not diffractive, thereby creating a pinhole configuration in the plate, according to an illustrative embodiment of the invention;

FIG. 14D is a top view of still another back plate of a fluidic chamber of a fluidic lens, where the back plate is in the form of a diffractive transitional lens in which the pigment does not cover a small non-diffractive central area of the plate, thereby creating a pinhole configuration in the plate when the plate is exposed to light, according to an illustrative embodiment of the invention;

FIG. 15 is a perspective view depicting fluidic adjustable glasses disposed on a person, the fluidic adjustable glasses including one or more lenses illustrated in FIGS. 8-14D, according to an illustrative embodiment of the invention;

FIG. 16A is a side view of a hybrid fluidic lens with one or more transparent plates, where the rear transparent plate is diffractive and may be provided with a transitional pigment, according to an illustrative embodiment of the invention;

FIG. 16B is a top view of the diffractive fluidic lens of FIG. 16A;

FIG. 17 is a side view of a hybrid fluidic lens similar to that of FIGS. 8 and 9, except that the back plate of the hybrid fluidic lens has an additional plus lens for presbyopia correction;

FIG. 18 is a front view of eyes of a patient without any phoria condition;

FIG. 19 is a front view of eyes of a patient having a phoria condition resulting in a deviation of 1 millimeter (mm) in the left eye of the patient;

FIG. 20 is a front view of eyes of a patient having a phoria condition resulting in a deviation of 2 millimeters (mm) in the left eye of the patient;

FIG. 21 is a front view of eyes of a patient having a phoria condition resulting in a deviation of 3 millimeters (mm) in the left eye of the patient;

FIG. 22 is a front view of a left eye of a patient having a hyperphoria condition;

FIG. 23 is a front view of a left eye of a patient having a hypophoria condition;

FIG. 24 is a front view of a left eye of a patient having an oblique hyperphoria condition;

FIG. 25 is a front view of a left eye of a patient having an exophoria condition;

FIG. 26 is a front view of a left eye of a patient having an oblique hypophoria condition;

FIG. 27 is a side view of a tunable prism in a parallel configuration, where the tunable prism utilizes a deformable balloon or ball with or without a tube;

FIG. 28 is a side view of a pair of tunable prisms in a first vision correction configuration, where the tunable prisms are magnetically activated so as to create a base-in prism;

FIG. 29 is a side view of a pair of tunable prisms in a second vision correction configuration, where the tunable prisms are magnetically activated so as to create a base-out prism;

FIG. 30 is a side view of a pair of vertically activated tunable prisms, where each of the tunable prisms is magnetically activated in different vision correction configurations;

FIG. 31 is a diagrammatic representation of a home monitoring system for evaluating a refractive error and/or an ocular disease of a patient, wherein the home monitoring system includes a fluidic phoropter, a camera to photograph the retina, and a remote Shack Hartmann sensor connected via the cloud;

FIG. 32 is a diagrammatic representation of a home monitoring system for evaluating a refractive error and/or an ocular disease of a patient, wherein the home monitoring system includes a fluidic phoropter, and a remote Shack Hartmann sensor with artificial intelligence (AI) software connected via the cloud;

FIG. 33 is a diagrammatic representation of AR or VR goggles with tunable prisms, fluidic lenses, and electronics that are connected via the cloud to a remote Shack Hartmann sensor with artificial intelligence (AI) software for binocular vision, imaging of the cornea, and/or diagnosis of a disease of a wearer of the AR or VR goggles;

FIG. 34A is a side view of an illustrative embodiment of a tunable prism in a parallel configuration, where the tunable prism utilizes a transparent flexible ball;

FIG. 34B is a side view of the tunable prism of FIG. 34A in a first tilted configuration;

FIG. 34C is a side view of the tunable prism of FIG. 34A in a second tilted configuration;

FIG. 35A is a side view of an illustrative embodiment of a tunable prism in a parallel configuration, where the top transparent plate has a convex upper surface;

FIG. 35B is a side view of the tunable prism of FIG. 35A in a first tilted configuration;

FIG. 35C is a side view of the tunable prism of FIG. 35A in a second tilted configuration;

FIG. 36A is a side view of an illustrative embodiment of a tunable prism in a parallel configuration, where the top transparent plate has a concave upper surface;

FIG. 36B is a side view of the tunable prism of FIG. 36A in a first tilted configuration;

FIG. 36C is a side view of the tunable prism of FIG. 36A in a second tilted configuration;

FIG. 36D is another side view of the tunable prism of FIG. 36A in a parallel configuration, where the transparent flexible ball between the plates has been compressed;

FIG. 37A is a side view of an illustrative embodiment of a tunable prism in a parallel configuration, where the top transparent plate has an opening through which a flexible balloon or ball is able to bulge out;

FIG. 37B is a side view of the tunable prism of FIG. 37A in a first tilted configuration;

FIG. 37C is a side view of the tunable prism of FIG. 37A in a second tilted configuration;

FIG. 38A is a side view of an illustrative embodiment of a tunable prism in a parallel configuration, where the top transparent plate has a diffractive upper surface and the tunable prism is magnetically actuated;

FIG. 38B is a side view of the tunable prism of FIG. 38A in a tilted configuration after the tunable prism has been magnetically actuated;

FIG. 39A is a side view of an illustrative embodiment of a tunable prism in a first tilted configuration, where a pump is shown connected to an inflatable balloon of the tunable prism for inflating the balloon, and the top and bottom transparent plates are connected to one another by a joint or hinge;

FIG. 39B is a side view of the tunable prism of FIG. 39A in a second tilted configuration, where the balloon of the tunable prism in FIG. 39B is more inflated than that which is depicted in FIG. 39A;

FIG. 40 is a side view of an illustrative embodiment of a tunable prism in a tilted configuration, where a pump is shown connected to an inflatable balloon of the tunable prism for inflating the balloon, the top and bottom transparent plates are connected to one another by a joint or hinge, and the top transparent plate has a Fresnel prism-like or diffractive upper surface;

FIG. 41A is a front view of a transitional lens containing chromophores and a central pinhole passing through the lens body that is surrounded by a darkened wall, according to another illustrative embodiment of the invention;

FIG. 41B is a front view of a transitional lens with chromophores and a mask defining a virtual pinhole with no aperture passing through the lens body, according to yet another illustrative embodiment of the invention;

FIG. 41C is a side cross-sectional view through the transitional lens of FIG. 41A, which shows the permanently darkened wall of the pinhole;

FIG. 41D is a side cross-sectional view through the transitional lens of FIG. 41B, which shows the mask defining the virtual pinhole;

FIG. 41E is a front view of a transitional lens containing chromophores and a central slit-shaped pinhole passing through the lens body that is surrounded by a darkened wall, according to still another illustrative embodiment of the invention;

FIG. 42A is a side cross-sectional view through the transitional lens of FIG. 41A, wherein the transitional lens is depicted in a low level lighting condition where the chromophores in the lens body are not activated such that light passes through the lens body outside of the pinhole;

FIG. 42B is a side cross-sectional view through the transitional lens of FIG. 41A, wherein the transitional lens is depicted in a high level lighting condition where the chromophores in the lens body are activated such that light is prevented from passing through the lens body outside of the pinhole;

FIG. 42C is a side cross-sectional view through a transitional lens that is similar to the transitional lens of FIGS. 41B and 41D, according to yet another illustrative embodiment of the invention, wherein a mask defines a virtual pinhole with no aperture passing through the lens body;

FIG. 42D is another side cross-sectional view of the transitional lens of FIG. 42C, wherein the transitional lens is depicted in a high level lighting condition where the chromophores in the lens body are activated such that light is prevented from passing through the lens body outside of the pinhole;

FIG. 42E is another side cross-sectional view of the transitional lens of FIG. 42C, wherein the transitional lens is depicted in a low level lighting condition where the chromophores in the lens body are not activated such that light passes through the lens body outside of the pinhole;

FIG. 43A is a front view of a transitional lens containing chromophores where the central circular part is about to be cut away to form a central hole in the lens body, according to still another illustrative embodiment of the invention;

FIG. 43B is another front view of the transitional lens of FIG. 43A, where the central circular part has been cut away so as to form a central hole in the lens body that is surrounded by a peripheral portion of the lens body containing the chromophores for absorbing all wavelengths of light;

FIG. 43C is yet another front view of the transitional lens of FIG. 43A, where a central lens section has been inserted into the central hole in the lens body, the central lens section being formed from a different light absorber material that is sensitive to only ultraviolet (UV) light;

FIG. 43D is a side cross-sectional view through the transitional lens of FIG. 43B, where the central hole in the lens body is depicted;

FIG. 43E is a side cross-sectional view through the transitional lens of FIG. 43C, where central lens section containing the UV light absorber is depicted;

FIG. 44 is a front view of a transitional lens that is similar to the transitional lens of FIG. 43C, according to yet another illustrative embodiment of the invention, where the peripheral portion of the lens body contains the chromophores for absorbing all wavelengths of light, while the central lens section is formed from a different light absorber material that is sensitive to only ultraviolet (UV) light;

FIG. 45 is a front view of a transitional lens that is similar to the transitional lens of FIG. 41B, according to yet another illustrative embodiment of the invention, where the lens body comprises a mask defining a virtual central pinhole with no transitional properties and a peripheral lens body portion surrounding the mask that contains chromophores for operating as a transitional lens;

FIG. 46 is another front view of the transitional lens of FIG. 45, wherein the transitional lens is depicted in a low level lighting condition where the chromophores in the peripheral lens body portion are not activated such that light passes through the lens body outside of the pinhole;

FIG. 47 is yet another front view of the transitional lens of FIG. 45, wherein the transitional lens is depicted in a high level lighting condition where the chromophores in the peripheral lens body portion are activated such that light is prevented from passing through the lens body outside of the pinhole;

FIG. 48 is a front view of a combined transitional and presbyopia lens in a form of a contact lens, according to still another illustrative embodiment of the invention, where the combined transitional and presbyopia lens is provided with a circular through hole or virtual pinhole;

FIG. 49 is a front view of a combined progressive and transitional lens, according to yet another illustrative embodiment of the invention, where the lens body comprises a central pinhole with no transitional properties and a peripheral lens body portion surrounding the mask that contains chromophores for operating as a transitional lens;

FIG. 50A is a front view of another transitional lens with a virtual pinhole, according to still another illustrative embodiment of the invention;

FIG. 50B is a front view of another transitional lens with a central horizontal slit passing through the lens body that is surrounded by a darkened wall, according to yet another illustrative embodiment of the invention;

FIG. 50C is a front view of yet another transitional lens with a central vertical slit passing through the lens body that is surrounded by a darkened wall, according to yet another illustrative embodiment of the invention, wherein the central vertical slit is provided with dumbbell-shaped extensions at both ends of the slit to prevent light scattering;

FIG. 51A is a front view of a 3-D printed lens with a clear peripheral portion and a central dark area where a central circular part is about to be cut away to form a central hole in the lens body, according to still another illustrative embodiment of the invention;

FIG. 51B is another front view of the 3-D printed lens of FIG. 51A, where the central circular part has been cut away so as to form a central hole in the lens body that is surrounded by the clear peripheral portion of the lens body;

FIG. 52 is a front view of a clear 3-D printed lens with plus or minus optical power, according to yet another illustrative embodiment of the invention;

FIG. 53 is a front view of a 3-D printed lens with a clear peripheral portion and a clear central pinhole bounded by a darkened peripheral wall, according to still another illustrative embodiment of the invention;

FIG. 54 is a front view of a 3-D printed lens made of mixed polymers, according to yet another illustrative embodiment of the invention;

FIG. 55 is a front view of a 3-D printed lens with a clear peripheral portion and a clear central pinhole bounded by a darkened peripheral wall, according to still another illustrative embodiment of the invention;

FIG. 56A is a front view of a 3-D printed transitional lens containing chromophores and a central pinhole passing through the lens body that is surrounded by a darkened wall, according to yet another illustrative embodiment of the invention, wherein the transitional lens is depicted in a low level lighting condition where the chromophores in the lens body are not activated such that light passes through the lens body outside of the pinhole;

FIG. 56B is a front view through the 3-D printed transitional lens of FIG. 56A, wherein the transitional lens is depicted in a high level lighting condition where the chromophores in the lens body are activated such that light is prevented from passing through the lens body outside of the pinhole;

FIG. 57 is a side cross-sectional view through an eye of a patient;

FIG. 58 is another side cross-sectional view through the eye of FIG. 57 with a collamer intraocular lens implanted in the eye, according to an illustrative embodiment of the invention;

FIG. 59 is yet another side cross-sectional view through the eye of FIG. 57 with an intraocular lens implanted in the eye in a location anterior of the lens capsule after cataract extraction, wherein the haptics of the intraocular lens extend behind the lens capsule, according to another illustrative embodiment of the invention;

FIG. 60 is still another side cross-sectional view through the eye of FIG. 57 with an intraocular lens implanted inside the lens capsule, according to yet another illustrative embodiment of the invention;

FIG. 61 is yet another side cross-sectional view through the eye of FIG. 57 with an intraocular lens implanted inside the lens capsule, the intraocular lens being 3-D printed with a hole extending through the body of the intraocular lens, according to still another illustrative embodiment of the invention;

FIG. 62 is a front view of a 3-D printed intraocular lens with haptics and a central pinhole surrounded by a darkened peripheral wall, according to yet another illustrative embodiment of the invention;

FIG. 63 is a side perspective view of a 3-D printed insertable pinhole lens structure with a lower cylindrical body portion, a top peripheral flange portion connected to the lower cylindrical body portion, and a central pinhole extending through the pinhole lens structure, according to still another illustrative embodiment of the invention;

FIG. 64 is a front view of a 3-D printed intraocular lens with haptics, a central darkened zone, and a peripheral clear zone surrounding the central darkened zone, according to yet another illustrative embodiment of the invention;

FIG. 65 is another front view of the 3-D printed intraocular lens of FIG. 64 after a central hole has been drilled through the central darkened zone so as to create the through pinhole;

FIG. 66 is a front view of a 3-D printed intraocular lens with a central virtual pinhole and a darkened wall surrounding the central virtual pinhole, according to still another illustrative embodiment of the invention;

FIG. 67 is a front view of a 3-D printed intraocular lens with haptics, a central virtual pinhole, a darkened wall surrounding the central virtual pinhole, and a light-activated, uniformly distributed chromophore in a peripheral portion of the intraocular lens surrounding around the darkened wall, according to yet another illustrative embodiment of the invention;

FIG. 68 depicts a 3-D printed insertable pinhole lens structure and the manner in which the pinhole lens structure is inserted into an intraocular lens, according to still another illustrative embodiment of the invention;

FIG. 69 depicts a one-piece intraocular lens and 3-D printed pinhole lens structure, according to yet another illustrative embodiment of the invention;

FIG. 70 depicts a 3-D printed insertable pinhole lens structure with a top peripheral flange portion formed using a gray polymeric material, according to still another illustrative embodiment of the invention;

FIG. 71 depicts a 3-D printed insertable pinhole lens structure formed using a dark polymeric material where the central pinhole is subsequently drilled therein, according to yet another illustrative embodiment of the invention;

FIG. 72 depicts front and side views of a 3-D printed intraocular lens with haptics, a central virtual pinhole, and a darkened wall surrounding the central virtual pinhole, according to still another illustrative embodiment of the invention;

FIG. 73 is a front view of a 3-D printed intraocular lens with haptics, a central virtual pinhole, a darkened wall surrounding the central virtual pinhole, and a light-activated, uniformly distributed chromophore in a peripheral portion of the intraocular lens surrounding around the darkened wall, according to yet another illustrative embodiment of the invention;

FIG. 74A is a front view of a transitional lens with a lens body having a transparent polymeric central pinhole region and a peripheral transitional region with chromophores, wherein the transitional lens is depicted in a low level lighting condition where the chromophores in the peripheral transitional region of the lens body are not activated such that light passes through the lens body outside of the central pinhole region, according to still another illustrative embodiment of the invention;

FIG. 74B is a front view of the transitional lens of FIG. 74A, wherein the transitional lens is depicted in a high level lighting condition where the chromophores in the peripheral transitional region of the lens body are activated such that light is prevented from passing through the lens body outside of the central pinhole region;

FIG. 75A is a side cross-sectional view of a transitional lens with a lens body having a transparent polymeric central pinhole region and a peripheral transitional region with chromophores, wherein the transitional lens is depicted in a low level lighting condition where the chromophores in the peripheral transitional region of the lens body are not activated such that light passes through the lens body outside of the central pinhole region;

FIG. 75B is a side cross-sectional view of the transitional lens of FIG. 75A, wherein the transitional lens is depicted in a high level lighting condition where the chromophores in the peripheral transitional region of the lens body are activated such that light is prevented from passing through the lens body outside of the central pinhole region;

FIG. 76A is a front view of a transitional lens with a central darkened zone and a transitional peripheral zone surrounding the central darkened zone, according to yet another illustrative embodiment of the invention; and

FIG. 76B is another front view of the transitional lens of FIG. 76A after a central hole has been drilled through the central darkened zone so as to create a through pinhole.

Throughout the figures, the same parts are always denoted using the same reference characters so that, as a general rule, they will only be described once.

### DETAILED DESCRIPTION OF EMBODIMENTS OF THE INVENTION

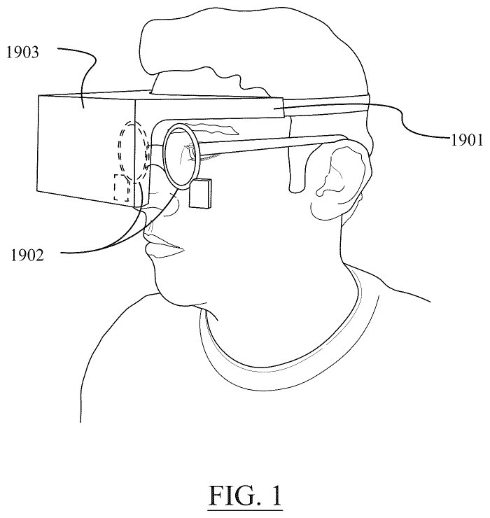

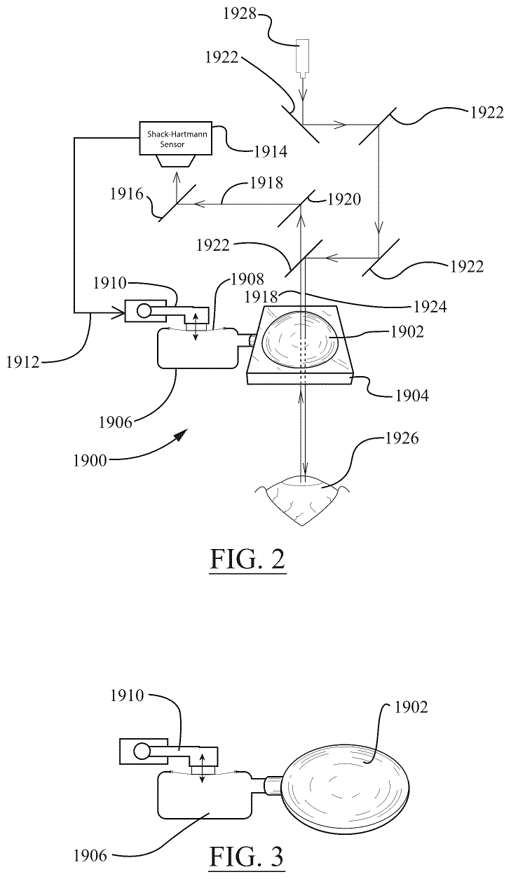

FIGS. 1 and 2 illustrate one embodiment of the present invention. In particular, FIG. 1 illustrates a virtual reality headset 1901 on a person, the virtual reality headset 1901 configured to create an artificial environment and/or immersive environment for the person. As shown in FIG. 1, the virtual reality headset 1901 includes a pair of fluidic lenses 1902 disposed between the respective eyes of the person and a screen 1903 of the virtual reality headset 1901, the fluidic lenses 1902 each having a chamber that receives a fluid therein, and the fluidic lenses 1902 configured to correct the refractive errors of the eyes of the person.

In particular, FIG. 2 illustrates the refractive error correction system 1900 that is utilized in conjunction with the fluidic lenses 1902 disposed in the virtual reality (VR) headset 1901 of FIG. 1. The refractive error correction system 1900 may also be used with an augmented reality (AR) headset. As shown in FIG. 2, the refractive error correction system 1900 generally comprises at least one fluidic lens 1902 disposed between the light source 1928 of the VR or AR headset and the eye 1926 of the person wearing the VR or AR headset; a pump 1906 operatively coupled to the at least one fluidic lens 1902, the pump 1906 configured to insert an amount of the fluid into the chamber of the at least one fluidic lens 1902, or remove an amount of the fluid from the chamber of the at least one fluidic lens 1902, in order to change the shape of at least one fluidic lens 1902 in accordance with the amount of fluid therein; and a Shack-Hartmann sensor assembly 1914 operatively coupled to the pump 1906 via a data processor and control wiring 1912, the Shack-Hartmann sensor assembly 1914 by means of the pump 1906 configured to automatically control the amount of the fluid in the chamber of the at least one fluidic lens 1902, thereby automatically correcting the refractive errors of the eye 1926 of the person wearing the VR or AR headset.

Referring again to FIG. 2, it can be seen that the light emanating from the light source 1928 of the VR or AR headset is diverted around the holographic optical element or diffractive lens 1920 by means of dichroic mirrors or prisms 1922 until the light 1924 enters the cyc 1926 of the person wearing the VR or AR headset. In FIG. 2, it can be seen that a portion 1918 of the light entering the eye 1926 is reflected back from the eye 1926 and initially through the holographic optical element or diffractive lens 1920 and then subsequently through the holographic optical element or diffractive lens 1916 until reaching the Shack-Hartmann sensor assembly 1914. Based on the reflected light 1918, the Shack-Hartmann sensor assembly 1914 controls the action of the servomotor 1910 of the pump 1906, and thus, the amount of fluid that is added to, or removed from, the fluidic lens 1902 automatically.

In the illustrative embodiment of FIG. 2, the fluidic lens 1902 comprises a flexible fluidic membrane that is disposed within a rigid outer housing 1904. The pump 1906 on the illustrative embodiment comprises a pump membrane 1908 that is driven up or down by the servomotor 1910 in order to add or remove fluid from the chamber of the fluidic lens 1902.

A detail view of the pump 1906 that is used in the refractive error correction system 1900 of FIG. 2 is depicted in FIG. 3. In FIG. 3, the servomotor 1910 drives the pump 1906 so as to add or remove fluid from the chamber of the fluidic lens 1902.

In one embodiment, the accommodation of the lenses can be addressed by having a layer of liquid crystal that responds by activating the molecular position of the liquid crystal increasing their index of refraction as needed for near vision under an electrical current.

In one embodiment, the lenses are soft compressive polymeric lenses that can be compressed or decompressed via an electrical pulse to make them more or less convex when protruding through the second plate with a circular hole in it.

In another embodiment, the lenses can be made using a combination of two fluids with different indexes of refraction and their interface can create a positive or negative surface by changing the electrostatic potential between both surfaces using electrical pulses, though they have the shortcoming of not correcting the astigmatic aberrations.

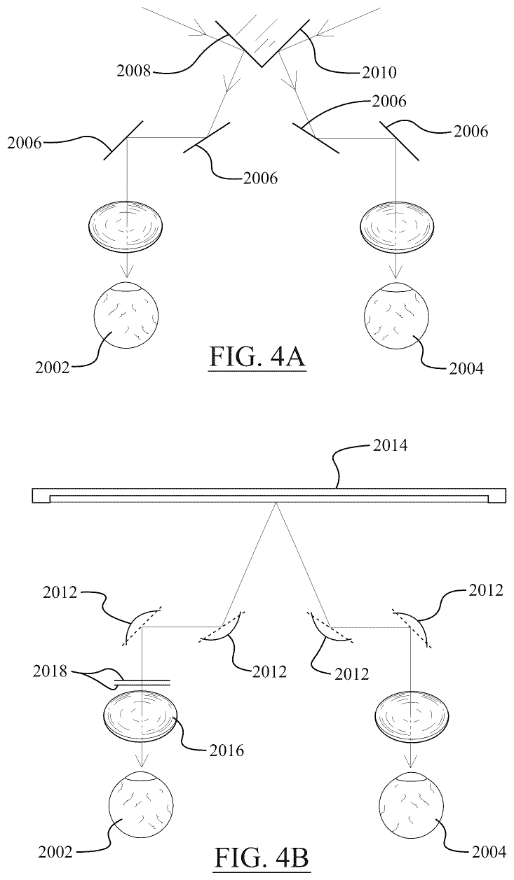

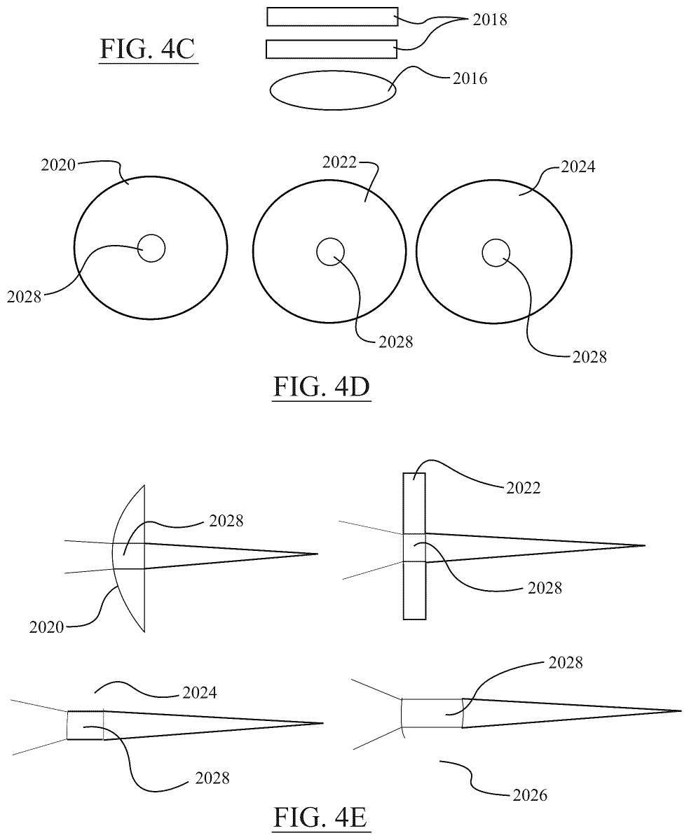

In one embodiment, one can eliminate the problems of muscular fatigue during convergence by separating the images 2008, 2010 of each eye 2002, 2004 using various prisms 2006 as shown in FIG. 4A. In FIG. 4A, the left and right images 2008, 2010 are projected via a series of prisms 2006 that ultimately are perpendicular to either the right or left eye 2002, 2004. In FIG. 4B, the images are projected using various flat or concave prisms 2012 over a screen 2014 that are seen by the eye separately, similar to an IMAX 3-D movie theater. FIG. 4C depicts a spherical fluidic lens 2016 or tunable lens and two astigmatic cylindrical fluidic lenses 2018 that can be adjusted via a pump to correct refractive errors of an eye looking at any distance, and may be used in the system of FIG. 4B. FIGS. 4D and 4E depict an alternative system to FIG. 4C that uses transitional pinhole lenses to correct the refractive power of the lenses for any distance, and the images are projected in the eye perpendicular to the eye's position once the person has the goggles on, regardless of which direction he or she moves his or her head and the refractive error of the eye is corrected automatically with the fluidic lenses described in U.S. Pat. No. 8,409,278, the disclosure of which is hereby incorporated by reference as if set forth in its entirety herein. Alternatively, the images may be projected onto a screen in front of the eyes (as shown in FIG. 4B). In FIGS. 4D and 4E, the transitional lenses 2020 have virtually no shading for minimal light conditions, the transitional lenses 2022 have some shading for moderate light conditions, and the transitional lenses 2024 have heavy shading for strong light conditions. The transitional lens 2026 has a different shape than the transitional lenses 2020, 2022, 2024. The transitional lenses 2020, 2022, 2024, 2026 have no pigment in their pin hole central apertures 2028, and may be in form of solid or fluidic lenses.

In one embodiment, the virtual reality (VR) lens is made to function like a pin hole (e.g., as shown in FIG. 4D). In this embodiment, the lenses 2020, 2022, 2024, 2026 may have a central hole 2028 and the peripheral part of the lens is made with transitional lenses that respond to light and create a virtual hole in the center where all the light rays entering the eye are in focus eliminating the need for any refractive modification of the VR lens.

In one embodiment, the VR lens is made to function like a pin hole by creating at least two concentric peripheral zones, and an inner central zone defining a visual axis. The polymeric material in the peripheral zones contains at least one light-activated chromophore that darkens when activated by light, the chromophore is dispersed in or on the outer surface of the lens polymeric material, distributed in substantially concentric circles outward from the central area, and uniformly increasing in concentration from the central area to the outer periphery; the central zone lacking the chromophore, or containing a chromophore that does not absorb visible light, or containing a chromophore at a minimal to zero concentration (see FIGS. 4D and 4E).

In one embodiment, the pinhole lens is made of two composite polymeric lenses, which includes a larger outer lens part with chromophore and a smaller central lens part of 1-4 mm that does not have the chromophore. The smaller lens is able to fit inside the larger lens. Alternatively, the inner part of the lens is a simply a dark tube, and functions as a pinhole that fits inside the outer part of the lens.

In one embodiment, the center of the lens is just a hole of 1-4 mm in diameter and has no lens whereas the peripheral portion has chromophores that change the color and the transmission of light depending on the density of the chromophores from light to very dark leaving the central area free through which the light passes.

In one embodiment, the pinhole arrangement of the VR lens eliminates the optical aberrations of the eye and also eliminates the need for accommodation or convergence.

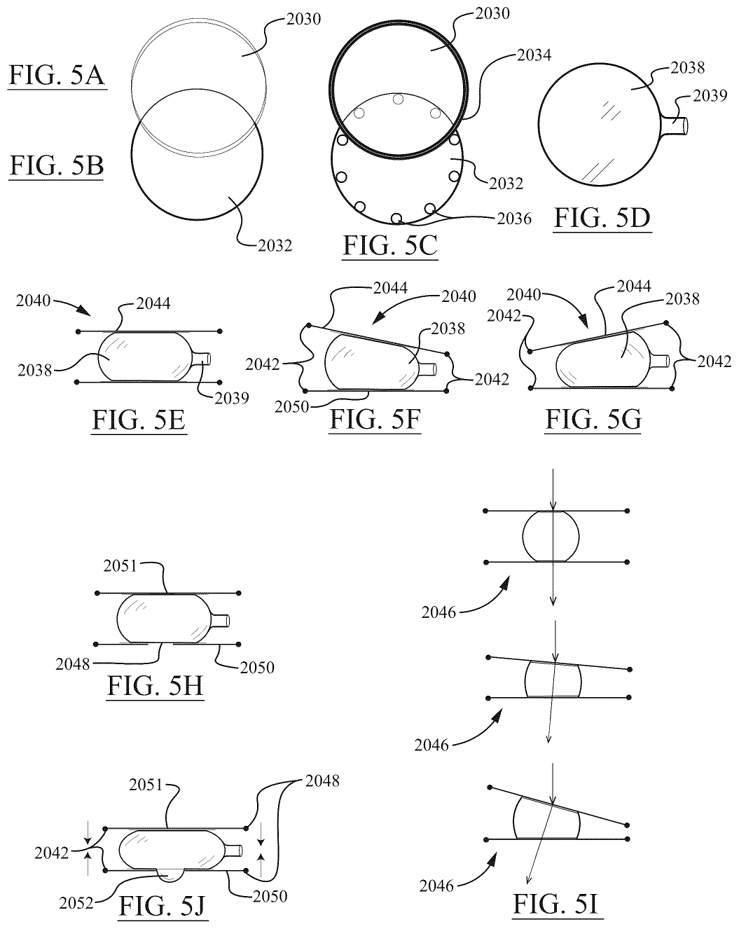

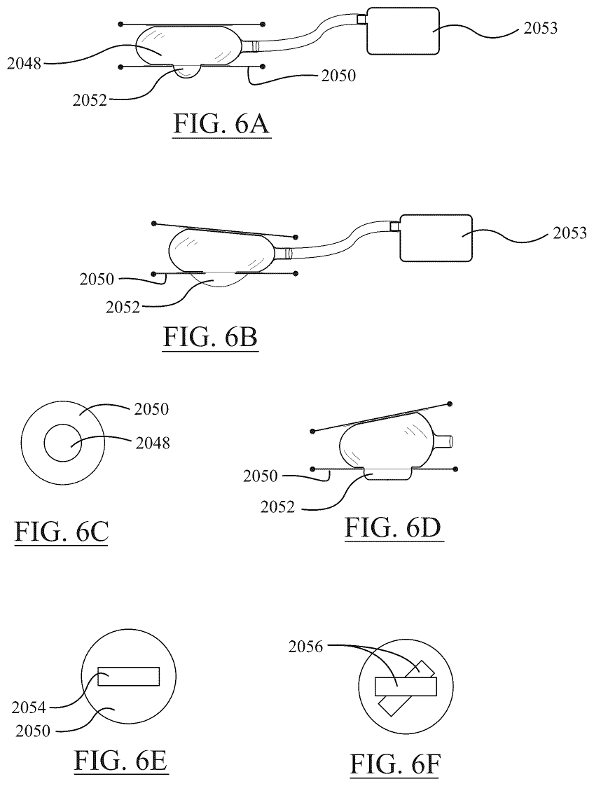

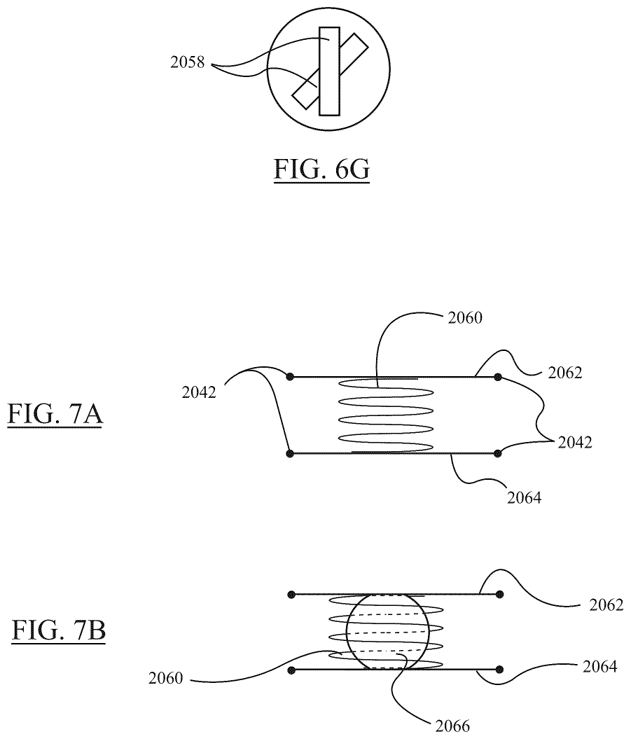

In one embodiment, if the need for convergence of one or another eye exists, one can use a fluidic prism in front of one or both eyes to correct for the pre-existing deviation, such as micro-strabismus <10 prism diopter (PD) or frank strabismus where the prism is made from a clear glass or polycarbonate, acrylic, stick-on Fresnel lens, etc. transparent to the visible and infrared light. In general, FIGS. 5A-5J illustrate the construction of a tunable or fluid prism. FIGS. 5A and 5B show two circular transparent plates 2030, 2032 of any size. FIG. 5C shows that the upper plate 2030 may have a magnetic material 2034 (e.g., iron) at its edges and the lower plate 2032 may have a series of electromagnets 2036 that can be activated independently or collectively via a data processor with appropriate software executed thereby. The balloon or the flexible transparent ball depicted in FIG. 5D is a transparent balloon 2038 made of silicone or another transparent clastic polymer which can be filled with a fluid (e.g., water or other transparent liquid) via a tube 2039 that can be connected to a pump, or is a transparent ball made of a transparent polymer. FIGS. 5E, 5F and 5G illustrate side views of a tunable prism 2040 showing unactivated or activated electromagnets 2042 electrically tilting the first or superior plate 2044 to one or the other side, thus creating a prismatic effect for the light passing through it. FIG. 5I illustrates side views of a similar tunable prism 2046 where the tunable prism is not activated in the first side view, the tunable prism 2046 is minimally activated in the second side view, and the tunable prism 2046 is further activated in the third side view. In the case of the flexible ball, all electrodes of the second plate are activated to collectively push the transparent polymer through a circular or rectangular hole, thus creating a spherical or astigmatic lens. FIGS. 5H and 5J show an alternative system. In FIGS. 5H and 5J, a central hole 2048 is cut in the second plate (i.e., the bottom plate 2050) through which the balloon 2052 protrudes outside the plate (as depicted in FIG. 5J) when the pressure inside the balloon 2052 is increased by the pump or by activating all the electromagnets 2042 simultaneously to compress the plates 2050, 2051 against each other. FIGS. 6A, 6B, and 6C show a circular opening 2048 in the back plate 2050 through which the balloon 2052 can protrude, thereby creating a tunable prismatic lens depending on the pressure applied to the balloon 2052 by a pump 2053. Similarly, FIGS. 6D and 6E show a rectangular opening 2054 in the back plate 2050 through which the balloon 2052 can protrude, thereby creating a tunable prismatic lens depending on the pressure applied to the balloon 2052. FIGS. 6F and 6G show the position of two superimposed tunable cylinders and tunable prisms 2056, 2058 located 45 degrees from each other that can be activated by a data processor with appropriate software, thereby creating a universal tunable astigmatic and tunable prism. In general, the prisms are solid triangular structures refracting the incoming light depending on the apex of the prism and its acuteness determines the prism's power (A), thus creating the displacement of the light rays (e.g., a displacement of one centimeter for a distance of one meter), and the light always deviates toward the base of the prism. The prisms are used to treat limited squints or strabismus that cause deviation of the eye caused by imbalance between the horizontal muscles or vertical eye muscles, or oblique muscles puling the eye toward one or the other direction. In general, coordination of the muscles between the two eyes is needed for both eyes to see an object simultaneously and prevent double vision. The simplest cooperation can be disturbed when objects are close to the eye requiring each eye to converge from their normal parallel position. The convergence is also associated simultaneously with an increase in the thickness of both crystalline lenses of the eye, the so called accommodation to focus at a near object (e.g. reading that is about 1-3 diopters). The function of convergence if not coordinated with the accommodation produces a commonly seen problem called strabismus or squint in children, such as in the esotropia, in which one eye more or less permanently turns in and with time loses its function (amblyopia) in order to prevent double vision. The external prisms are used by the ophthalmologist to measure the correct strabismus. Thus far, all prisms have static power (e.g., one to ten to twenty or more prism power). There is no known tunable prism.

In one embodiment, the fluidic or tunable prism is made of a flexible, transparent balloon 2038 located between two transparent plates made of glass, polycarbonate, etc. (e.g., see FIGS. 5E-5J). The function of the prismatic lens is always controlled electrically whereas the balloon 2038 displacement inside the hole 2048 in the second plate 2050 is done by a pump and the flexible ball is controlled only electronically. The balloon 2038 has an access to a tube 2039 through which the balloon 2038 can be filled with a fluid (e.g., air) or any other transparent liquid to be shaped like a basketball (i.e., round) or oval (i.e., like an American football), and the connection can be separated if needed without creating a leak when filled up with either gas, water, with or without electrolytes, silicone, or laser fluid having a specific index of refraction or the index of refraction is more than one or equal to one as the air, etc. or the balloon 2038 can be made from a soft transparent polymer, such as silicone or hydrogel, with a desired index of refraction, etc. The balloon 2038 is placed between two transparent plates. The first plate 2044 is moveable, and can be tilted in any direction, but the second plate 2050 is generally fixed to provide stability to the system, the plates 2044, 2050 are made of glass or any other material (e.g., acrylic) that is in contact with the surface of the balloon 2038, or the ball with the inner surfaces of both plates 2044, 2050 having some transparent adhesive. When the first plate 2044 is pressed toward the second fixed plate 2050, the balloon surfaces in contact with the plates 2044, 2050 flatten from two sides and the adhesive material fixes the two plates 2044, 2050 to the flattened central surface of the balloon 2038 creating initially two parallel plates with a central flexible balloon or ball in between (e.g., refer to the side views in FIGS. 5E and 5H), while the balloon 2038 or the ball edges can freely expand outward laterally between the two plates (e.g., see FIGS. 5F and 5G). In general, the position of the second plate 2050 is made stable by connecting it to any structure located nearby, such as a handheld holder or a part of the VR goggles, etc. The first plate 2044 is only connected via adhesives to the balloon 2038, otherwise the edges are free to move up or down or tilt due to the flexibility of its attachment to the balloon 2038 or the elastic ball. The free edges of the superior plate 2044 may have a magnetic element (see FIG. 5C), such as iron or iron oxide, etc., that can be tilted toward the edges of the second plate 2050 that has 4-12 or more electromagnets that can be turned on or off to generate a magnetic field or force that can be also controlled by the electrical current running in their coils. When the electromagnets in the second plate 2050 are activated, they attract the magnetic material of the first plate 2044 closest to it, and thus tilt the first plate 2044 toward that direction. The two plates 2044, 2050 can, in general, be positioned in any desired way (e.g., they could be one before the other, side by side, or one above the other, up and down position, etc.). However, the first plate 2044 can move freely since its attachment is with a flexible balloon 2038.

In one embodiment, the first plate 2044 can be a diffractive lens, a Fresnel plate with a desired prismatic effect or a holographic optical element rendering other functions to the plate.

In one embodiment, one can replace the balloon 2038 with a spring 2060 (e.g., see FIG. 7A, showing a single spring 2060 positioned between two transparent plates 2062, 2064 with their electromagnets 2042 that can tilt the first plate 2062 toward the second plate 2064 at any direction). The spring 2060 can be multiple small spring coils located around a central circular area through which the light passes from the first plate 2062 to the second plate 2064. FIG. 7B depicts an alternative combination of a spring coil 2060 and balloon or ball 2066, in which the balloon or ball 2066 is located inside of the central spring 2060 acting as a combination of a tunable prism and tunable lens that provides similar flexibility in motion to the first plate 2062. The advantage of a balloon or ball 2066 is that it can have an index of refraction chosen to be similar to air, or a higher index of refraction to create the bending of the light that passes first through the air and the glass plate 2062, then the balloon 2066 and the second plate 2064. The most important part of this invention is that the position of the first plate 2062 relative to the second plate 2064 influences how light travels through the two plates 2062, 2064, and when the first plate 2062 is tilted, it acts like a prism for refracting the light. When the two plates 2062, 2064 are parallel, the light enters the first plate 2062 in a perpendicular manner or normal manner, i.e., the light does not change its direction. However, if the superior plate 2062 is tilted in one or the other direction in relation to the second plate 2064, it creates a condition as seen with a prism. In this situation, the first plate 2062 acts as a side of the prism diverting the light toward the base of the prism (not the apex). As a result, one can control the degree and the location of the tilt of the first plate 2062 electromagnetically, as having a universal tunable or fluidic prism that now can be used or activated precisely electronically by an ophthalmologist by creating a precise electrical field at any desired location or direction that one would like, so as to act like a prism with the desired prismatic power that can be precisely controlled via a software that regulates individual electromagnets located at the perimeter of the second plate 2064 (e.g., activating the electromagnets of the right part of the second plate 2064 tilts the superior plate 2062 precisely toward the second plate 2064 by a desired certain degree to the right depending on the magnetic force generated in that area (e.g., see the side views in FIGS. 5F and 5G) from one prism degree (PD) or tilt to 30 degree PD or more tilt, the system thus converts two simple transparent glass panes into a prism with precise control toward any direction creating a universal tunable prism.

In one embodiment, a simple spring coil can be controlled as the tunable prism, and the simple spring coil is simple to create.

In one embodiment, the liquid or tunable prism or is combined with a spring coil that provides stability to the system by returning the plate to the parallel position when the electromagnet is not activated. In this embodiment, the central balloon or ball 2066 is positioned inside the spring coil 2060. The spring coil 2060 can be made from a plastic material or metallic material, but a plastic spring coil 2060 can work as well as the metallic one (see FIG. 7B).

In another embodiment, a spring 2060 of any diameter and coil number, which can be made of a plastic or any other material (e.g., a combination of metals), can be placed and glued around the center of the two transparent plates 2062, 2064 having otherwise similar electromagnets and magnetic materials as described. In this embodiment, the plates 2062, 2064 are in a parallel position to each other when the magnets 2042 are not activated (see FIG. 7A).

In one embodiment, with reference to FIGS. 5H and 5J, a tunable prism has a flexible balloon or ball 2052, and a central circle 2048 is cut out in the transparent second plate 2050 so that the balloon or ball 2052 can bulge out through the opening 2048, thus creating a plus lens, or in this case, a combination of a tunable refractive lens and a tunable prism simultaneously for correcting the eyes prismatic deviation and the required power of the lens needed (e.g., during convergence and accommodation). In addition, by injecting or removing the fluid from the balloon 2052 via a controlled pump, one automatically can increase or decrease the power of this tunable fluidic lens. In the case where the tunable prism has a ball, the magnets are all equally activated to compress the front plate against the second plate, and to cause the bulging of a part of the ball through the central opening in the second plate. This unit can be used as described in U.S. Pat. No. 8,409,278, which is hereby incorporated by reference as if set forth in its entirety herein, along with a Shack-Hartmann system for automatic control of the refractive power needed using a data processor with appropriate software loaded thereon. In general, the degree of accommodation needed for near work is between plus 1-5 dioptric power.

In one embodiment, one can collectively activate all electromagnets to compress the two plates toward each other, thereby enhancing the effect of the power of the lens/prism combination system (e.g., see FIG. 6B) so as to enhance the spherical or cylindrical power.

In one embodiment, where the opening in the lower plate is made oval or rectangular, one can create a combined tunable cylindrical lens and tunable prismatic plate, while the power of the lens is adjusted as needed using a pump system as described in U.S. Pat. No. 8,409,278 in combination with a Shack-Hartmann sensor and the power of the ball is controlled electrically by activating the electromagnets.

In one embodiment, two combinations of prismatic and cylindrical lens can be positioned at 45 degree angle to each other (e.g., refer to U.S. Pat. No. 8,409,278), thus correcting the amount of plus lens and the cylinder is needed for perfect correction of one or the other eye, or both eyes.

In one embodiment, the lenses can be combined with a Shack-Hartmann system as described in U.S. Pat. No. 8,409,278 with a pump connected to the balloon to correct tunable spherical and cylindrical and prismatic changes in one eye simultaneously. In this embodiment, a data processor with the appropriate software loaded thereon initially corrects the prismatic changes of the system, and then subsequently the spherical or cylindrical aberration of the eye.

In one embodiment, an additional spherical correction can be done where a fluidic lens as a minus lens is used independently (see U.S. Pat. No. 8,409,278) from the above system for myopic correction of the eye, but controlled by the same Shack-Hartmann pump and a software.

In one embodiment, one should eliminate the factors that predisposes or contributes to a person having side effects of using the virtual reality or augmented reality systems by performing a complete examination of visual functions, disparity of optical aberrations of one or both eyes, history of strabismus or micro-strabismus, history of nystagmus, ocular surgery, cornea, crystalline lens, retinal diseases, or genetic or acquired diseases affecting the eyes by addressing each independently and correcting for them, if possible.

In one embodiment, the patient might have strabismus, that is, one eye deviates from the other eye more than one prism diopter (e.g., one centimeter for a distance of 100 cm) when looking at an object, thus causing disparity of the images that is projected over the central retina (fovea) creating double vision. The misalignment is esotropia or inward convergent and exotropia, hypertropia, hypotropia, incyclotorsion or excyclotorsion, etc. The problem can be stimulated during accommodation, often seen in children around the age of 2 to 3 when looking at a near object or without accommodation, and their magnitude can be measured by a handheld Fresnel prism. Mechanical esotropia is caused by scar tissue or myopathy, etc. and requires surgical correction of the underlying disease process.

In one embodiment, the disparity of the images can be addressed by two independent mechanisms, which first include correcting the convergence deficiencies or pre-existing microtropia or macrotropia of the eye which stresses the eyes during the convergence. This problem should be addressed by a prior examination using an innovative auto-phoropter system to measure the aberration of the refractive power of the eye, and automatically correct the refractive power. In one embodiment, the phoropter is combined with an adjustable or tunable prism to correct refractive error and the eye deviation. These issues can be treated prior to the use of the VR or AR system, but some other issues, such as amblyopia, that have existed from childhood as a result of not using both eyes together, etc. may or may not be corrected depending at what age they have been discovered. The treatment of this condition is done by covering the good eye for a period of time to force the person to use the weaker eye until the visual acuity becomes normal or close to normal.

In one embodiment, the adjustable prism is prescribed, but slowly reduced when the eye muscle becomes stronger to eliminate potentially the need for a prism.

In one embodiment, the convergence deficiencies may be corrected by surgery of the eye muscles or by positioning appropriate prisms in front of the eyes to bring the images of the two eyes together. This can be done by presenting to the eyes two independent images having red or green letters or a number, or using a Maddox rod presenting the eyes with a colored astigmatic lens that separate the images of both eyes and shows how the two eyes cooperate to unify the image or how the two separate images seen by each eye cooperate and can then be corrected by specific prisms or a tunable prism directing the image toward the eye or unifying them.

In one embodiment, dyslexia might contribute to separation of images seen by each eye and can be diagnosed by having the patient read a reading chart so that the optometrist or ophthalmologist may diagnose the condition.

In one embodiment, one evaluates the existence of nystagmus diagnosed by presence of a visible oscillatory motion of the eye, which can be barely visible, but can be examined using appropriate testing to recognize it prior to the use of the VR or AR goggles, or can be treated by limiting the oscillation by positioning an appropriate prism on each of the eyeglasses that might help the nystagmus to a certain extent, or the electrical pulses to the ocular muscles is dampened by administration of a topical medication, or injecting Botox inside the muscles.

In one embodiment, the nystagmus can be brought under control by reducing external light using transitional lenses that leave the central 2-4 mm area free of pigment and darkening mostly the astray light coming from the sides that cause glare, headache, and the sensation of vomiting and aggravate the effect of the symptoms of seasickness.

In one embodiment, these aforementioned tests will eliminate patients having one or more ocular problems, and/or they will help manage their problems prior to use of the VR goggles.

In one embodiment, in a VR headset, one can automatically correct the prismatic changes by rotating the direction of the light (image) coming to each eye independently until they correspond to form a single stereoscopic image or incorporate an adjustable prism combined with the lens to divert the light appropriately to each eye.

In one embodiment, one can manipulate the degree of stereovision by creating a lesser stereoscopic effect to no stereovision in order to eliminate the side effect of motion sickness by creating more or less stereovision gradually to enable the user of the VR or AR headset to get used to the increased stereoscopic view by exercising and using the concept.

In one embodiment, since the side effect of the visualization using VR is dependent on the degree of stereoscopic vision (i.e., more or less stereoscopic), the angulation of the light entering the pupil can be adjusted gradually until the person feels comfortable looking through the glasses of the VR headset.

In one embodiment, lenses are provided, which can act as a pinhole, to provide the best focusing condition for the eye to see since the light rays are positioned directly on the fovea of the retina without any diffraction of them from the side of the optical element of the eye, cornea, and the lens. It also eliminates the need for accommodation that induces simultaneous convergence that exhausts the ocular muscles.

In one embodiment, the pinholes lenses are specifically designed to create a pinhole in presence of and degree of light.

In one embodiment, the nystagmus can be recognized using optokinetic nystagmus, rotating cylinder with black and white stripes creating symptoms of seasickness.

In one embodiment, the dizziness, etc. can be diagnosed by monitory head and eye movement continuously with a device called Continuous Ambulatory Vestibular Assessment (CAVA) device.

In one embodiment, since the visual confusion and position of the body can complement each other worsening the symptoms, various eye tracking following the eye movement and accelerometers can track the body or head motion and sensors checking the physiological changes of the body can be coordinated by a processor to reduce the fast position changes of the VR images so as to reduce the symptoms.

In one embodiment, this is achieved by seeing two images simultaneously in the path of each eye, one image provides a stable frame, such as two or more vertical bars with 1-2 horizontal bars in relationship to the observer's body so that the user of VR can focus on or practically ignore it, while observing the VR image independently and providing an anchor for the viewer that creates a sensation that he or she is looking through a transparent motionless frame at the VR, through the rectangular window provided for the eye. This sensation is not different than the fear of height. These persons usually freeze if they are on a high building or platform that is not providing a feeling of separation from the outside view of the “world” from the person's position such as it would be seeing through a transparent glass, fixed to a structure providing a security of separation from the outside world lying below him or hers, and in front of the person which is seen stereoscopically.

In one embodiment, one can create a barrier that works like a window shutter with a transparent glass that separates the outside world which is visible through the transparent or semi-transparent glass with or without the vertical or horizontal bars. In one embodiment, the user's problem with the virtual reality is treated by projecting the 3-D images on a heads-up screen, then projecting the images on a computer screen in front of the eyes, thus providing the sensation of being outside the scene rather than inside the scene, and eliminating the neuronal side effects or vertigo or seasickness where the patient is, or imagines to be inside the scene.

In one embodiment, by creating either a second separate transparent or semitransparent goggle cover or another two dimensional virtual glass located in an area in the front of the VR image that appears stable having some stable images on it whereas the VR is seen in 3-D beyond it, so that the person can focus on the first “transparent glass barrier” before seeing the 3-D VR, to get relief from the stereoscopic images that cause the visual confusion and mental discomfort. A double transparent platform with stable vertical and horizontal marking edges on the inside glass creating a static frame of reference between the two different, but connected spaces in the visual field. Thus separating the two spaces from each other, like entering one room first and then entering the second room (i.e., the virtual room).

In one embodiment, by creating either a second separate transparent or semitransparent goggle cover or another two dimensional virtual glass located in an area in the front of the VR image that appears stable having some stable images on it whereas the VR is seen in 3-D or as a hologram beyond it, so that the person can focus on the first “transparent glass barrier” before seeing the 3-D VR, to get relief from the stereoscopic images that cause the visual confusion and mental discomfort.

In one embodiment, the outside glass space has the VR images and the inside glass has only the limiting bars giving the impression of a separate space from the VR that separates the VR world from the real world (or space). The bars can be virtual so that their position or location can be changed depending on accelerometers, other sensors, or an eye tracking system located on the VR headset indicating the direction of the visual/head movement. These signals are transmitted to the frame or bars of the first space, changing the position of the virtual frame depending on the inclination or the head tilt, moving the image against the force of the gravity, to maintain a relative vertical and horizontal stability to the area in front of the VR space.

In one embodiment, the system described can additionally have stable frames projected in the path of vision eliminating the fear (of VR) similar to that of being on an elevated area, but being inside another transparent space which is separated from the stereoscopic VR images or hologram providing comfort of security for the viewer.

In one embodiment, one can also make the “supporting” image moveable from one direction to the other so that the image remains constant either in the vertical or horizontal level. This is achieved by having one or multiple accelerometers and sensors positioned around the goggles that indicate the position of tilt of or forward/backward motion, connected to a processor that adjusts automatically the position of the supporting image in a horizontal and vertical position, alleviating the visual sensation of rotation and tilt that comes with looking through the VR systems.

In one embodiment, depending on the tracking system or the sensors sensing tilt, etc., one can stimulate the neck muscles by electric pulses applied to the muscles in one or the other direction to loosen up the muscle spasm, loosening the fixed rigidity created during the motion sickness or blocking the vagus nerve stimulation by electrical pulses to depolarize the nerve or to depolarize the oculomotor nerve controlling the eye movement and ocular muscles that otherwise would result in stretching or traction of extra-ocular muscle.

In one embodiment, if the sensors, accelerometers, or other body sensors or wrist sensors indicate physiological changes of the patient, a processor can control the VR frequencies of pulses instead of providing 60-100 or more light pulses of the image per second, the presentation of the image can be reduce to 4-8 images per second by a processor automatically to relieve the person's symptoms until the side effects are subsided. This provides an automatic relief for the observer from the motion sickness by reducing the stereovision from 3-D to 2D images.

In the previous patents, it has been described how fluidic lenses seen through phoropter can produce an objective refraction when it is combined with a Shack-Hartmann sensor and the software to control the amount of the fluid in the lenses producing plus or minus lenses. For example, refer to U.S. Pat. Nos. 7,993,399, 8,409,278, 9,164,206, and 9,681,800, the disclosure of each of which is hereby incorporated by reference as if set forth in their entirety herein. When placed in front of an eye, human or animal, the phoropter can produce an objective refraction without the need for a subjective verbal or non-verbal communication to the doctors or technicians and the patients. The phoropter makes objective non-verbal the examination possible.

In one embodiment, the refractive errors of each eye of the newborns are examined separately using an objective fluidic hand held phoropter.

In one embodiment, similarly the refractive error of each eye of animals, such as dogs, cats, horses, etc., can be measured using a fluidic hand held objective phoropter.

A more permanent correction of the refractive errors requires a set of glasses that can either be easily adjusted or a hybrid system that at a minimum, corrects refractive errors for far and near for each eye. At present, these lenses are not used for humans or animals.

Similarly, there has not been a need to check the refractive error of an animal for the lack of the communications. However, there is no objective studies performed on refractive errors of the animals. Probably, the animals with severe refractive errors do not live the full life because of their visual deficiencies, and poor vision. This issue becomes more important for the domesticated pets, such as cats, dogs, horses, particularly racing horses, and other animals. Poor vision can also cause these animals to trip themselves, fall, or break their legs, etc. as it is the case with older humans.

Although, the fluidic phoropter can solve this problem, no attempt has been made to correct the refractive errors in animals and babies.

In one embodiment, the fluidic lenses can eliminate the barrier of expenses and provide a multifocal fluidic, or hybrid fluidic refractive glasses for the babies and the animals that can be used with the potential of being readjusted within six months as the eyes grow or as the need dictates. These lenses can be used also in virtual reality or augmented reality goggles, thereby eliminating the eye strains or headache seen in these users.

In a pending nonprovisional application, namely U.S. Nonprovisional patent application Ser. No. 17/134,393, the present inventor has described the application of modified refractive surgery technique for the human and the animals that are reversible without the need for removing the tissue from the eye as is done presently.

Refractive error of the eye constitutes one of most common visual problems affecting billions of the population worldwide. These refractive errors deprive the affected person not only from proper development of the vision (e.g., in children if not corrected but also contribute to loss of sight, the so-call amblyopia or lazy eye in which the ability to see from one eye can be permanently is lost if not corrected at young age).

Often the lack of access to an ophthalmologist or optometrist contributes to the loss of sight. However, often the lack of financial ability also contributes to loss of education and productivity of a person throughout his or her life.

In general, the majorities of the refractive errors are myopia (nearsightedness) where the eye is too long or the corneal curvature is too steep preventing the light to be focused on the retina but falls in front of it. This condition requires a minus or a concave lens for its correction to move the focal point of light backwards towards the retina. The hyperopia is a condition where the refractive power of the eye (crystalline lens and the cornea) is not enough and causes the light rays to be focused behind the retina. This condition can be corrected by the use of a convex or a plus lens or glasses to bring the focal point of the light rays forwards towards the retina. Presbyopia is an aging problem in which the normal crystalline lens loses its elasticity to focus on the near objects, such as reading a newspaper where a plus lens can treat the condition.

In general, a combination of a plus lens added to another corrected lens for the far can provide bifocality to the glasses that can be used when the patient looks through the upper part of the glasses seeing far objects and during the reading the person looks down through the second lens, usually in the lower part of the bifocal glasses and can read a newspaper. It is also possible to create triple focal lenses that provide sharp vision for three distinct distances from the eye (e.g., far correction using the upper part of glasses, near within a comfortable distance, e.g., for an orchestra conductor to read the music chart, i.e., about 3 feet, and near section for reading in a distance of 33 centimeters or a about a foot). This requires each glass section to be corrected each individually. In general, a three step procedure is performed to achieve a multifocal lens. The far distance is initially corrected by measuring the refractive error for spherical correction for distance by the fluidic lens by one adding various plus lenses that can provide a focal point to a distance of 10-30 centimeters, 30-60 centimeters, 1-3 meters, and 3-5 meters, depending on the need of a person's eyes (e.g., babies require to seeing very near objects and intermediate distances, while for animal, an intermediate distance and far are initially more desirable). However, either adding a plus lens or a diffractive lens can cover these intermediate distances, and adult humans would like to see far and near and all eyes can be blinded by excessive light or side light when working or playing under the sun or bright light, and are in need of blocking excessive light, such as by using transition lenses that have light absorbing pigment in the lens. The latter or a combination is very desirable for an albino patient who does not have much dark pigment to block the sun light. The three step procedure for the optical correction encompasses: (1) objective measurement of the refractive power using an automated hand held objective refractometer and phoropter, (2) assembling the system as described in this application, and (3) checking the accuracy of the desired dioptric power of the hybrid lens using the standard lensometer, and the refractive error of the desired power is corrected while the hybrid lens is under the lensometer to achieve the prescription power measured with a system including an objective phoropter, fluidic lenses, and a Shack-Hartmann sensor assembly. The process of changing the refractive power of the hybrid lens to achieve the refractive power of the fluidic lens for far is done by activating the step motor head or a hydraulic pump that are connected to the flexible membrane of the initial chamber that is, in turn, connected to the fluidic lens chamber via a conduit to the fluidic lens's chamber having a flexible membrane acting as a lens. The pushing or pulling of the step motor head is activated either electronically or mechanically that either pushes or pulls, forwards or backwards, the flexible wall of the initial small chamber. The amount of push/pull of the step motor can be electronically or mechanically controlled to change the dioptric power of the fluidic lens membrane as the refractive error is measured simultaneously under a lensometer and finally the interpupillary distance is measured/adjusted and the glasses are positioned with their frames and holder that can be an elastic band with a hook-and-loop fastener (e.g., Velcro®) behind the car or behind the head and kept stable.

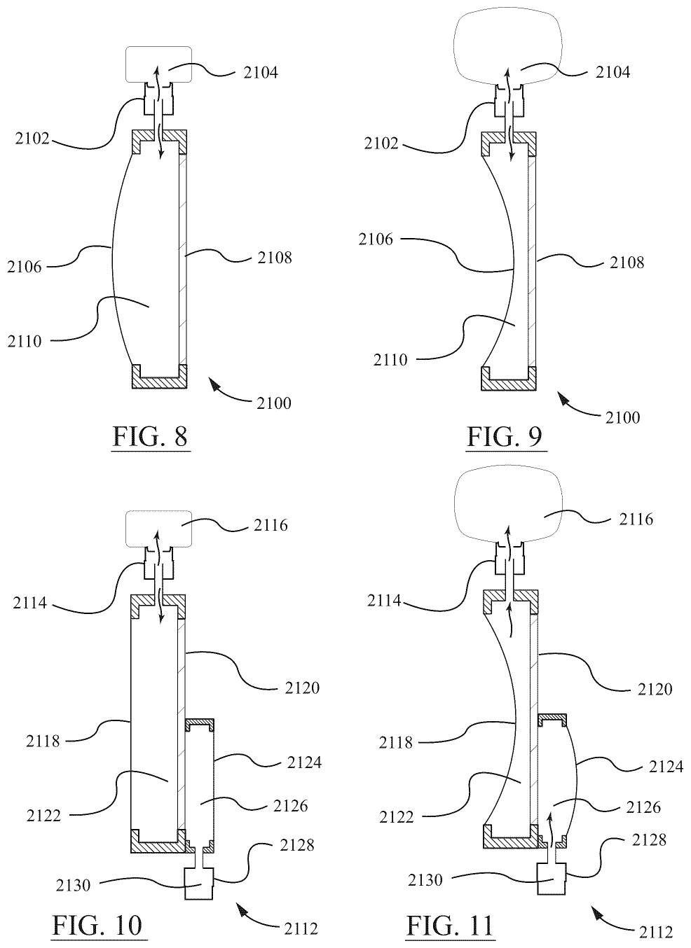

In one embodiment, binocular glasses are made out of two fluidic lenses or a combination of a two separate fluidic lens that build two different chambers, but share a solid transparent barrier in between them in which the amount of fluid in each chamber is increased or decreased to provide the upper part for the far vision and is fixed at that position and the lower “back” chamber provides only a plus or addition of +1-3.00 D. and is fixed to serve for the reading. However, the fluidic lenses can also be adjusted if the patient's eye changes its power as the eye grows (e.g., in children) and requires a different kind of power and can be adjusted by the patient (refer to FIGS. 8-11).

An illustrative embodiment of a corrective fluidic lens 2100 with a flexible membrane 2106 is depicted in FIGS. 8 and 9. As shown in these figures, the flexible membrane 2106 is supported in an outer housing with a solid flat glass plate 2108 forming the back of the housing. The flexible membrane 2106 and the outer housing together define an internal fluid chamber 2110 for receiving a fluid therein (e.g., a laser fluid with a high index of refraction). A fluid reservoir 2104 is fluidly coupled to the fluid chamber 2110 of the fluidic lens 2100 so that the fluid may be injected into, or withdrawn from the fluid chamber 2110 by means of a fluid pump (e.g., the fluid pump 2102 in FIGS. 8 and 9). In FIG. 8, the flexible membrane 2106 is disposed in a convex configuration for correction of farsightedness of an eye (hyperopia). In FIG. 9, the flexible membrane 2106 is disposed in a concave configuration for correction of nearsightedness of an eye (i.e., myopia).

An illustrative embodiment of a presbyopia bifocal fluidic lens 2112 with two fluidic chambers 2122, 2126 for correcting both hyperopia and myopia is depicted in FIGS. 10 and 11. As shown in these figures, the front flexible membrane 2118 is supported in an outer housing with a solid flat glass plate 2120 forming the back of the housing. The front flexible membrane 2118 and the outer housing together define an internal fluid chamber 2122 for receiving a fluid therein. A first fluid reservoir 2116 is fluidly coupled to the fluid chamber 2122 of the fluidic lens 2112 so that the fluid may be injected into, or withdrawn from the fluid chamber 2122 by means of a fluid pump (e.g., the fluid pump 2114 in FIGS. 10 and 11). As shown in these figures, the rear, smaller flexible membrane 2124 is supported in an outer housing with the solid flat glass plate 2120 forming the back of the housing. The rear, smaller flexible membrane 2124 and the outer housing together define an internal fluid chamber 2126 for receiving a fluid therein. A second fluid reservoir 2130 is fluidly coupled to the fluid chamber 2126 of the fluidic lens 2112 so that the fluid may be injected into, or withdrawn from the fluid chamber 2126 by means of a fluid pump (e.g., the fluid pump 2128 in FIGS. 10 and 11). In FIG. 10, the flexible membranes 2118, 2124 of the presbyopic bifocal fluidic lens 2112 are both disposed in flat, relaxed states. In FIG. 11, the front flexible membrane 2118 of the presbyopic bifocal fluidic lens 2112 is disposed in a concave configuration for correction of myopia, and the rear flexible membrane 2124 is disposed in a convex configuration for correction of hyperopia.

In one embodiment, a fluidic lens serving the far vision is located in front of a plane of clear glass or acrylic plate and the second fluidic chamber forms a part of a chamber located on the lower part of the back surface of the first chamber and slightly inferior and the back part of the front chamber sharing, for example, the clear acrylic plate, methacrylates (e.g. (poly) methacrylates), (hydroxyethyl) methacrylate (HEMA), silicone, glass, etc. Polymers are known in the art and may be organic, inorganic, or organic and inorganic (see FIGS. 10 and 11).

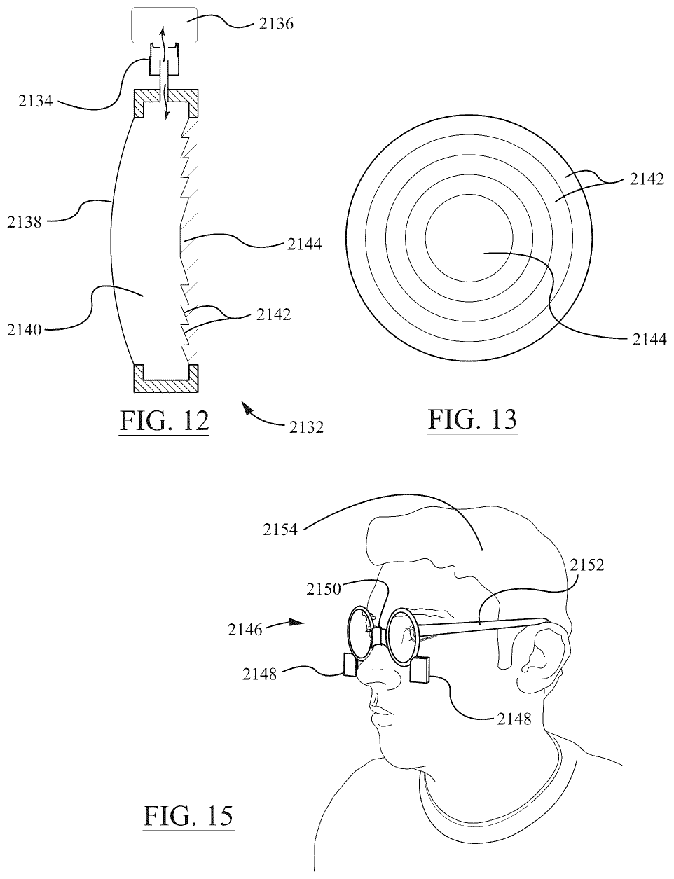

In another alternative embodiment, the fluidic lens comprises a front chamber with a flexible membrane that can act as both a positive and negative lens, while the back side of the chamber is a standard diffractive lens in which a standard Fresnel lens with multiple zones of prisms are created that provide the standard plus zones that provide many diffractive fixed plus zone focal points (refer to FIGS. 12 and 13). These zones can serve only for presbyopia correction or near vision, while the fluidic lens is precisely corrected for the far vision (refer to FIGS. 12 and 13). These zones are not made of liquid crystal in which changes in refractive power is limited to +2-3.00 D and is controlled electronically via a switch.

An illustrative embodiment of a diffractive fluidic lens 2132 having a front fluidic lens chamber 2140 with a flexible membrane 2138 and a rear Fresnel diffractive lens 2142 with multiple zones of prisms to provide many fixed diffractive plus zone focal points is depicted in FIGS. 12 and 13. As shown in FIG. 12, the flexible membrane 2138 is supported in an outer housing with a Fresnel diffractive lens 2142 forming the back of the housing. The Fresnel diffractive lens 2142 has a central region 2144 (see FIGS. 12 and 13). The flexible membrane 2138 and the outer housing together define an internal fluid chamber 2140 for receiving a fluid therein (e.g., a laser fluid with a high index of refraction). A fluid reservoir 2136 is fluidly coupled to the fluid chamber 2140 of the fluidic lens 2132 so that the fluid may be injected into, or withdrawn from the fluid chamber 2140 by means of a fluid pump (e.g., the fluid pump 2134 in FIG. 12). A top view of the rear Fresnel diffractive lens 2142 of the diffractive fluidic lens 2132 is shown in FIG. 13.

In one embodiment, a combination of a set of fluidic lens and a set of multifocal lens provide a new set of glasses for a person as young as a few months (or animals) or as old as 100 years or more.

In one embodiment, one can correct far vision for both eyes to create stereo-vision via a fluidic lens, while a diffractive lens provides for near and intermediate vision without the need to be activated electronically, etc. since these light rays are all in focus for different near distances.

In one embodiment, the fluidic lens can be replaced with a tunable lens, such as Optotune lenses, etc. that works electronically, but needs to be adjusted many times during the day by the patient to create multifocality. In contrast, the hybrid liquid lens and diffractive lens described above does not need to be adjusted each time for each given distance.

In one embodiment, the fluidic lens is corrected for the far for each patient depending on their refractive aberration and the diffractive lens provides automatically multifocal points of fixations for different distances from the eye for the objects located in the near field from 33 centimeters to 6 meters or more all the time. This modality specifically is useful for very young children or animals with limited accommodative power of their crystalline lens or in aphakia. Using these combinations, the objects located at different distances from the eye in the outside world are always in focus for the eye eliminating excessive accommodation that is needed, which is accompanied by excessive convergence needed for the near object.

In one embodiment, the diffractive Fresnel lens constitutes a flat surface with its Fresnel fixed zones that could be used for any person having 2 to 3 or more standard zones as needed.

In one embodiment, only the fluidic lens section is initially corrected for the far vision in each eye and does not require to be turned on or off electronically or adjusted daily, many times.

In one embodiment, the fluidic lens provides either a positive or negative lens for the eye while the diffractive lens provides zones of plus zones covering for a correction of near vision of 1 centimeter to 10 meters as desired for each eye independently depending on the zones of the Fresnel lens.

In one embodiment, there are only two Fresnel zones covering two distinct distances from the eye (e.g., 10 centimeters to 100 centimeters), while in another embodiment, the zone may cover a distance of 10 centimeters, 30 centimeters, or 500 centimeters or more depending on the patient's need, such as in young children or in adults.

In one embodiment, the combination of diffractive with fluidic section is specifically useful for AR (augmented reality) and VR (virtual reality) vision goggles eliminating the need for convergence and subsequent headache that are seen in a large number of the patients after use of these devices.

In one embodiment, means are provided for the fluidic lens chamber to be connected to a pump that can be activated mechanically or electronically to push the fluid inside the chamber or remove it to create a plus or minus lens from the surface of the flexible membrane covering the lens chamber providing lenses with +0.5 to >+20.00 Diopter power or −0.5 D to >−20.00 Diopter power.

In one embodiment, the fluidic lens can be used with an electrically tunable lens and a diffractive lens

In one embodiment, the glass plate behind the fluidic lens is made out of a transparent plate with diffractive zones.

In one embodiment of a hybrid lens, the tunable fluidic flexible membrane is changed to the desired optical power for the distance by injecting or withdrawing the fluid from its chamber and the astigmatic correction of the transparent acrylic plate, located at its back, is made to the desired axis and power using a femtosecond laser with pulses of 5 to 10 or more nano-Joules that changes the index of the refraction of the acrylic lens at the desired axis without damaging the lens.

In one embodiment, in the presence of microstrabismus (eye deviation) of the eye, the tunable fluidic lens is in the front part of the hybrid lens and is corrected for the far vision, whereas the lens transparent back plate can be made from a prism of 1-10 prism power that can be placed in any direction to correct microstrabismus that causes headache in patients suffering from microdeviation of one eye.

In one embodiment, the microstrabismus is in a horizontal position and the prismatic correction is made with the transparent back plate to correct horizontal eye deviation in order to facilitate stercovision.

In another embodiment, the microstrabismus is in a vertical position and the prismatic correction is made with the transparent back plate to correct vertical eye deviation in order to facilitate stercovision.

In one embodiment, the front fluidic lens is made to correct the refractive error for the distance while another fluidic lens bordering the back surface of the first (front) is made to be positioned in the lower half of the front lens to act as presbyopia correction (refer to FIGS. 10 and 11).

In one embodiment, the astigmatic correction is made for both eyes as needed with a femtosecond laser with low energy pulses of 5-10 or more nano-Joules at the desired axis to provide correction for astigmatic error of the eye to facilitate stercovision.

In one embodiment, a hybrid fluidic lens is in front and a transparent glass or acrylic plate is in the back (see FIG. 8), with a fluidic pump located on the upper part for injection of the fluid producing a convex or plus lens as needed.

In another embodiment, the fluid is removed by the pump system creating a concave or minus lens from the flexible membrane (see FIG. 9) with the desired dioptric power. The fluidic material is laser fluid, or mineral oil etc. with a high index of refraction.

In another embodiment, two chambers are used for the fluidic lenses, which are separated by a glass plate of acrylic transparent plate or another transparent molecule plate, such as polycarbonate etc. (see FIGS. 10 and 11). The front fluidic lens acts to correct the far distance vision by injecting the fluid in it to become a convex lens or plus lens of +0.1 D to +20.00 D, or removing the fluid to become a concave lens or minus lens of −0.1 to −1.00D to −20.00 D spherical lens, while the back lens is used for creating a presbyopia lens of +1.00 to +3.00 D power.

In one embodiment of a hybrid lens, the posterior plate can be modified with a femtosecond laser to change the index of the refraction of the lens to make it an astigmatic lens of +0.1 to +3.00 D or more astigmatic in a desired axis.

In one embodiment, the posterior plate is made from a diffractive plate (see FIGS. 12 and 13) to create a multifocal fluidic hybrid lens. In this configuration, the fluidic front lens corrects the distance vision, while the back diffractive plate corrects for intermediate distances of 10 centimeters to 600 centimeters, etc. The Fresnel zones are tightly packed to achieve multifocality for the glasses.

In one embodiment of the hybrid lens, the only correction made is with the front fluidic lens to achieve the distant vision with a power of +0.1.00-20.00 D power in convex or concave lenses of −0.1.00, −20.00 D, while the diffractive back plate creates multifocal lens in the back of the fluidic front lens to provide focal points for objects located from the eye to a distance of 6 to 8 meters or more, thus these lenses need to be corrected by injecting fluid only in the front chamber or removing the fluid from it, about once every six months or more for the distant vision correction only, without changing the lenses.

In one embodiment, chambers of fluidic lenses are separated from outside by a glass plate, polycarbonate or an acrylic transparent plate, etc. The transparent plate can be mixed with a light sensitive pigment or the light sensitive pigment, such as photochromic molecules, oxazines, and naphthopyrans, can be sprayed over its surface or inside the plate that render the plate to act like transitional lenses (i.e., FIGS. 14A-14D). That is, the plate becomes dark when the light shines over it to reduce the glare, whereas the color changes and the plate becomes transparent in the dark.

FIG. 14A depicts a top view of an illustrative back plate 2156 of a fluidic chamber of a fluidic lens, where the back plate 2156 is in the form of a transitional lens with a pigment that changes color based upon the amount of light absorbed. FIG. 14B depicts a top view of another illustrative back plate 2158 of a fluidic chamber of a fluidic lens, where the back plate 2158 is in the form of a transitional lens 2160 in which the pigment does not cover a small central area 2162 of the plate, thereby creating a pinhole configuration in the plate when the plate is exposed to light. FIG. 14C depicts a top view of yet another illustrative back plate 2164 of a fluidic chamber of a fluidic lens, where the back plate 2164 is in the form of a diffractive lens 2166 in which the central area 2168 of the plate is not diffractive, thereby creating a pinhole configuration in the plate 2164. FIG. 14D depicts a top view of still another illustrative back plate 2170 of a fluidic chamber of a fluidic lens, where the back plate 2170 is in the form of a diffractive transitional lens 2172 in which the pigment does not cover a small non-diffractive central area 2174 of the plate 2170, thereby creating a pinhole configuration in the plate when the plate is exposed to light.

In one embodiment, the back side glass plate of acrylic transparent plate is sprayed or mixed with a pigment that changes its color temporarily after exposure to the light, thus building a fluidic transitional lens.

In one embodiment, the pigment known in the art does not darken the lens permanently, rather it is a photochromic molecule (i.e., a molecule that is activated by light) and, upon activation, darkens the plate, such as photochromic molecules that are activated by ultraviolet (UV) light are oxazines and naphthopyrans, or by visible light is silver chloride or activated with light in the UV and visible spectrum, such as silver chloride multiple different chromophores, not limited to, those that absorb UV light, or those that absorb visible light, or those that polarize light, and combinations of these.

In one embodiment, the back plate of the fluidic chamber is made out of a glass plate or transparent acrylic plate, etc. in which the back plate contains pigment or pigment is sprayed on it to change the color by absorbing the light and turning dark (see FIG. 14A).

In another embodiment, the back plate of the fluidic chamber is made out of a glass plate or transparent acrylic, polycarbonate plate, etc. in which the back plate contains pigment or pigment is sprayed on it to change the color by absorbing the light and turning dark. However, the transitional lens or the pigment covers only most of the peripheral part of the plate and lightens up slowly within a distance from the center of the plate or stops within 2 to 7 mm in the central area of the plate creating a pinhole configuration in the plate when exposed to the light (scc FIG. 14B).

In one embodiment, the back plate of the fluidic chamber is made out of a diffractive lens with zones that are very closely packed with the focal point of the Fresnel zones are close to each other, and in one embodiment, the transitional diffractive lens pigmentation stops at a clear zone of a 2 to 7 mm circle (see FIGS. 14C and 14D), where the light remains mostly focused on the retina creating a variable pinhole effect depending on the intensity of the outside light.

In one embodiment of the hybrid lens, the diffractive back plate is mixed or its outer surface is sprayed with pigment selectively to build a pigmented doughnut shaped lens periphery where the central area of the lens builds a circle with a diameter of 2 to 7 mm or more free of the pigment, thus building a fluidic tunable lens in front and a transitional diffractive lens in the back (see FIG. 14D) with a central pinhole that stays clear all the time and having a pinhole effect on the vision (i.e., the light passing through this pinhole focuses always on the retina for objects located in the near or far in front of the eye).

In one embodiment, the above configuration permits the person to carry these lenses in the dark (i.e., at nighttime) and light (i.e., during the day) without being significantly blinded by the peripheral glare (e.g., from outside during the day, while being able to see at night).

In one embodiment, these lenses can be placed inside the VR or AR goggles to provide sharp images on the retina eliminating the glare and need for accommodation or convergence since the images are presented to each eye separately and are always in focus passing through the hybrid fluidic lenses with transitional ability.

In one embodiment, this new transitional hybrid lens with a central clear area forms a permanent pinhole that remains clear without the need to wait for the time to pass for the pigment to become clear after passing from outside through a relatively dark tunnel to be able to see and changeovers for the person to see outside a tunnel, since the pinhole area remains clear all the time, while the peripheral glare is eliminated by the transitional section of the diffractive lens (see FIG. 14D).

In one embodiment, the surface of the elastic fluidic lens membrane can be painted with the pigment to act similarly so as to act like a transitional lens if needed.

In one embodiment, the fluidic hybrid lens with its diffractive back surface can be used inside of any peripheral plastic holder with any “glass” configuration as circular, rectangular, oval, or elongated oval where the extreme sides can be bent backward to prevent side glare. In general, the central part of the glass can be circular with optics and its peripheral non-optical section can be clear or pigmented, etc. (see FIG. 15).

In one embodiment, the hybrid glasses are made with their side pump for babies, children, adults, or animals to a desired size that is comfortable for these subjects (see FIG. 15).

An illustrative embodiment of fluidic adjustable glasses 2146 disposed on a person 2154 is depicted in FIG. 15. As shown in FIG. 15, the fluidic adjustable glasses 2146 generally comprise a pair of fluidic lens (one for each eye of the person 2154), and a respective pump 2148 operatively coupled to each of the fluidic lens. The pumps 2148 are configured to insert an amount of fluid into the respective chambers of the fluidic lens, or remove an amount of the fluid from the chambers of the fluidic lens in order to change the shape of the fluidic lens in accordance with the amount of fluid therein. As shown in FIG. 15, the fluidic glasses 2146 include temples (arms) 2152 and a telescopic bridge 2150 for accommodating varying face widths.

In one embodiment, the frames can be made out of any polymers (e.g. acrylic, polycarbonate, etc.), or an elastic band made (e.g., from strips of silicone with an appropriate color) that can be locked behind the eye, or a mixture of acrylic and elastic bands, etc. The bridge between the glasses are made telescopic, etc. permitting changes in the inter-pupillary distance for the babies, children, adults, and animals, etc. (see FIG. 15).

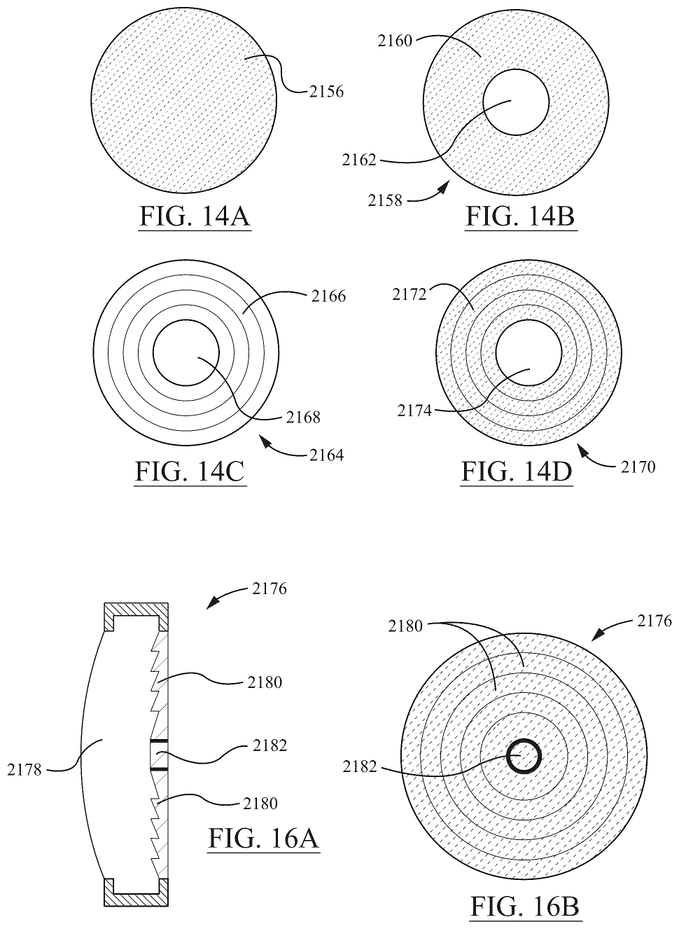

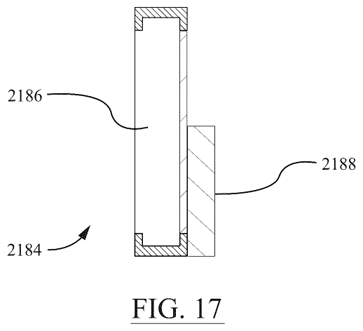

In another embodiment, with reference to FIGS. 16A and 16B, a hybrid fluidic lens is provided with one or more transparent plates. In FIG. 16A, a side view of the hybrid fluidic lens with the one or more transparent plates is illustrated. The thin back plate in FIG. 16A is a diffractive plate or a lens which has a hole in its center to permit the patient to see through it and see any object in focus. Because the back plate has a pinhole, every object viewed through a pin hole by the patient becomes in focus. In this embodiment, the edges of the pinhole are black so as to prevent light scattering from the edges of the hole. The diffractive plate (or lens) can have or not have transitional pigment that darkens by the absorption of light, specifically ultraviolet (UV) light. As shown in the top view of FIG. 16B, the rest of the back plate may be any diffractive plate with multi-zones providing focal points for any distance from far to near for the eye. Therefore, this hybrid lens acts as a multifocal lens while the distance power is corrected by the fluidic membrane (plus or minus) and the diffractive lens and the pinhole or transitional pinhole lens provide focus for any distance from the far to near for the human patient or an animal. The front plate of this lens in FIG. 16A also may be used to correct astigmatic aberration at any axis. In FIG. 17, an additional version of the hybrid lens is depicted, wherein the back plate of lens can have additional plus lenses for presbyopia correction.

An illustrative embodiment of a diffractive fluidic lens 2176 having a front fluidic lens chamber 2178 with a flexible membrane and a rear Fresnel diffractive lens 2180 with multiple zones of prisms to provide many fixed diffractive plus zone focal points is depicted in FIGS. 16A and 16B. As shown in FIG. 16A, the flexible membrane is supported in an outer housing with a Fresnel diffractive lens 2180 forming the back of the housing. The Fresnel diffractive lens 2180 has a central pinhole aperture region 2182 (see FIGS. 16A and 16B) that may have a diameter between 1.2 and 4.0 millimeters. The flexible membrane and the outer housing together define an internal fluid chamber 2178 for receiving a fluid therein (e.g., a laser fluid with a high index of refraction). A fluid reservoir may be fluidly coupled to the fluid chamber 2178 of the fluidic lens 2176 so that the fluid may be injected into, or withdrawn from the fluid chamber 2178 by means of a fluid pump. A top view of the rear Fresnel diffractive lens 2180 of the diffractive fluidic lens 2176 is shown in FIG. 16B.

An illustrative embodiment of a presbyopic fluidic lens 2184 with a flexible membrane front lens and an additional rear solid lens 2188 of +1.00 D to +3.00 D is depicted in FIG. 17. As shown in this figure, the flexible membrane of the fluidic front lens is supported in an outer housing with a solid flat glass plate forming the back of the housing. The flexible membrane and the outer housing together define an internal fluid chamber 2186 for receiving a fluid therein (e.g., a laser fluid with a high index of refraction). A fluid reservoir may be fluidly coupled to the fluid chamber 2186 of the presbyopic fluidic lens 2184 so that the fluid may be injected into, or withdrawn from the fluid chamber 2186 by means of a fluid pump.

In one embodiment (e.g., for animals), the frames can be made with leather to fit over the side and brow of the eyes, thereby preventing the peripheral light to cause glare for the animal or human, etc., while keeping the cornea moist to prevent it from drying out. The frame may be fabricated such that the inventive lens can easily be inserted into (i.e., “pop into”) and be removed from (“pop out”) the frame.

In one embodiment, these lenses are made for the albinism patients who are always bothered from the side glare form outside, since their irises do not have pigment to form a barrier for the light entering their eyes, or in patients who have lost part of their iris after trauma.

In one embodiment, the hybrid fluidic and diffractive lens can be an intraocular lens.

In one embodiment, since the image of the right eye and left eye for a given distance are in focus, the brain can convert them into stereovision without the need for too much convergence of the eyes that can cause headache.

In one embodiment, the hybrid lens is used for a microscope.

In one embodiment, the hybrid lens is used for an operating microscope.

In one embodiment, the hybrid lens is used for a camera.

In one embodiment, the hybrid lens is used for a light field camera.

In one embodiment, the hybrid lens is used for VR or AR goggles.

In one embodiment, the hybrid lens is used for the ordinary glasses for babies or adults.

In one embodiment, the hybrid lens is used for patients who have lost their crystalline lens after traumatic eye injuries.

In one embodiment, the hybrid lens is used for a telescopic system.

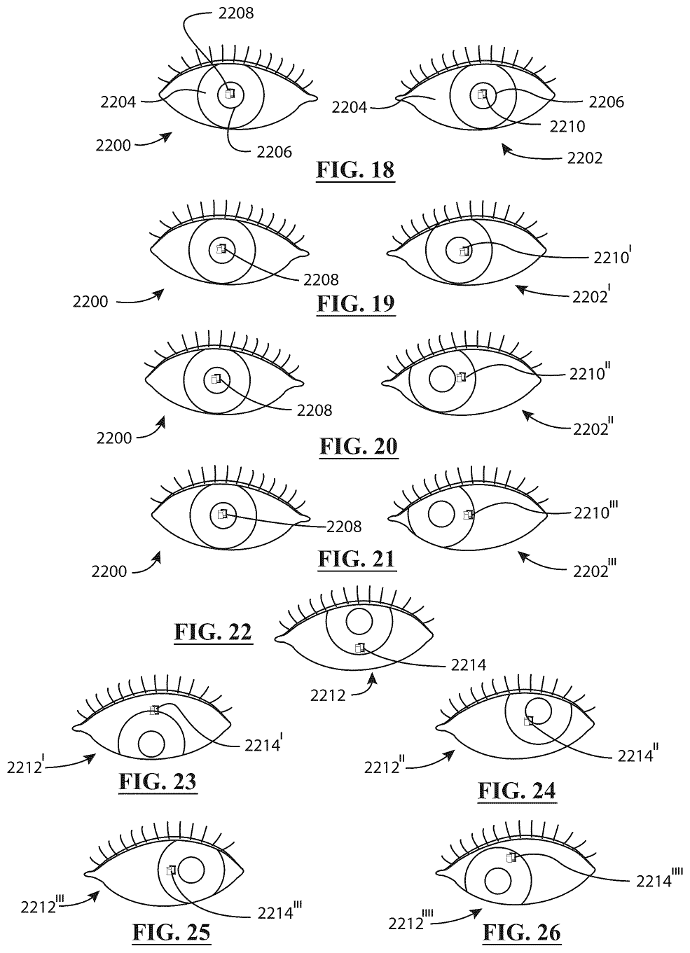

In one embodiment, the binocular deviation of the patient's eye is examined by asking the patient to look at a light source located at a near distance that requires both eyes to converge at a light source while they are photographed, where the light source creates a light reflex on the person's cornea which is photographed. If the eyes have no deviation, the light reflex is located at the central part of the corneas, whereas if one or the other eye deviates it indicates the presence of a phoria, such as esophoria or exophoria or vertical phoria or an oblique phoria. The degree of the light deviation can be measured by the distance that the light reflex is deviated from the center and the direction of deviation is recognized by the location of the light reflex seen on the cornea or adjacent structures (see e.g., FIGS. 18-26). The distance of the light reflex to the center of the cornea measured in millimeters (mm) is equal to the prismatic deviation where one millimeter (mm) is equal to 15 PD and 4 mm is equal to 60 PD.

For example, FIG. 18 is a front view of right and left eyes 2200, 2202 of a patient without any phoria condition. In the right eye 2200 of FIG. 18, which includes the iris 2204 and pupil 2206, the light reflex 2208 is centered in the pupil 2206. Similarly, in the left eye 2202 of FIG. 18, which includes the iris 2204 and pupil 2206, the light reflex 2210 is centered in the pupil 2206. In FIG. 19, the light reflex 2208 is centered in the pupil of the right eye 2200, but the light reflex 2210′ of the left eye 2202′ has an off-center deviation of 1 mm. In FIG. 20, the light reflex 2208 is centered in the pupil of the right eye 2200, but the light reflex 2210″ of the left eye 2202″ has an off-center deviation of 2 mm. In FIG. 21, the light reflex 2208 is centered in the pupil of the right eye 2200, but the light reflex 2210′″ of the left eye 2202″″ has an off-center deviation of 3 mm. In FIG. 22, the left eye 2212 has a hyperphoria condition where the light reflex 2214 is below the pupil of the eye 2212. In FIG. 23, the left eye 2212′ has a hypophoria condition where the light reflex 2214′ is above the pupil of the eye 2212′. In FIG. 24, the left eye 2212″ has an oblique hyperphoria condition where the light reflex 2214″ is below and to the side of the pupil of the eye 2212″. In FIG. 25, the left eye 2212″″′ has an exophoria condition where the light reflex 2214″′ is to one side of the pupil of the eye 2212″ ″. In FIG. 26, the left eye 2212″″″″ has an oblique hypophoria condition where the light reflex 2214″″′ is above and to one side of the pupil of the eye 2212″″.

In one embodiment, the patient's eye phorias are corrected with one or two tunable prism positions in front of the eye by artificial intelligence (AI) software that controls the degree of fluid that enters or exits from the tunable phoropter and also recorded is the degree of phoria and the amount of prism diopter (PD) required for its correction to see with both eyes at any distance from the eye for reading automatically via a software or intermediated distances prior to checking the vision and its refractive error (see e.g., FIGS. 28 and 29). As described above, the tunable prisms can be formed by deformable balloons with a tube for inflating or deflating the balloons. Alternatively, the balloons in the tunable prisms can be replaced with a transparent elastic polymeric material, such as silicone or other compressible or deformable elastic, transparent materials or polymers that permit any wavelength of light from UV to infrared and beyond to pass through it, as needed. When a transparent elastic polymeric material is used in the tunable prisms rather than the balloon, the tube is not required.

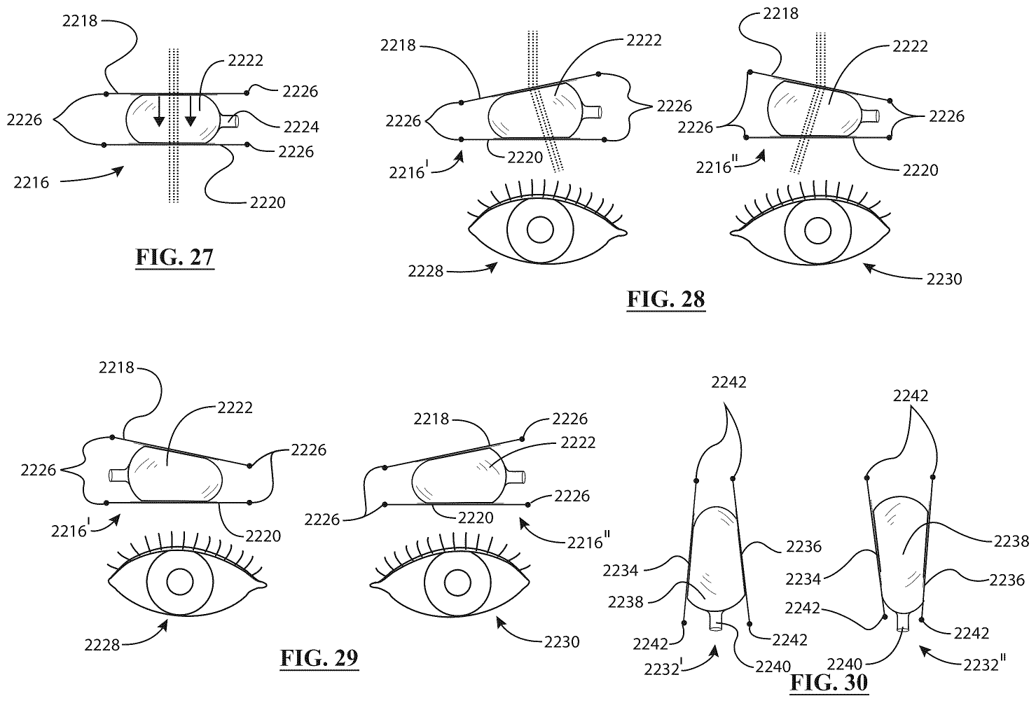

In FIGS. 27-29, an illustrative embodiment of a tunable prism 2216 with a first movable transparent plate 2218 and a second stationary transparent plate 2220 is depicted. In the embodiment of FIGS. 27-29, the first movable transparent plate 2218 is separated from the second stationary transparent plate 2220 by a transparent balloon 2222. The transparent balloon 2222 may be made of silicone or another transparent elastic polymer, and can be filled with a fluid (e.g., water or other transparent liquid) via a tube 2224 that can be connected to a pump. In the embodiment of FIGS. 27-29, the plates 2218, 2220 are provided with selectively activatable electromagnets 2226. In FIG. 27, the electromagnets 2226 are not activated, and the plates 2218, 2220 are disposed parallel to one another. In FIG. 28, a pair of tunable prisms 2216′, 2216″ are in a first vision correction configuration, where the outer electromagnets 2226 on the tunable prisms 2216′, 2216″ are magnetically activated so as to create a base-in prism for correcting a phoria of the eyes 2228, 2230. In FIG. 29, the pair of tunable prisms 2216′, 2216″ are in a second vision correction configuration, where the inner electromagnets 2226 on the tunable prisms 2216′, 2216″ are magnetically activated so as to create a base-out prism for correcting a phoria of the eyes 2228, 2230.

In FIG. 30, an illustrative embodiment of a pair of vertically activated tunable prisms 2232′, 2232″ is depicted. Each vertically activated tunable prism 2232′, 2232″ has a first movable transparent plate 2234 and a second stationary transparent plate 2236. In the embodiment of FIG. 30, the first movable transparent plate 2234 is separated from the second stationary transparent plate 2236 by a transparent balloon 2238. The transparent balloon 2238 may be made of silicone or another transparent elastic polymer, and can be filled with a fluid (e.g., water or other transparent liquid) via a tube 2240 that can be connected to a pump. In the embodiment of FIG. 30, the plates 2234, 2236 are provided with selectively activatable electromagnets 2242. Each of the tunable prisms 2232′, 2232″ is magnetically activated in a different vision correction configuration.

In one embodiment, one can add the prismatic correction to the patient's glasses as a single prism or as Fresnel prism or this degree of prismatic deviation is either added to the glasses or lenses inside AR or VR goggles ahead of time.

In one embodiment, the wall of the pinhole in the lens used for the prismatic lenses is darkened to prevent light reflection of the pinhole that is a through hole.

In one embodiment, the pinhole is a mask with a hole in it that is placed in the center of the surface of the prismatic lenses to compensate for refractive errors, such as in near vision or reducing the astigmatic correction of the lens.

In one embodiment, the adjustable prisms can be used in combination with software for other applications in different industries, such as for cars, other vehicles, security systems, military applications, robotics, drones in any cameras, microscopy, machine vision, or directing the light to obtain a stereopsis image from a subject at any wavelength from infrared and beyond to ultraviolet, UVA, UVB, UVC radiation, etc.

In one embodiment, one can add the tunable prismatic correction to the patient glasses as a single prism or two prisms for both eyes and correct for any degree of prismatic deviation or any direction of deviation (see e.g., FIGS. 28 and 29) so that both eyes can converge and see stereoscopically for any given distance from the eye, and the correction can be done mechanically or electronically activated by software.

In one embodiment, one uses two tunable prisms, one for each eye to compensate for the complex variation of each eye's deviation independently to be able to focus two images by the activation of their magnets via software and to overlay the image, or with angular separation of each eye creating potentially a stereovision for a person, or for a camera, or one or two fluidic light field cameras positioned right and left of each other with a wide angle view as a security system having close to 360 degrees or less field of information providing perfect pixelated images that can be seen individually or collectively. The images can also be analyzed by software to access the degree of separation of various outside objects in their field in 2D or 3D format by oscillating the prisms without the need for rotating the camera itself.

In one embodiment, two cameras and their tunable prisms are positioned side by side and the tunable prisms are electronically activated with software to oscillate at desired direction(s), obtaining a perfect stereo image for >200 or more degrees field of view and analyzed by artificial intelligence (AI) software. This system has applications in medicine, diagnostics, industry, security systems, and the military (e.g., in drones, missiles, or planes).

In one embodiment, the camera is a hyperspectral or multispectral camera for the analysis of the images in a more detailed manner in ophthalmology, medicine dermatology, etc., having artificial intelligence (AI) software for disease diagnosis.

In one embodiment, software can control the motion of the prism very rapidly to scan not only an outside object but also its surrounding field of view, creating thereby two or three dimensional images as needed or providing various information about the position, characteristics, and direction of an object which can be useful for a patient, but also useful in security, surveillance, or in military operations, etc.

In one embodiment, multiple tunable prisms can work together, activating various areas of the prism via software to create the best possible stereovision for a person or patient or an object or landscape, etc. for a camera(s), which is useful for a stable or moving drone photographing and transmitting the information via the internet to a desired system(s) for instant analysis or image reformation in real-time.

In one embodiment, two cameras side by side with tunable prisms in front of them can easily focus on an object for machine vision or security systems with artificial intelligence (AI) software, creating stereo images of an object for precision robotic vision for recognition of an object, or e.g., human or a device for its recognition and control of the arm(s) of a robot or even precision robotic surgery by the AI software, or in industry along with a laser to build an object or do cosmetic surgery or for military applications with AI software or to be used alone, or in drones for precision flight recognition or aiming a laser, e.g., on a missile, or avoiding colliding with an object in its flight or use with a laser in precision military applications, or for a car to avoid collisions with its AI software, etc. or automatic inspection and process control in industry, e.g., pharmaceutical industry via AI software control, or in security systems with facial recognition software to recognize a person, etc.

In one embodiment, the tunable prisms can be used with a person's glasses, e.g., in children to correct an abnormal eye's deviation, such as in strabismus, esophoria, exophoria, hyperphoria, hypophoria, or oblique deviation of one or both eyes (see e.g., FIGS. 18-26) to assist in proper view to focus the eyes on a near object or on a far object where the AI software automatically controls the prism by increasing or decreasing the prism diopter (PD) or direction of the deviation where the electronics and batteries are positioned on the arms of the glasses.

In one embodiment, the rotation of the tunable prism can be made to coordinate with the motion of the eye toward a specific direction, etc. or be remotely controlled by the person carrying it or in robotic vision.

In one embodiment, the tunable prisms can be used in driverless cars, etc. The tunable prisms can also scan rapidly to control direction of motion of a car, train, plane, etc. and avoid collisions, or in military to induce a precision collision if needed.

In one embodiment of a tunable prism, the moveable plate can have if needed a Fresnel prism that enhances the effect of deviation towards a specific direction.

In one embodiment for correction of convergence, the tunable prisms can assist the eye to reduce the deviation of an eye gradually when the ocular muscles are getting stronger so that a person or a child can be weaned off the eye from carrying a prismatic correction gradually, e.g., in children, or the tunable prism can be used by a patient to strengthen by exercise a specific ocular muscle and move them repeatedly toward a certain direction.

In one embodiment, the tunable prism is used for a person, or for a digital camera, to work like a human eye which micro-oscillates 1-10 Hz or more back and forth to stimulate the retina at its focal point, the fovea, thereby creating a better stimulus for the brain or the digital camera to image sharply the structure of an object. Because the present digital cameras are not made to create an oscillation, the use of a tunable prism that can oscillate by electronic stimulation permits any degree of frequency of oscillation to provide sharp in-focus images to be analyzed by its artificial intelligence (AI) software and average it out for the best sharp image formed digitally with an oscillating prism of 1 Hz-20 KHz frequencies by simultaneous activation of both the tunable camera (refer to U.S. Pat. No. 10,606,066, the disclosure of which is incorporated by reference herein in its entirety) and tunable prism where the data obtained is far more pixelated, providing a better resolution than standard cameras, the oscillating tunable prism in combination with the tunable fluidic light field camera (in U.S. U.S. Pat. No. 10,606,066) produce a sharp focus for any stable or moving object located at any distance from the camera and analyzed fast with neuromorphic or subtraction software of a dynamic facial recognition (e.g., refer to U.S. Pat. No. 11,309,081, the disclosure of which is incorporated by reference herein in its entirety) that would have not been possible previously with a motionless camera. In the current system, the fluidic light field camera provides an in-focus focal point of any object in front of this digital camera and the oscillation of the tunable prism in front of the camera makes the image sharper by its artificial intelligence (AI) software providing rapidly an image with higher resolution.

In one embodiment, a flying drone, an airplane, or a satellite equipped with a combination of a motion detection system, an AI software, and an electronically-induced oscillating prism and fluidic light field camera can image and trace another moveable object, an animal, a human, a car, a train, a boat, a storm, a hurricane, a bullet, a missile, or an earthquake, the direction of motion, and the frequencies of its oscillation, such as waves of the ocean, the ground in an earthquake, or wind-induced motion on the ground, or the speed of the motion, the frequency of an object's motion, time, and direction that is taken and evaluated with AI software of dynamic facial recognition (see U.S. Pat. No. 11,309,081) or neuromorphic software and communicated to another system via the internet, thus creating not only 3-D images, but also a predictive value for a time-traveled object to reach another location or a predictive value for a disease process.

In one embodiment, the modified AR or VR with AI software, a tunable camera, and tunable prism (e.g., refer to U.S. Pat. No. 11,372,230, the disclosure of which is incorporated by reference herein in its entirety) are used for home diagnosis of an eye disease and bot-assisted artificial intelligence (AI) is used to ask questions and/or respond to the patient's questions to shorten the exam time by limiting the areas of interest for measuring and refinement of visual acuity and follow up of the eye diseases and recognition of the ocular pathology and their changes over a time period involving the cornea, lens, vitreous gel, retina and its vasculature, and optic nerve head, and communicating with the patient and the doctor, etc. In this embodiment, the bot-assisted AI asks questions or responds to the patient's questions to limit the potential of eye diseases involved, and thereby shorten the exam time of the patient.

In one embodiment, augmented intelligence AR or VR with a phoropter camera (see U.S. Pat. Nos. 9,016,860 and 9,191,568) are used for diagnosis of ocular diseases or as a home monitoring device in diabetic patients with diabetic retinopathy or diabetic macular edema, age related macular degeneration, retinal vascular diseases, by using the collimated light that enters the eye through a prismatic lens in front of the eye to reach the retina where the reflected light from the retina, vitreous and lens and cornea passes through a dichroic mirror which diverts the light from the eye to a camera that records the images of the retina, vitreous, lens, cornea, and the images are analyzed with augmented intelligence or bot-assisted artificial intelligence (AI) software to rapidly diagnose a disease or its stage in a diseased cornea, lens, vitreous, or retina and optic nerve, then the analyzed images are transmitted via the internet to the patient and his or her ophthalmologist or optometrist along with the refractive errors corrected from the tunable lenses and corrected values obtained by the tunable prisms' software for bilateral vision.

In one embodiment, the fluidic camera or the phoropter (see U.S. Pat. No. 9,191,568) is equipped with dynamic facial recognition software and optical coherence tomography (OCT) and bot-assisted artificial intelligence (AI) software used for home monitoring by imaging where the cornea, lens, vitreous, and retinal images of the patient is scanned rapidly with the fluidic lens camera and its dynamic imaging AI software or a neuromorphic camera records rapidly the dynamic changes of a structure(s) and analyzes them with AI software and the information is immediately transmitted to a doctor to confirm the diagnosis of a disease, such as diabetic macular edema, degree of the sub-retinal fluid, or the existence or the progression of an age-related macular degeneration or a central vein occlusion, or branch vein or artery occlusion, or retinitis pigmentosa, or presence or absence of a tumor or optic nerve head edema, or changes due to glaucoma or the retina in diabetic retinopathy or change in the peripapillary micro-vasculatures, the retinal thickness, or cellular changes in the retina or choroid, etc.

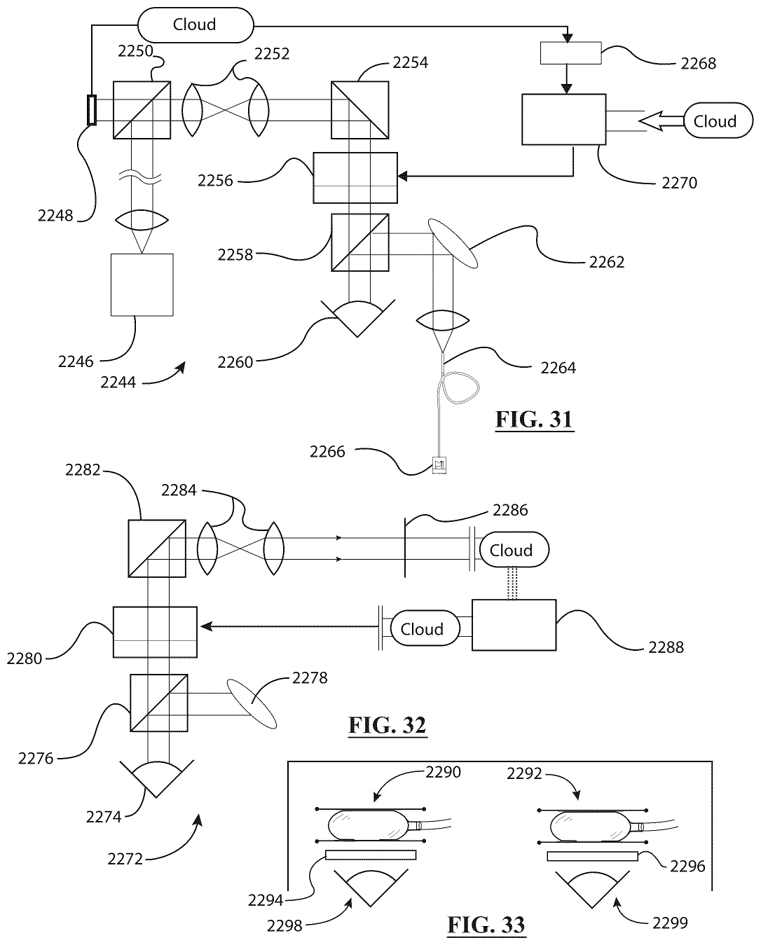

In one embodiment, with reference to FIG. 31, the fluidic phoropter camera (see U.S. Pat. Nos. 9,016,860 and 9,191,568) can automatically focus the beam on the patient's retina to photograph the retina, and the Shack Hartmann sensor of the unit can be connected to the basic unit or it can be connected to the unit via the internet, thereby making the unit portable and useable as a home monitoring system to follow a patient or evaluate a new patient for his or her refractive error or an ocular disease. As the illustration in FIG. 31 indicates, while the patient is observing a visual display, an infrared beam enters an activated rapidly oscillating tunable prism in front of the pupil, scanning the cornea, lens, retina and returns back passing through the fluidic lenses and is diverted via a prismatic beam splitter (PBS) and via a relay lens toward another prismatic beam splitter either toward a Shack-Hartmann sensor or a camera. Here the activated sensor's software can directly correct the fluidic lenses to correct the optical aberration of the eye or a sensor can send the signal through the cloud to another remotely located sensor that can activate fluidic lenses via the cloud and AI remotely in order to activate the pumps to modify the fluidic lenses' shape which corrects the refractive errors of the eye while seeing the visual display (e.g., with a fixation chart or an object). Similarly, the light that is diverted to the oscillating tunable prism and fluidic camera can directly transmit the scanned image information to the software of its digital camera or as above the in-focus of scanned images (signals) of the person's retina, lens, and cornea is sent to the cloud located elsewhere to be imaged and the AI software recognizes the patient if the patient has been photographed and the images of the scanned cornea, lens, and the retina, etc. are analyzed with bot-assisted AI software to recognize the patient, and a disease process and the diagnostic information/images are transmitted via the internet to the patient or his or her doctor.

In one embodiment, a visible light or a beam used for a multispectral or hyperspectral camera is sent to the eye through the same pathway after the refractive errors of the eye are corrected with fluidic lenses, so that the retina is in focus for photography of the cornea, lens, and retina to be analyzed with bot-assisted AI software.

In another embodiment, the system and camera described above has an optical coherence tomography unit (see U.S. Pat. No. 9,191,568) that produces a near-infrared beam that is used for the patient's ocular imaging where the camera can be attached to the unit equipped with bot-assisted AI software to analyze the curvature of the cornea, its transparency, and/or the density or cloudiness of the crystalline lens for the degree of the cataract formation or analyze the structures of the retina or the optic nerve with a very high resolution, since the optical aberrations of the eye have been corrected by the fluidic lenses initially.

In one embodiment, the system can provide the information of the optical aberration of the eye simultaneously with images of the cornea, lens, retina, etc. to be analyzed with bot-assisted AI software for the presence or absence of a disease process, such as retinitis pigmentosa, age-related macular degeneration, diabetic retinopathy, macular edema, etc.

In one embodiment, with reference to FIG. 32, the light enters the eye and exits after passing through the fluidic lenses to a sensor that sends the information through the cloud to a Shack-Hartmann system and with bot-assisted AI software located elsewhere. After analyzing the information with AI, the signals are sent back via the cloud to the first unit located in the initial place with the unit to activate the pump of the fluidic lenses and correct the refractive error of a patient while he or she is looking at a visual display in the first location, then the tunable fluidic camera (U.S. Pat. No. 10,606,066) obtains images from the retina through its activated oscillating or scanning tunable prism and the images and diagnosis are transmitted via cloud computing to the patient's smartphone and his doctor as is done with any smartphone camera, thereby simplifying home monitoring the disease process of the cornea, lens, and the retina.

In another embodiment, FIG. 33 illustrates that because of the size, thickness, and weight (see U.S. Pat. No. 11,372,230) of the tunable prisms, fluidic lenses, and the imaging camera, the Shack-Hartmann sensors and a bot, the standard goggles or glasses are converted to a larger version that made the goggles too bulky and heavy to carry them on the nose, therefore the enlarged modified AR or VR system is placed in front of the eye on a small portable table or a small AR or VR kiosk model, or for use as home monitoring device where the unit is kept, the tunable prism, fluidic lenses, the bot, the camera and a small-sized computer or a chip, however the Shack-Hartman sensor with or without fluidic lenses and electronics and bot-assisted AI, AR, VR software and with dynamic facial recognition software (see e.g., U.S. Pat. No. 11,309,081) are reached with cloud computing with two-way communication back to the unit, where this binocular system evaluates simultaneously the stercovision, using an OCT for imaging the cornea, lens, vitreous, and retinal pathology in various ophthalmic and systemic diseases, including the function of oculomotor system affecting convergence and accommodation, etc.

In one embodiment, the system described in U.S. Pat. No. 9,191,568 is used with bot-assisted AI software to correct a refractive error of the person's eye with fluidic lenses and a Shack-Hartmann system and AI software of the tunable lenses to correct the refractive error of the eye for seeing an object or a video stream presented to the eye and the use of tunable prism software to correct the convergence or divergence deficiencies, etc. of the eyes, thereby creating in-focus virtual images of each eye analyzed with bot-assisted AI or virtual reality software (e.g., metaverse software) and presented to one or both eyes.

In one embodiment, a video stream can be obtained from in-focus images, analyzed using with bot-assisted AI software to produce an image of each cornea, lens, vitreous, or the retina and optic nerve to diagnose disease involving the ocular structures, such as the cornea, lens, vitreous, and retina and optic nerve head, while correcting the refractive error of the eyes for sharp in-focus vision, etc. This embodiment may further include analyzing the entire information with augmented intelligence (AI) software, which can be communicated to the patient's smartphone and to his or her ophthalmologist or optometrist, or general practitioner via the internet for the confirmation of presence or lack of a disease process. Also, dynamic facial recognition software, which confirms the patient's identity while presenting the changes in between the past and present images, may be used as part of this embodiment.

In one embodiment of the fluidic lens camera described in U.S. Pat. No. 9,191,568 alone or combined with the AR or VR and used with the with bot-assisted AI software to assist in providing the health information, and in diagnosing the ocular pathology or systemic diseases that affect the retina, such as hypertension, diabetes, Alzheimer's Disease, age-related macular degeneration (ARMD), genetic diseases affecting the retina, such as retinitis pigmentosa, Stargardt's syndrome, Best dystrophy, etc., inflammatory diseases of the retina, such as toxoplasmosis, viral or fungal retinitis, etc., existence of an ocular tumor, such as retinoblastoma or brain tumor causing the optic nerve swelling or glaucoma optic nerve cupping, retinopathy of prematurity, etc., a cataract, etc. or a corneal disease, such as keratoconus, corneal dystrophies, Fuchs dystrophy, etc. using AI software as medical information grows beyond one's capabilities of any human to know everything or remember them instantaneously to take care of a patient, remote bot-assisted AI analysis can contribute to the health in developing countries or places where there may not be a doctor since the system can be modulated where the information can be obtained in one place and the diagnosis is done in another place with AI software. In these cases, the obtained health information is printed out or sent via the internet to the patient's doctor or ophthalmologist or optometrist, internist, etc. to be validated without bias toward the patient's care.

In one embodiment, the virtual information obtained after the light exiting from the eye and sent to a camera, such as a light field camera, a morphometric camera, or MicroCalibir with long-wave-IR (LWIR) camera where the images are recorded and analyzed by the AI software of the camera, and the images are sent back to the physician or to the patient's corresponding eyes having AR or VR specific goggles software (see U.S. Pat. No. 11,372,230), permitting the patient or a person to see his or her virtual image of his or her own corneas, lenses, vitreous, or the retinas, etc., and to appreciate the normal structure or pathological structures of his or her own eye by looking at his or her virtual images, etc., these images can be compared later if the patient has been treated for a disease or using the subtraction software described in dynamic facial recognition software that analyzes the differences of images or motion-induced changes in <1 millisecond (see U.S. Pat. No. 11,309,081) and with neuromorphic cameras and AI software presenting the dynamic changes toward an improvement or worsening of the condition. Also, dynamic facial recognition software, which confirms the patient's identity while presenting the changes in between the past and present images, may be used as part of this embodiment.

In one embodiment, the camera or Shack-Hartmann or both systems can be located elsewhere away from the basic phoropter, communicating using two-way cloud computing with the tunable lenses, visual display/object, and tunable prism.

In one embodiment, all of the fluidic lenses and Shack-Hartmann system can be replaced with a light field camera or preferably a Peyman light field camera (see e.g., U.S. Pat. No. 10,606,066) with its software and algorithm with or without a bot and present fast in-focus images of an external object such as cornea, lens, vitreous, and retina at any point and differentiate the normal structure from the diseased structure with its computer and AI software and recall virtual 2-D or 3-D images at any point in the cornea, lens, vitreous, and retina.

In one embodiment, all of the fluidic lenses and Shack-Hartmann system can be replaced with a light field camera or preferably a Peyman light field camera (U.S. Pat. No. 10,606,066) with its software and algorithm with or without a bot and present fast in-focus images of an external object such as cornea, lens, vitreous, and retina at any point and differentiate the normal structure from diseased structure with its computer and AI software and recall virtual 2-D or 3-D images at any point in the cornea, lens, vitreous, and retina where the system is combined with a slightly larger modified AR or VR goggles for in home diagnostics communicating with a smartphone and/or computer to the patient or his or her doctor via the internet.

In one embodiment, the smartphone can be combined with a small Peyman light field camera replacing the presently available camera that produces only 2-D in-focused images, thereby producing 3-D images of any object in its field of view and when combined with dynamic facial recognition can recognize any object or moving object in its field.

In one embodiment, the light field camera or Peyman light field camera can be equipped with an infrared LED or laser for night vision photography in the dark providing sharp IR images of an object, human, animal, or potentially from structures inside the body's cavity, such as eye, cornea, lens, and retina, etc. that can be reached with an IR beam.

In one embodiment, the smartphone can be combined with a small Peyman light field camera with oscillating tunable prism (see e.g., U.S. Pat. No. 11,372,230) for light or IR wide angle imaging and scanning a wide field of view with its use for a security system, a military application, or in medicine, physical activity, etc. and the obtained images or video can be analyzed with AI software, transmitted via cloud computing to any desired place and/or can be encrypted prior to sending the information (images) out, this system also can be combined with a bot for recording sound or words information, etc., and may be combined with dynamic face recognition, etc. (see e.g., U.S. Pat. No. 11,309,081).

In one embodiment, a Peyman light field camera with an oscillating tunable prism acts as a device to create a wide angle view of images from the outside world in a 3-D manner.

In another embodiment, the oscillation prismatic lens can project the light in the eye of a person to divert the incoming light, to the peripheral field of the retina, thereby acting like a scanner and the light that returns from the eye can be collected in another camera via a dichroic mirror producing wide angle images from the retina, the lens, and the cornea on its way out that could be recorded on an external camera via a dichroic mirror.

In one embodiment, the oscillation prismatic lens of the camera is associated with a smartphone having a bot that can communicate with the patient before the pictures are taken and the communication is recorded and an AI system with or without AR or VR, can diagnose the characteristics of images and provide a diagnosis or the potential disease abnormalities at various stages or compare it with previous existing images of the patient and the data is communicated to the patient and cloud computing to the patient's doctor with or without recommended potential therapy.

In one embodiment, the oscillating prism's outer surface could have a convex or concave surface producing a smaller field or a larger field from the retina or a much wider field from the retina depending on its surface concavity or convexity, since the tunable prism oscillating back and forth and scans different areas of the field of view or, e.g., inside the eye's retina and simultaneously images, with no need for the lens to touch the cornea to provide a wide angle view of the retina or create a wide angle view from the OCT, multispectral, or hyperspectral images of the retina, lens, or cornea for analysis with the AI system of the retinal structures or the lens, e.g., in a patient with a cataract, and the cornea, such as in diagnosis of a keratoconus, etc., and communicate with the patient and his or her doctor via a bot via cloud computing. The bot simplified the computing by providing a history of the patients complaints, etc.

In one embodiment, the combination of the tunable prism with its central front circular opening creates a convex lens that focuses the light behind the pupil and spreads out, as a result, the returning light from the retina will have a view of >180 degree from the retina that can be captured by a digital camera, a light field camera, or a Peyman light field camera (see e.g., U.S. Pat. No. 10,606,066), and the image is recreated by the camera's sensors and sent to its computer software without the need for a fluidic lens system or Shack-Hartmann's sensor to bring the images in focus making the system lighter and the images can be transmitted electronically as with a smartphone to the cloud so as to be retrieved, and sent to a doctor or a patient as needed to provide a diagnosis using artificial intelligence (AI) or machine learning software, etc.

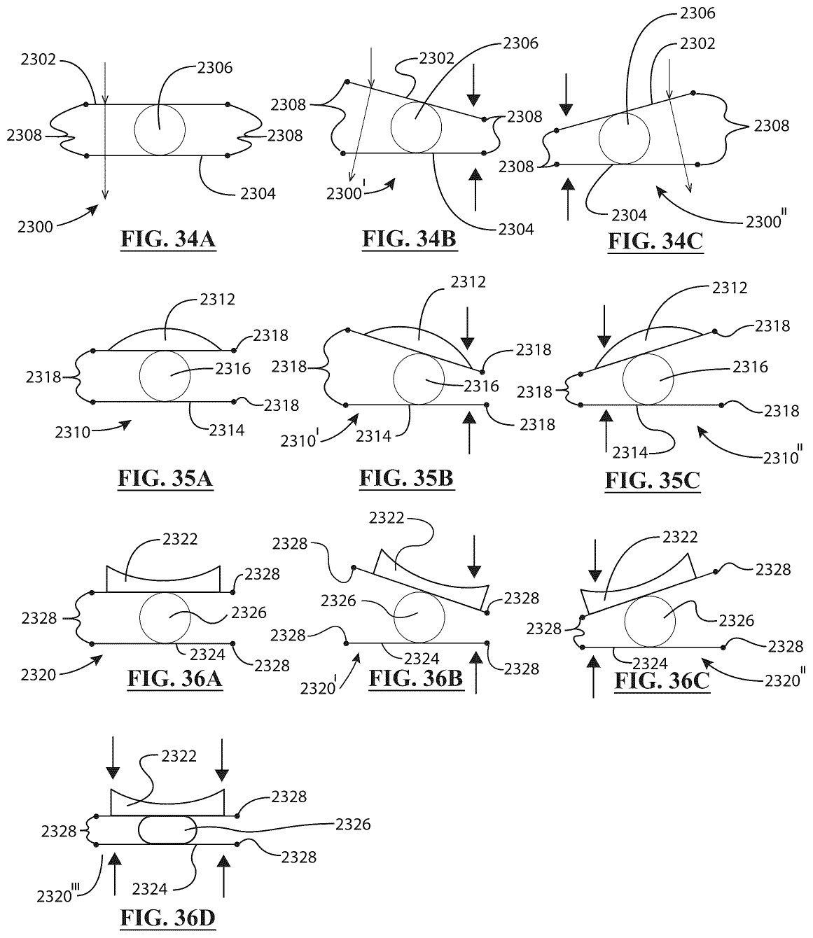

In one embodiment, the transparent surfaces of the prism on which a balloon or a refillable transparent bag is mounted may have many different shapes that can affect the way light refracts from it (see FIGS. 34A-40).

In one embodiment, the amount of the fluid in the balloon can affect the shape or degree of the prismatic effect by separating the two plates that are connected with a joint (see e.g., the tunable prisms in FIGS. 39A, 39B, and 40).

In one embodiment, the prismatic plate is magnetically stimulated (see e.g., FIGS. 34A-38B).

In one embodiment, the surface of the transparent plate can be flat, convex, or concave affecting the direction of light passing through the plate (see e.g., FIGS. 34A-38B).

In FIGS. 34A-34C, an illustrative embodiment of a tunable prism 2300 with a first movable transparent plate 2302 and a second stationary transparent plate 2304 is depicted. In the embodiment of FIGS. 34A-34C, the first movable transparent plate 2302 is separated from the second stationary transparent plate 2304 by a transparent flexible polymeric ball 2306. The transparent flexible polymeric ball 2306 may be made of silicone or another transparent elastic polymer. In the embodiment of FIGS. 34A-34C, the plates 2302, 2304 are provided with selectively activatable electromagnets 2308. In FIG. 34A, the electromagnets 2308 are not activated, and the plates 2302, 2304 are disposed parallel to one another. In FIG. 34B, the electromagnets 2308 on a first side are activated such the plates 2302, 2304 of the tunable prism 2300′ are disposed in a first vision correction configuration. In FIG. 34C, the electromagnets 2308 on a second side are activated such the plates 2302, 2304 of the tunable prism 2300″ are disposed in a second vision correction configuration.

In FIGS. 35A-35C, an illustrative embodiment of a tunable prism 2310 with a first movable transparent convex plate 2312 and a second stationary transparent plate 2314 is depicted. In the embodiment of FIGS. 35A-35C, the first movable transparent convex plate 2312 is separated from the second stationary transparent plate 2314 by a transparent flexible polymeric ball 2316. The transparent flexible polymeric ball 2316 may be made of silicone or another transparent elastic polymer. The first movable transparent convex plate 2312 and the second stationary transparent plate 2314 may be made from a transparent glass or plastic material. In the embodiment of FIGS. 35A-35C, the plates 2312, 2314 are provided with selectively activatable electromagnets 2318. In FIG. 35A, the electromagnets 2318 are not activated, and the plates 2312, 2314 are disposed parallel to one another. In FIG. 35B, the electromagnets 2318 on a first side are activated such the plates 2312, 2314 of the tunable prism 2310′ are disposed in a first vision correction configuration. In FIG. 35C, the electromagnets 2318 on a second side are activated such the plates 2312, 2314 of the tunable prism 2310″ are disposed in a second vision correction configuration.

In FIGS. 36A-36D, an illustrative embodiment of a tunable prism 2320 with a first movable transparent concave plate 2322 and a second stationary transparent plate 2324 is depicted. In the embodiment of FIGS. 36A-36D, the first movable transparent concave plate 2322 is separated from the second stationary transparent plate 2324 by a transparent flexible polymeric ball 2326. The transparent flexible polymeric ball 2326 may be made of silicone or another transparent elastic polymer. The first movable transparent concave plate 2322 and the second stationary transparent plate 2324 may be made from a transparent glass or plastic material. In the embodiment of FIGS. 36A-36D, the plates 2322, 2324 are provided with selectively activatable electromagnets 2328. In FIG. 36A, the electromagnets 2328 are not activated, and the plates 2322, 2324 are disposed parallel to one another. In FIG. 36B, the electromagnets 2328 on a first side are activated such the plates 2322, 2324 of the tunable prism 2320′ are disposed in a first vision correction configuration. In FIG. 36C, the electromagnets 2328 on a second side are activated such the plates 2322, 2324 of the tunable prism 2320″ are disposed in a second vision correction configuration. In FIG. 36D, the electromagnets 2328 on both sides are activated such the plates 2322, 2324 of the tunable prism 2320′″ are disposed in a third vision correction configuration where the transparent flexible polymeric ball 2326 is evenly compressed and the plates 2322, 2324 remain parallel to one another.

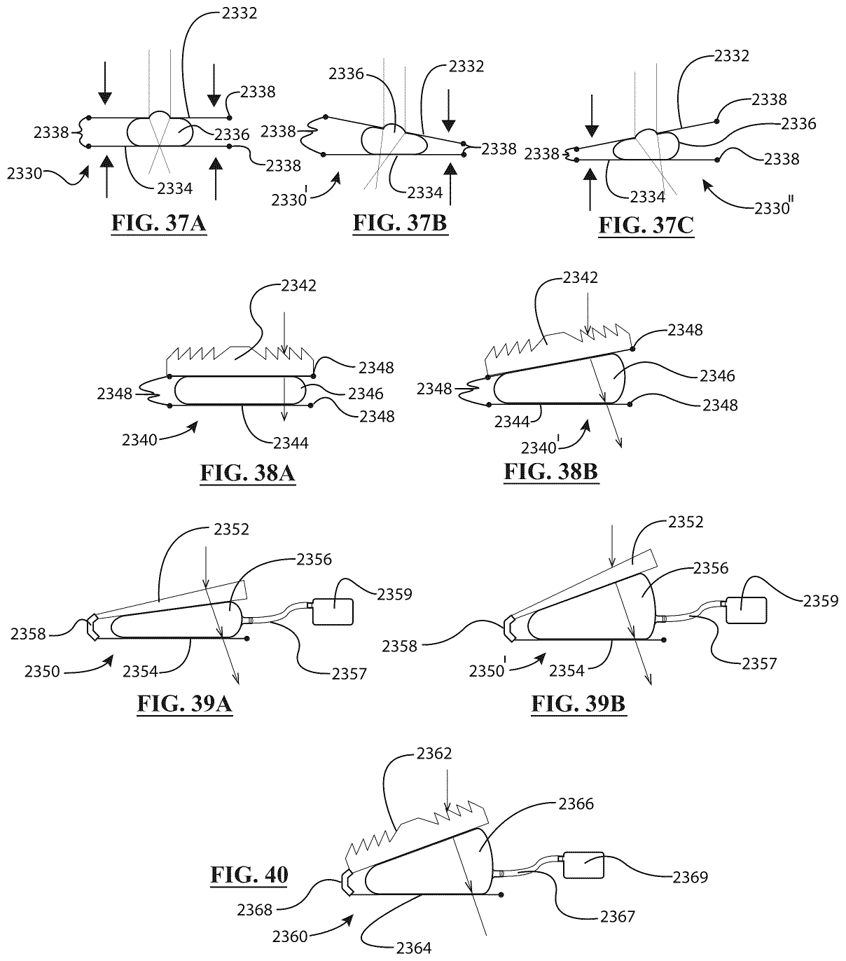

In one embodiment, the front surface of the transparent plate has an opening through which the balloon or ball can bulge out (see e.g., FIGS. 37A-37C).

In FIGS. 37A-37C, an illustrative embodiment of a tunable prism 2330 having a first movable transparent plate 2332 with a central opening and a second stationary transparent plate 2334 is depicted. In the embodiment of FIGS. 37A-37C, the first movable transparent plate 2332 is separated from the second stationary transparent plate 2334 by a transparent flexible polymeric ball 2336, and the first movable transparent plate 2332 has a central opening through which the ball 2336 can bulge out (see FIGS. 37A-37C). The transparent flexible polymeric ball 2336 may be made of silicone or another transparent elastic polymer. The first movable transparent plate 2332 and the second stationary transparent plate 2334 may be made from a transparent glass or plastic material. In the embodiment of FIGS. 37A-37C, the plates 2332, 2334 are provided with selectively activatable electromagnets 2338. In FIG. 37A, the electromagnets 2338 are not activated, and the plates 2332, 2334 are disposed parallel to one another. In FIG. 37B, the electromagnets 2338 on a first side are activated such the plates 2332, 2334 of the tunable prism 2330′ are disposed in a first vision correction configuration. In FIG. 37C, the electromagnets 2338 on a second side are activated such the plates 2332, 2334 of the tunable prism 2330″ are disposed in a second vision correction configuration.

In FIGS. 38A and 38B, an illustrative embodiment of a tunable prism 2340 having a first movable transparent plate 2342 with a diffractive upper surface and a second stationary transparent plate 2344 is depicted. In the embodiment of FIGS. 38A and 38B, the first movable transparent diffractive plate 2342 is separated from the second stationary transparent plate 2344 by a transparent flexible polymeric ball or balloon 2346. The transparent flexible polymeric ball or balloon 2346 may be made of silicone or another transparent elastic polymer. The first movable transparent diffractive plate 2342 and the second stationary transparent plate 2344 may be made from a transparent glass or plastic material. In the embodiment of FIGS. 38A and 38B, the plates 2342, 2344 are provided with selectively activatable electromagnets 2348. In FIG. 38A, the electromagnets 2348 are not activated, and the plates 2342, 2344 are disposed parallel to one another. In FIG. 38B, the electromagnets 2348 on one side are activated such the plates 2342, 2344 of the tunable prism 2340′ are disposed in a vision correction configuration.

In one embodiment, the front transparent plate is made from a prism that can be adjusted by increasing or decreasing the fluid in it, from zero to 20 prismatic diopter or more, by a pump controlled by the software of the system.

In one embodiment, the surface of the transparent plate has Fresnel or diffractive grooves influencing the direction of the light passing through it, the position of the plate can be moved by increasing or decreasing the fluid in the transparent bag located between the two plates, and the plates are joined by a hinge on one side of the plates to increase or decrease the prismatic effect (see e.g., FIGS. 39A, 39B, and 40) where the front transparent plate is a prism moving up or down by the pressure of the transparent fluidic bag via a pump controlled by the software.

In FIGS. 39A and 39B, an illustrative embodiment of a tunable prism 2350 with a first movable transparent plate 2352 and a second stationary transparent plate 2354 is depicted. In the embodiment of FIGS. 39A and 39B, the first movable transparent plate 2352 is separated from the second stationary transparent plate 2354 by a transparent balloon 2356. The transparent balloon 2356 may be made of silicone or another transparent elastic polymer, and can be filled with a fluid (e.g., water or other transparent liquid) via a tube 2357 that can be connected to a pump 2359. In the embodiment of FIGS. 39A and 39B, the plates 2352, 2354 are connected to one another by a joint or hinge 2358. The spacing between the plates 2352, 2354 is dependent upon the degree to which the balloon 2356 is inflated. In FIG. 39A, the tunable prism 2350 is in a first vision correction configuration, where the plates 2352, 2354 have a first spacing between them. In FIG. 39B, the tunable prism 2350′ is in a second vision correction configuration, where the plates 2352, 2354 have a second spacing between them that is larger than the first spacing of FIG. 39A (i.e., the balloon 2356 is more inflated in FIG. 39B than in FIG. 39A).

In one embodiment (e.g., refer to FIG. 40), the front plate is made from a Fresnel prism-like or diffractive grating, thereby influencing the direction of incoming light and its position is controlled by the transparent fluidic bag, the pump, and the units software.

In FIG. 40, an illustrative embodiment of a tunable prism 2360 having a first movable transparent plate 2362 with a diffractive upper surface and a second stationary transparent plate 2364 is depicted. In the embodiment of FIG. 40, the first movable transparent diffractive plate 2362 is separated from the second stationary transparent plate 2364 by a transparent balloon 2366. The transparent balloon 2366 may be made of silicone or another transparent elastic polymer, and can be filled with a fluid (e.g., water or other transparent liquid) via a tube 2367 that can be connected to a pump 2369. In the embodiment of FIG. 40, the plates 2362, 2364 are connected to one another by a joint or hinge 2368. The spacing between the plates 2362, 2364 is dependent upon the degree to which the balloon 2366 is inflated. In FIG. 40, the tunable prism 2360 is in a vision correction configuration, where the first movable transparent diffractive plate 2362 is diagonally oriented relative to the second stationary transparent plate 2364.

In one embodiment, the tunable lenses can be used for various purposes in medicine, a camera, a car, automated factories, remote-controlled drones, airplane, missiles, telescopes, security systems, or for use by children or adults with strabismus.

In one embodiment, the surface of the front plate of the tunable prism can be smooth, diffractive, or a finer meta optic with fine grooves.

In one embodiment, the fluidic ball or balloon provides the up and down motion of a front transparent prismatic plate that replaces a flat surface and is connected via a hinge to the back plate. The front plate moves by the pressure applied to it via the fluid-filled ball or balloon by a pump that can be activated one time only and stop, or thousands of times or more, for scanning an image or the field of view controlled by software while the pump injects fluid in the transparent ball or balloon and creates oscillations and changes in the direction of the field of view of an attached camera and/or corrects the ocular prismatic deviation.

In one embodiment, a tunable prism that includes a first transparent plate and a second transparent plate. The first transparent plate is separated from the second transparent plate by a transparent gel, or by a transparent bag filled with the transparent gel; and a tilt of at least one of the first and second transparent plates is configured to be modified so as to adjust a prism diopter of the tunable prism.

In one embodiment, the digital images obtained by the system, which includes the tunable prism and the light field camera or an OCT, can be recreated in 3D fashion using Metaverse's software and headset for the patient and his or her doctor so that they can viewed it in 3D format using a headset or goggles, a computer, or smart phone with AR or VR (e.g., by Amazon, Microsoft or Facebook) as a kiosk or moveable format for remote patient's evaluation and communication to image at the retina, lens, or cornea etc. from any direction etc.

In one embodiment, the images obtained by the system can be recreated in 3D fashion manner using Metaverse's software and a AR or VR headset combined with dynamic facial recognition for the patient and his or her doctor so that the image can be viewed in 3D format by both the doctor and the patient, assisting the doctors in presenting the pathology to the patient, or presenting the pathology in a different color to distinguish it from the normal structures of the patient.

In one embodiment, the images obtained by the system can be recreated in a 3D manner using Metaverse's software and a AR or VR headset combined with dynamic facial recognition and combined with retinal vessels or optic nerve vessels to add to the person's recognition to include for teaching or exclude other people to weed out intruders, hackers, etc. and to keep the patients privacy secret and inaccessible to others all the time.

In one embodiment, the phoropter is used for children where the size of the headset is for an IPD of 40-50 mm in children ages 1-5.

In one embodiment, the ocular part of the ocular of the instrument requires an IR camera for photography of the position of both eyes having strabismus or not to measure the inter-pupillary distance of a child (e.g., via a software).

In one embodiment, in children, the use of a phoropter or camera requires a change in the visual display from static to dynamic video and an attractive sound (e.g., cat or bird, etc.,) and colored animation for the child above 1 year to get attracted to it.

In one embodiment, in the use of a phoropter or camera in children, the front part of ocular site should accommodate the head of a child and an adult, to position the child's head and align the eyes with mostly flat oculars for both eyes looking at the animation.

In one embodiment, in children ages 1-5 years, it is important to find if there are differences in refractive power between both eyes to prevent strabismus and amblyopia (gradual loss of the ability to see if left uncorrected) using the unit, and then provide the refractive errors of both eyes, images of the eyes and retina, etc. and prescription glasses, or tunable prisms to correct the refractive error, or in some cases, the child may be referred to the doctor for a strabismus surgery.

In one embodiment, the phoropter alone or in combination with a camera as described in the Applicant's tunable fluidic camera patent (e.g., U.S. Pat. No. 10,606,066) so that the various components of the eye such as the cornea, lens, and the retina can be evaluated in 2-D or 3-D manner using attachment of optical coherent tomography or any other camera to the phoropter so that a beam of light passes initially through the phoropter and the optical aberration of the eye is corrected for uses as an optical home monitoring device for evaluation of a patient's eye after a surgery or medical treatment. At present, home monitoring and remote communication is acceptable in assessing various diseases, such as measuring one's temperature during an infection or measuring the blood pressure in hypertensive individuals, heart rhythm, in arrhythmia, or blood glucose in diabetics, etc. In ophthalmology, measuring the intraocular pressure (IOP) regularly is important because an increase of the IOP or its fluctuation stresses the retinal ganglion cells and their axons leading to loss of retinal cells, and gradual loss of visual field, thus eventually causing blindness. One of the hallmarks of the home monitoring devices is the simplicity of their use and the accuracy of the information they provide so that even an uneducated person understands it, and the patient can communicate the information if it is not normal to her or his health care professional. Often the information is in a form of a numerical value, indicating what is considered normal (e.g., the 37° C. degree body temperature or the 12-20 mm Hg range of the normal IOP, or the normal blood glucose range of below 100 mg/dL). In ophthalmology, ophthalmic home care devices, such as home tonometer are used by the patient or another trusted person to assess the IOP of a patient under treatment at home regularly. This enhances self-reliance of the patient to adhere to a certain regimen or taking the prescribed medication, and communicating the results to the professional remotely without the need to drive to the doctor's office. It also reduces the patient's concern about her or his disease after a doctor's visit or after a surgery, and allows the patient to observe an improvement or stabilization of the disease after treatment.

In one embodiment, an automated phoropter and refractometer is made in a single unit eliminating all the phoropter lenses, replacing them with three fluidic lenses, under the control of Shack-Hartmann sensor and associated software that modifies the refractive power of the lenses by injecting or removing fluid from the lenses automatically. The unit corrects the refractive aberration of the eyes (spherical and cylindrical) without changing or replacing the lenses within 10 seconds, it keeps the visual display in the view of the patient so that the patient observes actual improvement of the image without losing the image (i.e. the visual display does not disappear in this process of increasing or decreasing the refractive power of the fluidic lenses as is the case with the standard phoropters, once the patient looks at an illuminated target and the visual display inside the system through the goggles, the unit automatically with its LED, Shack-Hartmann sensor and its software, modifies the refractive power of the units' spherical and cylindrical fluidic lenses, for far and near using its fluidic pump, correcting the aberration of the eye to bring the visual display in focus for the patient's retina. The visual display used for children has animated images with sound to attract their attention, there is no need to ask the patient “one or two, which is better?” via a nurse or a doctor while changing the lenses in front of the eye of the patient which is the basis of the present phoropters with the subjective refraction.

In one embodiment, the unit produces a prescription that corresponds to the amount of correction needed for the patient to see 20/20 or 20/25 seeing, e.g., a Snellen chart or another equivalent image display, eliminate guessing by the patient for the correction of refractive power. Once corrected, if the patient does not have a 20/20-20/25 vision, it should be considered an abnormal visual acuity caused by a disease process affecting the cornea, the lens or the retina and shall require evaluation by a healthcare professional.

In one embodiment, the unit also can present a different visual display that does not require a understanding of the English language, such as using “E” letter or images of animals. Because the entire system does not require “back and forth” oral communication, the patients can cither perform the test at home or, if needed, with the assistance of a trusted person and the results of the reading the lines of the chart from 20/0 to 20/400 and the refractive power with their prescription are communicated to the patient's ophthalmologist or optometrist remotely. Any variation of the results from the normal value or a previously obtained value by the ophthalmologist or optometrist is reported to the patient's doctor.

In one embodiment, the phoropter system not only simplifies the screening of a large segment of the population including elderly, school children, etc., but also similarly, a huge number of diabetic patients (30 million in the U.S. alone) that unfortunately can develop diabetic retinopathy or patients with age-related macular degeneration (20 million in the U.S.), etc. can develop changes in the retina or the choroid which can be detected by the camera or the OCT with AR/VR software, if their visual acuity has been affected, to be treated by intravitreal injection of medication, etc. and followed at home and their vision can be evaluated at home repeatedly with ease to be checked for stabilization, or improvement, or worsening of the condition after treatment using the Metaverse or AI and AR software that otherwise would require an office visit.

In one embodiment, the home monitoring phoropter, camera or OCT device simplifies the communication between the doctors and the patients, and may prevent loss of sight in patients that can be treated. Like the other home monitoring devices, this device creates an insight into the visual system and its function of the cornea, lens and the retina, etc. making the patients in charge of their own vision.

In one embodiment, a home monitoring system is used for evaluating a refractive error and/or an ocular disease of a patient, wherein the home monitoring system includes a fluidic phoropter, a camera to photograph the retina, AR/VR software, and a Shack Hartmann sensor is used for evaluation the patients with a disease process before and after treatment in a daily or weekly or monthly fashion and the results of the visual acuity is communicated to the doctor through the internet.

In one embodiment, the modified AR or VR with AI software, a tunable camera, and tunable prism (e.g., refer to U.S. Pat. No. 11,372,230, the disclosure of which is incorporated by reference herein in its entirety) are used for home diagnosis of an eye disease and bot-assisted artificial intelligence (AI) is used to ask questions and/or respond to the patient's questions to shorten the exam time by limiting the areas of interest for measuring and refinement of visual acuity and follow up of the eye diseases and recognition of the ocular pathology and their changes over a time period involving the cornea, lens, vitreous gel, retina and its vasculature, and optic nerve head, and communicating with the patient and the doctor, etc.

In one embodiment, the bot-assisted AI asks questions or responds to the patient's questions to limit the potential of eye diseases involved, and thereby shorten the exam time of the patient.

In one embodiment, augmented intelligence AR or VR with a phoropter and a camera (see U.S. Pat. Nos. 9,016,860 and 9,191,568) are used for diagnosis of ocular diseases or as a home monitoring device in diabetic patients with diabetic retinopathy or diabetic macular edema, age-related macular degeneration, retinal vascular diseases, by using the collimated light that enters the eye through a prismatic lens in front of the eye to reach the retina where the reflected light from the retina, vitreous and lens and cornea passes through a dichroic mirror which diverts the light from the eye to a camera that records the images of the retina, vitreous, lens, cornca, and the images are analyzed with augmented intelligence or bot-assisted artificial intelligence (AI) software to rapidly diagnose a disease or its stage in a diseased cornea, lens, vitreous, or retina and optic nerve, then the analyzed images are transmitted via the internet to the patient and his or her ophthalmologist or optometrist along with the refractive errors corrected from the tunable lenses and corrected values obtained by the tunable prisms' software for bilateral vision. In one embodiment, the fluidic camera or the phoropter (see U.S. Pat. No. 9,191,568) is equipped with dynamic facial recognition software and optical coherence tomography (OCT) and bot-assisted artificial intelligence (AI) software used for home monitoring by imaging where the cornea, lens, vitreous, and retinal images of the patient are scanned rapidly with the fluidic lens camera and its dynamic imaging and AI software or a neuromorphic camera records rapidly the dynamic changes of a structure(s) and analyzes them with AI software and the information is immediately transmitted to a doctor to confirm the diagnosis of a disease, such as diabetic macular edema, degree of the sub-retinal fluid, or the existence or the progression of an age-related macular degeneration or a central vein occlusion, or branch vein or artery occlusion, or retinitis pigmentosa, or presence or absence of a tumor or optic nerve head edema, or changes due to glaucoma or the retina in diabetic retinopathy or change in the peripapillary micro-vasculatures, the retinal thickness, or cellular changes in the retina or choroid, etc.

In one embodiment, with reference to FIG. 31, the fluidic phoropter and a camera (see U.S. Pat. Nos. 9,016,860 and 9,191,568) can automatically focus the beam on the patient's retina to check the visual acuity and photograph the retina, and the Shack Hartmann sensor assembly 2270 of the unit 2244 can be connected to the basic unit or it can be connected to the unit via the internet, thereby making the unit portable and useable as a home monitoring system to follow a patient or evaluate a new patient for his or her refractive error or an ocular disease, as the illustration in FIG. 31 depicts, while the patient is observing a visual display, an infrared beam enters an activated rapidly oscillating tunable prism and dichroic mirror 2258 in front of the pupil of the eye 2260, scanning the cornea, lens, retina of the eye 2260, and returns back passing through the fluidic lenses 2256 and is diverted via a prismatic beam splitter (PBS) 2254 and via a relay lens 2252 toward another prismatic beam splitter 2250 either toward a Shack-Hartmann sensor 2248 or a camera 2246, here the activated sensor's software can directly correct the fluidic lenses 2256 to correct the optical aberration of the eye or a sensor can send the signal through the cloud to remotely located devices (e.g., prismatic beam splitter 2268 and Shack Hartmann sensor assembly 2270) or locally located device(s) inside the unit; a sensor can activate fluidic lenses 2256 via the cloud and AI remotely in order to activate the pumps to modify the fluidic lenses' shape which corrects the refractive errors of the eye while seeing the visual display (e.g., with a fixation chart or an object). In the illustrative embodiment of FIG. 31, infrared beam may be generated by a light-emitting diode (LED) emitter 2266 and transmitted by means of a fiber 2264, and then the infrared beam may be diverted by a concave or elliptical mirror 2262 before entering the tunable prism and dichroic mirror 2258 of the unit 2244.

In one embodiment, similarly, the light that is diverted to the oscillating tunable prism and fluidic camera can directly transmit the scanned image information to the software of its digital camera or as above the in-focus of scanned images (signals) of the person's retina, lens, and cornea which can be sent to the cloud or elsewhere to be presented and the AI software recognizes the patient or the structure if the patient has been photographed and the images of the scanned cornea, lens, and the retina, etc. are analyzed with bot-assisted visual acuity measurement and AI software to recognize the changes in visual acuity or recognize the patient, and a disease process to extract the diagnostic information/images, such as improvement or worsening of a condition which are transmitted via the internet to the patient or his or her doctor.

In one embodiment, the home monitoring system can provide the information of the optical aberration of the eye simultaneously with images of the cornea, lens, retina, etc. to be analyzed with bot-assisted AI software for the presence or absence of a disease process, such as retinitis pigmentosa, age-related macular degeneration, diabetic retinopathy, macular edema, etc.

In one embodiment, with reference to the system 2272 of FIG. 32, the light enters the eye 2274 and exits after passing through the fluidic lenses 2280 to a sensor 2286 that sends the information through the cloud to a Shack-Hartmann system 2288 and with bot-assisted AI software located elsewhere, after analyzing the information with AI, the signals are sent back via the cloud to the first unit located in the initial place with the unit to activate the pump of the fluidic lenses 2280 and correct the refractive error of a patient while he or she is looking at a visual display (e.g., fixation target 2282) in the first location, then the tunable fluidic camera (e.g., as described in the Applicant's U.S. Pat. No. 10,606,066) obtains images from the retina through its activated oscillating or scanning tunable prism, and the images and diagnosis are transmitted via cloud computing to the patient's smartphone and his doctor as is done with any smartphone camera, thereby simplifying home monitoring of the disease process of the cornea lens and the retina. Also, as shown in the illustrative embodiment of FIG. 32, similar to that described above for the system 2244 of FIG. 31, the system 2272 may further include a concave or elliptical mirror 2278 and a prismatic beam splitter (PBS) 2276 that transmit the light from the light source to the eye 2274 of the patient. In addition, as shown in the illustrative embodiment of FIG. 32, similar to the system 2244 of FIG. 31, the system 2272 may further include a relay lens 2284 between the prismatic beam splitter (PBS) 2282 and the sensor 2286.

In another embodiment, FIG. 33 shows that because of the size, thickness and weight (see U.S. Pat. No. 11,372,230) of the tunable prisms, fluidic lenses and the imaging camera, the Shack-Hartmann sensors, the phoropter, and a bot, the standard goggles glasses are converted to a larger version that made the goggles too bulky and heavy to carry them on the nose. Therefore, the enlarged, modified AR/VR system are placed in front of the eye on a small portable table or a small AR or VR kiosk model, or for use as home monitoring device where the unit is kept, with the tunable prism, fluidic lenses, the bot, the camera and a small sized computer or a chip. However, the Shack-Hartman sensor with or without fluidic lenses and electronics and bot-assisted AI, AR, VR software and with dynamic facial recognition software (e.g., as described in the Applicant's U.S. Pat. No. 11,309,081) are either positioned locally or reached with cloud computing with two-way communication back to the unit, where this binocular system evaluates simultaneously the visual acuity, stereovision, using an OCT for imaging the cornea, lens, vitreous, and retinal pathology in various ophthalmic and systemic diseases, including the function of oculomotor system affecting convergence and accommodation, etc.

In FIG. 33, an illustrative embodiment of a pair of augmented reality (AR) or virtual reality (VR) goggles are diagrammatically depicted. In FIG. 33, it can be seen that the AR or VR goggles include tunable prisms 2290, 2292, fluidic lenses 2294, 2296, and electronics that are connected via the cloud to a remote Shack Hartmann sensor with artificial intelligence (AI) software for binocular vision, imaging of the cornea, and/or diagnosis of a disease of eyes 2298, 2299 of a wearer of the AR or VR goggles.

In one embodiment, all fluidic lenses and the Shack-Hartmann system can be replaced with a light field camera or preferably a Peyman light field camera (see e.g., U.S. Pat. No. 10,606,066) with its software and algorithm with or without a bot and present fast in-focus images of an external object such as cornea, lens, vitreous, and retina at any point and differentiate the normal structure from the diseased structure with its computer and AI software and recall virtual 2-D or 3-D images at any point in the cornea, lens, vitreous, and retina.

In one embodiment, all fluidic lenses and the Shack-Hartmann system can be replaced with a light field camera or preferably Peyman light field camera (see e.g., U.S. Pat. No. 10,606,066) with its software and algorithm with or without a bot and present fast in-focus images of an external object, such as a cornea, lens, vitreous, and retina at any point and differentiate the normal structure from a diseased structure with its computer and AI software and recall virtual 2-D or 3-D images at any point in the cornea, lens, vitreous, and retina where the system is combined with a slightly larger modified AR/VR goggles for in-home diagnostics, or communicating with its smartphone computer to the patient or his or her doctor via internet.

In one embodiment, the smartphone can be combined with a small Peyman light field camera or optical coherence tomography (OCT) replacing the presently available camera that produce only 2-D in-focused images, thereby producing 3-D images of any object in its field of view and when combined with dynamic facial recognition, one can recognize any object or moving object in its field.

In one embodiment, the light field camera or Peyman light field camera can be equipped with an infrared LED or laser for night vision photography in the dark providing sharp infrared (IR) images of an object, human, or animal, or potentially from structures inside the body's cavity such as eye, cornea, lens and retina, etc., that can be reached with an IR beam with without a flexible fiberscope.

In one embodiment, the smartphone can be combined with a small Peyman light field camera (see e.g., U.S. Pat. No. 10,606,066) with an oscillating tunable prism for light or infrared (IR) wide angle imaging and scanning a wide field of view with its use for security systems and the military, or in medicine, physical activity, etc. and the obtained images or video can be analyzed with AI software, transmitted via cloud computing to any desired place, or can be encrypted prior to sending the information (e.g., images) out. This system also can be combined with a bot for recording, sound or words information or two-way communication etc. combined with or without dynamic face recognition, etc. (e.g., as described in the Applicant's U.S. Pat. No. 11,309,081).

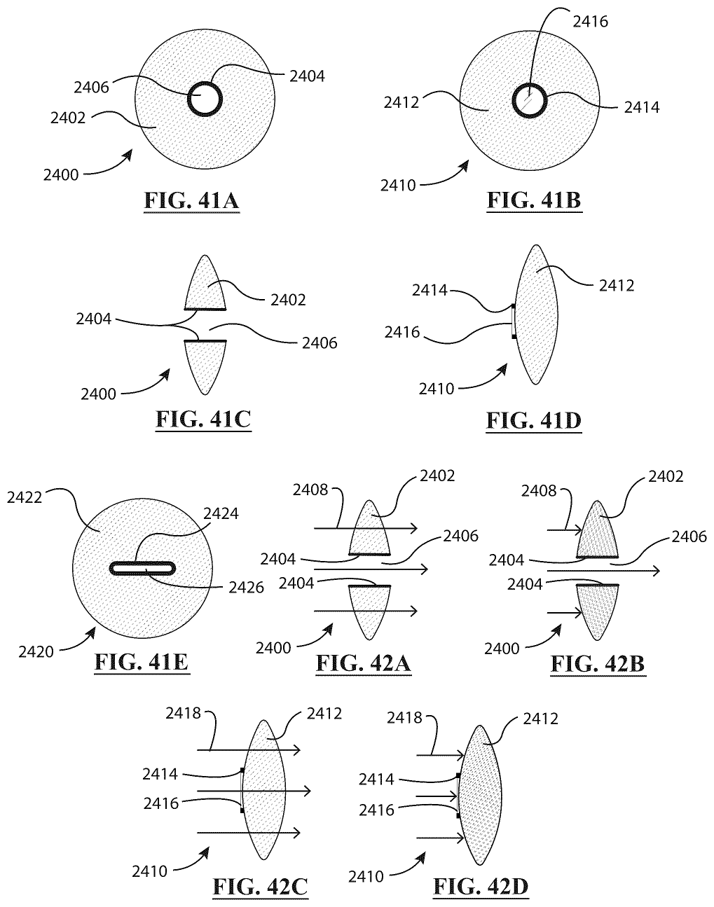

Now, with reference to FIGS. 41A-50C, corrective lenses (e.g., transitional lenses with permanently darkened pinholes) for correction of refractive errors and treatment of photophobia in children and albinos will be described. Also, these transitional lenses with permanently darkened pinholes may be used for virtual reality or augmented reality goggles to focus the beam automatically on the retina and simultaneously modify the glare or discomfort, particularly in albinos or people with very light skin or young children.

In one embodiment, the refractive error of very young children and albinos is corrected with a modification of the refractive power of their eye(s) by prescribing glasses or contact lenses that correct the refractive errors of the eye. The corrective glasses or contact lens may additionally include a central pinhole (e.g., through hole) with a darkened wall which compensates simultaneously for myopia, farsightedness, or astigmatism; the light passes through the pinhole directly to the retina without being refracted, and gives the albinos or children the possibility of fine focus for far and near with both eyes, thereby preventing development of amblyopia or lazy eye. This ability of seeing objects in focus stabilizes the normal growth of the eye contributing to lowering the incidence of high myopia with its subsequent negative effect on the vision.

In one embodiment, the glasses, contact lenses, the intraocular lenses, or the corneal inlay are made with a central pinhole (e.g., through hole) with a darkened wall. The central pinhole may encompass the central 1-5 mm diameter of the lens depending on the location of the pinhole lens. For example, when the lens is inside the eye or in the lens capsule after cataract surgery, the pinhole may be 1-2.5 mm in diameter, or when the lens is inside the cornea, the pinhole may be 1-3 mm in diameter. As another example, when the lens is over the cornea as a contact lens, the pinhole may be 1-4 mm in diameter, or when the lens is outside the eye as glasses, the pinhole may be 1-4.5 mm in diameter depending on the size of the pupil of the patient.

In one embodiment, the glasses, contact lenses, the intraocular lenses, or a corneal inlay are made with a virtual pinhole with a circular or ring-shaped mask with central 1-2 mm circular hole, or the central hole can be a rectangular slit, an oval-shaped silt, or oval-shaped mask. The slit can be oriented in a horizontal or vertical direction (e.g., with a length of 1-3 mm). The opening of the slit can have a width of 1-2 mm in a direction 90 degrees opposite to its length depending on the location of the inlay inside or outside the eye (e.g., in the lens capsule after cataract surgery, inside the cornea, or over the cornea as a contact lens). When the lens is inside the cornea, the pinhole may have a diameter of 1-3 mm. When the lens is over the cornea as a contact lens, the pinhole may have a diameter or length of 1-4 mm and a width of 1-2 mm. When the lens is outside the eye as glasses, the pinhole may have a diameter or length of 1-5.5 mm and a width of 1-2 mm in the opposite direction, or depending on the size of the pupil, between 1-3 mm.

In one embodiment, the lens or intraocular lens (IOL) is made to respond to the light by blocking most, but not all, of the light reaching the retina, while the central lens areas where the pinhole/slit is located permits the light to pass through to reach the central retinal area regardless of the dioptric power the lens (e.g., refer to FIGS. 41A-41E).

FIG. 41A shows a transitional lens 2400 containing chromophores and a pinhole 2406 passing through the lens body 2402 that is surrounded by a darkened wall 2404.

Advantageously, the central pinhole 2406 of the transitional lens 2400 with the darkened wall 2404 prevents glare during the day and simultaneously focuses the light on the retina for near to far vision through the pinhole 2406 for any distance in front of the eye.

FIG. 41B shows a transitional lens 2410 with a lens body 2412 containing chromophores and a mask 2414. The mask 2414 defines a virtual pinhole 2416 with no aperture passing through the lens body 2412. In FIG. 41B, the mask 2414 occupies a larger part of the lens surface so as to reduce night vision for the patient.

FIG. 41C is a side cross-sectional view through the lens 2400 of FIG. 41A, which shows the permanently darkened wall 2404 of the pinhole 2406. It is important to note that a pinhole 2406 without a darkened wall 2404 would create glare that would make it difficult for the wearer of the lens to see.

FIG. 41D is a side cross-sectional view through the transitional lens 2410 of FIG. 41B, where the lens body 2412 is provided with the chromophores and a mask 2414 that can be in the front or back surface of the lens with the disadvantage of diminishing light reaching the retina at night.

FIG. 41E shows a transitional lens 2420 with a lens body 2422 containing chromophores and a central slit 2426 passing through the lens body 2422 that is surrounded by a darkened wall 2424.

In one embodiment, the pinhole with the darkened wall is in a form of a circular aperture that passes through the lens body of a pair of glasses. Alternatively, the pinhole or a virtual pinhole having a mask may be circular, oval-shaped, or slit-shaped, while the rest of the glasses may be in a form of corrective refractive or diffractive lens, or progressive lens which can correct for refractive deficiencies for both near and far.

In one embodiment, the transitional lens is a contact lens, an intraocular lens, or an inlay inside the cornea. The pinhole in the transitional lens can be elongated or a slit toward the astigmatic axis of the eye to eliminate or reduce the astigmatism.

In one embodiment, the slit in the lens body of the transitional lens is shaped like a dog bone with a circular or dumbbell-shaped extension at both ends of the slit to prevent light scattering.

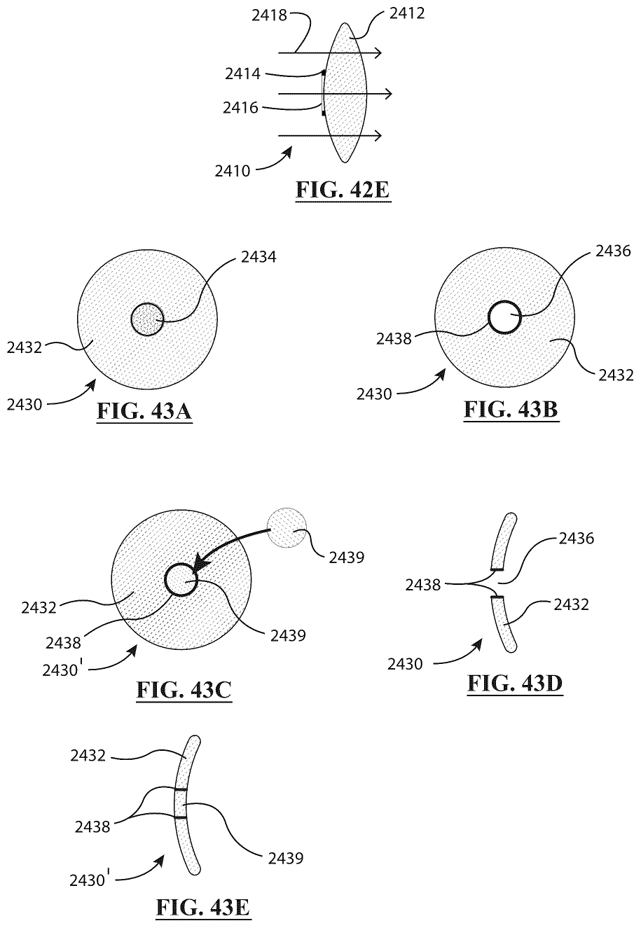

FIGS. 42A and 42B show the cross-sectional of the FIG. 41A lens 2400 with activated chromophores with light (denoted by arrows 2408) permitted to pass through the pinhole 2406. Alternatively, the pinhole 2406 could be in a form of a virtual pinhole (mask) that prevents light from reflecting off its surface, while the chromophores block most of the light to passing through the remainder of the lens body when the wearer is exposed to a high lighting level (e.g., during the daytime). In the low level lighting condition of FIG. 42A (e.g., at night), the chromophores in the lens body 2402 are not activated such that light 2408 passes through the lens body 2402 outside of the pinhole 2406. In the high level lighting condition of FIG. 42B (e.g., during the daytime), the chromophores in the lens body 2402 are activated such that light 2408 is prevented from passing through the lens body 2402 outside of the pinhole 2406.

FIGS. 42C and 42D show the cross-sectional view of a transitional lens 2410 with a mask 2414 that blocks the light 2418 from passing through the lens body 2412. However, some limited light 2418 can pass through the lens by choosing a selective chromophore (e.g., as shown in FIG. 42E).

In one embodiment, the wall of the pinhole is darkened by various means such as black paint, or a dark cylindrical tube, or a dumbbell-shaped element that is placed inside the central hole to secure it inside the pinhole (see e.g., FIGS. 41A and 42A).

FIGS. 43A-43C shows a technique to create a combination of two transitional areas in one lens 2430 where the central circular part 2436 is cut away (in FIG. 43B) using a laser, etc., and then the central area is replaced with another lens or flat clear polymer or glass with a darkened wall (e.g., having only a UV or other light wavelength of the light absorber). That is, the central area of the lens body is replaced with a central lens section 2439 (see FIGS. 43C and 43E) having a different light absorber material (e.g., a light absorber material sensitive only to ultraviolet (UV) light).

FIG. 43A shows a transitional lens 2430 with a lens body 2432 containing chromophores and a central circular dark portion 2434, which is cut away (as depicted in FIG. 43B), thereby leaving a circular aperture 2436 bounded by a darkened wall 2438. FIG. 43D is a cross-sectional side view of FIG. 43B.

FIGS. 43C and 43E show the front view and side view of a lens 2430′ having a combination of two transitional areas, where the central lens section 2439 (e.g., UV absorbent section) is, for example, glued in place, while still maintaining the pinhole effect function of the lens, or a mask is placed in the front or back surface of the lens 2430′ that can produce the pinhole effect.

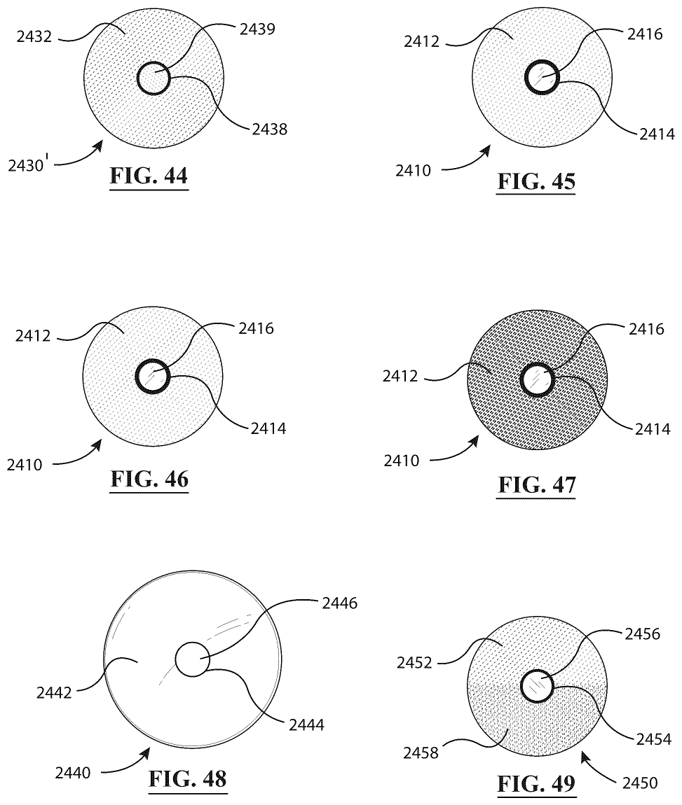

FIG. 44 is a front view of a transitional lens 2430′ that is similar to the transitional lens of FIG. 43C, where the peripheral portion of the lens body 2432 contains the chromophores for absorbing all wavelengths of light, while the central lens section 2439 is formed from a different light absorber material that is sensitive to only ultraviolet (UV) light.

FIG. 45 is a front view of a transitional lens 2410 that is similar to the transitional lens of FIG. 41B, where the lens body 2412 comprises a ring-shaped mask 2414 defining a virtual central pinhole 2416 with no transitional properties and a peripheral lens body portion 2412 surrounding the mask 2414 that contains chromophores for operating as a transitional lens.

FIG. 46 is another front view of the transitional lens 2410 of FIG. 45, wherein the transitional lens 2410 is depicted in a low level lighting condition where the chromophores in the peripheral lens body portion 2412 are not activated such that light passes through the lens body 2412 outside of the pinhole 2416.

FIG. 47 is yet another front view of the transitional lens of FIG. 45, wherein the transitional lens 2410 is depicted in a high level lighting condition where the chromophores in the peripheral lens body portion 2412 are activated such that light is prevented from passing through the lens body 2412 outside of the pinhole 2416.

FIG. 48 is a front view of a combined transitional and presbyopia lens 2440 in a form of a contact lens, according to another illustrative embodiment of the invention, where the combined transitional and presbyopia lens 2440 is provided with a lens body 2442 and a circular through hole or virtual pinhole 2446 bounded by a darkened wall 2444.

FIG. 49 is a front view of a combined progressive and transitional lens 2450, according to yet another illustrative embodiment of the invention, where the lens body 2452 comprises a central pinhole 2456 with no transitional properties, which is bounded by a mask 2454, and a peripheral lens body portion 2458 surrounding the mask 2454 that contains chromophores for operating as a transitional lens.

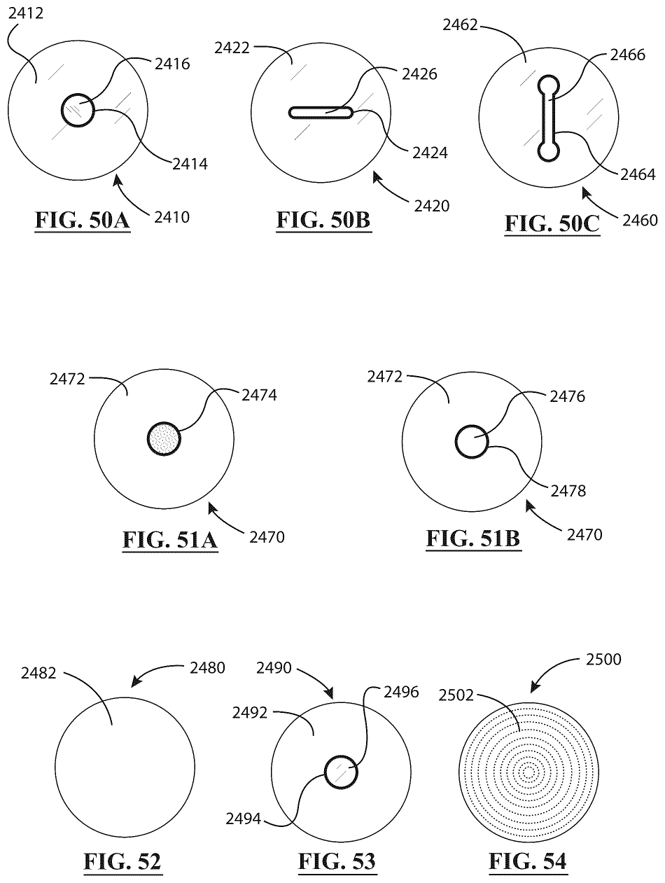

FIG. 50A is a front view of the transitional lens 2410 with a lens body 2412 and a circular virtual pinhole 2416 bounded by the dark mask 2414.

FIG. 50B is a front view of the transitional lens 2420 with the central horizontal slit 2426 passing through the lens body 2422 that is surrounded by a darkened wall 2424.

FIG. 50C is a front view of another transitional lens 2460 with a central vertical slit 2466 passing through the lens body 2462 that is surrounded by a darkened wall 2464, according to yet another illustrative embodiment of the invention, wherein the central vertical slit 2466 may be provided with dumbbell-shaped extensions at both ends of the slit 2466 to prevent light scattering.

In one embodiment, the front mask is slightly larger in diameter than the rear mask so as to reduce glare, but permit maximum light to reach the retina at dawn or dusk.

In one embodiment, the central lens or the peripheral lens forming the glasses are either 3-D printed or molded, each piece separately, and the chromophores are added selectively to a part or to the one or both surfaces of the lens or areas independently or mixed with the polymer, or the lenses are subsequently encapsulated in a non-permeable thin layer of carbon by electron spattering, plasma spattering, nanotechnology, or other known methods, or other transparent molecules to retain the chromophore are permanently inside the intraocular lens (IOL) to prevent the exit of the chromophore even in a fluidic medium.

In one embodiment, the lens is formed from silicone or polyurethane, silicone, glass, polycarbonate, plastic, SR-91, CR-39, colorized plastic, or a hydrophobic acrylate material or a hydrophilic lens material, or a cross-linked polyisobutylene (xPIB), polycarbonate or other transparent polymers, foldable or hard combined with chromophores and easily molded, organic, or non-organic or a mixture of acrylic and organic polymers, such as collagen etc. with or without an antireflective coating on their surfaces and with or without a polarized filter.

In one embodiment, the lens surface is made of gaseous molecules from essentially compounds selected from the group consisting of hydrocarbons, halogenated hydrocarbons, halogenated hydrocarbons and hydrogen, hydrocarbons and a halogen, and a mixture of any two or more of these compounds. The surface of the fully formed lens is highly hydrophilic which is accomplished during the polymerization process or in an additional step comprising glow discharge in the presence of oxygen or argon building an optically clear, impermeable barrier coating.

In one embodiment, the lens body is made of acrylic, methacrylate, silicone, polyurethane or hydrogel, glass or polycarbonate, crosslinked collagen and/or any transparent material or combinations thereof with crosslinked collagen, chitosan, etc. or any other crosslinked organic transparent material known in the art with or without chromophores.

In one embodiment, in treatment of an albinotic eye, a transitional lens or intraocular lens (IOL) is made of acrylic, collamer, or any transparent optical polymer with a hydrophobic or hydrophilic surface having the desired refractive power to correct the refractive deficiency of the patient, and the transitional IOL has a central pinhole passing through the lens body with a darkened wall or a virtual mask that creates a dark ring around the center of the lens, which can have a diameter of 1-6 mm or larger and a clear center with a total diameter of 1-3 mm or more, with or without a UV absorber in the case of a virtual pinhole with a mask. The transitional lens further including a peripheral transparent polymer having molecules that by exposure to light darken, but are transparent at low light, or in darkened conditions, as with transitional lenses where the lens is implanted in front of the crystalline lens of the eye behind the iris, or is used a secondary pinhole or virtual pinhole transitional intraocular lens (IOL) in front of an existing IOL where the haptics are established in the sulcus of the ciliary body and the lens works like a light protecting shield with or without refractive power. The surgical technique is similar to implanting a collamer lens in the eye in front of the crystalline lens, or in the case of an existing cataract, after its extraction, a pinhole or virtual pinhole transitional intraocular lens (IOL) is placed inside or outside the lens capsule, as known in the art.

In one embodiment, the pinhole or virtual pinhole transitional intraocular lens (IOL) is for refractive correction. In one embodiment, the pinhole or virtual pinhole transitional intraocular lens (IOL) is to enhance vision. In one embodiment, the pinhole or virtual pinhole transitional intraocular lens (IOL) is for cosmetic glasses.

In one embodiment, the lens is an intraocular lens (IOL) inside the eye. In another embodiment, the pinhole or virtual pinhole transitional intraocular lens (IOL) is placed inside the cornea, or as a contact lens, or as external glasses.

In one embodiment, the lens body or its surface contains at least one or more light-activated chromophores that darken when light-activated, the chromophore is in or on the surface of the lens polymeric material to block the incoming light by darkening the lens body depending on the intensity of the light as known in the art in transitional sunglasses.

In one embodiment, the chromophore is selected from the group consisting of a compound that selectively absorbs ultraviolet light; a compound that selectively absorbs green, or yellow, or red light; a compound that selectively absorbs ultraviolet light; a compound that selectively absorbs visible light; a compound that polarizes light; and combinations thereof.

In one embodiment of intraocular lens with the darkened aperture, the chromophore acts like a filter, preventing selectively the penetration of light below blue, below green, or below the yellow wavelength, or below the red wavelength, or below the infrared light wavelength. This characteristic is specifically useful for an albino eye, since it permits visualizing the retina using a wavelength (e.g., a wavelength that permits the wavelength above yellow light to pass through it) in order to examine the retina or apply a laser whose wavelength is at red or a longer wavelength (e.g., an infrared laser for albino patients with retinal pathologies that require laser therapy, such as diabetic retinopathy) and eliminate the rest of the white light wavelength, thus reducing the glare and photophobia for the patient.

In one embodiment, the use of the chromophore that blocks blue and green would reduce the glare in these patients while permitting laser coagulation or photography, or for optical coherence tomography of the retina as needed.

In one embodiment, glare is a recognized disability (e.g., outdoors, or in eyes with minimal or no natural pigment, such as in albinos), or when the iris is damaged or lost after trauma, glare reduces visual performance and/or visual acuity because of stray light reducing retinal contrast.

To the inventor's knowledge, until now, there are no intraocular lens (IOL) or other lenses for an albinotic eye that has a pinhole with a darkened wall or a virtual pinhole with a mask for proper focusing of the light on the fovea and where the lens darkens in the light, depending on the light intensity while maintaining a free passage of light through the central pinhole simultaneously. In another embodiment, the virtual pinhole is made of a ring-shaped mask and its center lacks pigment, though it is difficult to eliminate the chromophore centrally, but the patient still benefits from the pinhole effect for creating an extended focal point for near and far vision.

In one embodiment, the chromophore is a photochromic molecule, i.e., a molecule that is activated by light and, upon activation, darkens the lens. Examples of photochromic molecules that are activated by UV light are oxazines and naphthopyrans, etc. An example of a photochromic molecule that is activated by visible light is silver chloride. In one embodiment, the photochromic molecule responds to light in the UV and visible spectrum. In one embodiment, the photochromic molecule is silver chloride. In one embodiment, multiple different chromophores are used.

In one embodiment, chromophores include, but are not limited to, those that absorb UV light, those that absorb visible light, those that polarize light, and combinations of these or the chromophore can act as a filter for a specific wavelength of the light.

In one embodiment, the pinhole with the darkened wall will maintain the central darkened wall pinhole area, which remains always transparent in its center, allowing sight even after a fast transition from light to dark, and achieving no darkening of the central area upon exposure to the brightest light.

In one embodiment, with the darkened pinhole wall, a chromophore that can be activated is used to coat the lens, where coating refers to both applying the light-activated chromophore to the surface of the lens, and/or also embedding the light-activated chromophore within the lens material.

In one embodiment, the lens with the central pinhole with the darkened wall can be monofocal, bifocal, or multifocal, or the lens can be an intraocular lens (IOL), a contact lens, a corneal inlay or external to the eye as glasses. In one embodiment, the chromophores are not limited to the lens, but the adjacent glass body or glass holders can have a chromophore to prevent side radiation from entering the eye from any side.

In one embodiment, the pinhole transitional intraocular lens (IOL) with the darkened wall can be implanted in the anterior chamber, posterior chamber, in the lens capsule, or over another IOL, or over a natural crystalline lens, etc. This IOL can correct spherical or astigmatic errors, and prevent photophobia.

In one embodiment, the lens can be in a form of a contact lens or glasses. In one embodiment, in babies, children, etc., the glasses are held in place by an adjustable band over the eye that goes around the head and is adjusted by a hook-and-loop fastener band (i.e., a Velcro® band).

In one embodiment, the transitional lens with a central pinhole with the darkened wall or virtual pinhole and a mask is extraocular to the eye. This pinhole or virtual pinhole transitional lens may be used as contact lenses (e.g., a cosmetic, refractive, scleral lenses, or bandage lenses, etc.) or in glasses, goggles, telescopes, cameras, microscopes, etc. The technology can be used in gear or equipment for all kinds of sports-related indoor or outdoor activities including hunting, golf, tennis, swimming, etc., and in activities where goggles, etc. are used in animals, pets, racing animals, etc.

In one embodiment, the method may be applied to the telescopic lens increasing the focal point of the lens or telescope. In one embodiment, the lens is incorporated in clip-on glasses that attach to regular glasses (i.e., spectacles). In one embodiment, the clip-on glasses are themselves sunglasses. In one embodiment, the spectacles are sunglasses.

In one embodiment, the chromophore in the polymer can be loose (inside the intraocular lens (IOL) or outside) or bound to the polymer. The lens after production can be encapsulated in a non-permeable thin layer of carbon by electron spattering, plasma spattering, nanotechnology or other known methods, or other transparent molecules can be used as a transparent capsule to retain the chromophore permanently inside the IOL or other lenses and to prevent its exit. The encapsulation prevents the actual or potential chromophore removal from the lens, e.g., from leaching, diffusion, dissipation, etc., or to contain any loss that might occur.

In one embodiment, the pinhole lens with the darkened wall can be rigid or foldable. The light absorbing material can be included in the intraocular lens (IOL) material (or in the contact lens material, inlay material, glasses material, etc.), during the production of the darkened pinhole IOL or the IOL can be coated with a light absorbing material.

In one embodiment, the surface of the pinhole with the darkened wall of the intraocular lens (IOL), contact lens, inlay, or glasses can be modified to act as a light absorbing and anti-reflecting material when exposed to the external light.

In one embodiment, the light absorbing material can be cross-linked with the intraocular lens (IOL) material.

In one embodiment, the chromophore in the polymer can be loose inside the intraocular lens (IOL) with the pinhole and darkened wall, or outside, or bound to the polymer, and does not change the refractive power of the lens by radiation. The lens after production can be encapsulated in a non-permeable thin layer of carbon by electron spattering, plasma spattering, nanotechnology or other known methods or using other transparent molecules to retain the chromophore permanently inside the IOL and to prevent its exit.

In one embodiment, the intraocular lens (IOL) with pinhole and darkened wall can be implanted after a cataract extraction, or used as a contact lens with copolymers over the existing crystalline lens in patients with loss of pigment (albinism), etc., or as an additive or after loss of a part of the iris, or external to the eye, as in sunglasses.

In one embodiment, because, in the light, the chromophore can create by darkening a new dark iris around the central pinhole of the intraocular lens (IOL), inlay, contact lens, or glasses, creating a condition that the external objects become in focus not only for objects located in the far, but also for those located at near, etc., are always in focus (pinhole effect) on the retina and eliminates spherical and chromatic aberration of the IOL, which is useful for babies and encourages the eye to focus with both eyes, preventing strabismus even when the two eyes have a different dioptric power, and thereby preventing amblyopia.

In one embodiment, this concept of a darkened pinhole/transitional lenses can also be used in contact lenses, intracorneal implants, or reading glasses. Incorporating a pinhole in the intraocular lenses (IOLs), contact lenses, and glasses eliminates the darkness that would persist, when the entire lens is coated as in standard transitional lenses, it makes seeing or driving difficult. For example, when a driver moves from the sun into a tunnel (light to darkness), the standard transitional glasses stay dark for a while, which makes secing the road difficult. By contrast, in the present lens with a darkened pinhole wall in the center of a transitional lenses, the central area of the lens remains always transparent and permits the driver to see objects in a dark tunnel during the transition from light to dark.

In one embodiment, the central pinhole with the darkened wall of the present lens can have a diameter of 0.5 to 5 mm or more, e.g., in animals. More particularly, for an intraocular lens (IOL), the central pinhole has a diameter of 1 to 2 mm, while for a contact lens, the central pinhole has a diameter of 1 to 3 mm. For glasses, the central pinhole has a diameter of 1 to 4 mm or more so that the light that passes through this area of the lens is not impeded or absorbed by the chromophore or light-absorbing material.

In one embodiment, the central pinhole with the darkened wall of the present lens can have a slit with a darkened wall or oval-shaped appearance in the vertical or horizontal direction as needed (e.g., in glasses) that can be evaluated for decision making, where the diameter of the opening is 1-3 mm or more and the borders of the hole are rounded and darkened to prevent light reflection, while permitting the eye to move or see slightly to the right or left without losing the sight through the central opening.

In one embodiment, a lens having a pinhole with a darkened wall or virtual or pinhole lenses eliminates the need for progressive lenses that requires advanced expensive technology to prevent the side effect of getting dizzy that people do not tolerate well, because the pinhole with the darkened wall or virtual pinhole glasses are in focus for close up vision, such as for reading, and distance vision, which can be used by the children and adults, and eliminate presbyopia in adults, and reduce or eliminate astigmatism depending on the shape of the pinhole or the direction of a virtual slit as desired.

In one embodiment, the pinhole or slit with a darkened wall can be made where the position of the slit can be moved by rotating the glass inside its frame for the patient to test the proper location and/or the direction of the slit or oval virtual hole, or the slit can be created by a mask for each patient or each eye separately.

In one embodiment of glasses, a virtual pinhole is produced by gluing a circular or slit like or oval-shaped mask on the glasses.

In another embodiment, the central area is used for the virtual pinhole or slit is created separately from the same material used for glasses, that lacks the nanoparticles that darken by light, but has has a UV absorber to prevent glare, and the peripheral had the transitional lens; the two piece lenses are glued together with a thin mask covering the junction between the two pieces from inside or outside, or both pieces are glued together that hardens with light, a laser, exposure or by moisture or water permanently.

In one embodiment, the coating material further comprises nanoparticles. The glass frames may have nanoparticles that contain solar cells. The solar cells may be used to power electrical systems for changing the pigments color and/or charge batteries, and may be located outside the glasses. The nanoparticles can have various functional abilities, such as a sensor that detects or measures wind speed, humidity, temperature, a distance to an object positioned (e.g., by a global positioning system (GPS)), body temperature, etc.

In one embodiment, the frame may contain the chromophore-containing material, either the entire frame or one or more parts of the frame. The frame can be custom-molded and/or custom-fabricated from metallic non-metallic plastic, polymeric material for shape and/or size to minimize light from entering the eye, particularly for albinos. In one embodiment, the side frame can extend slightly beyond the plane of the iris/pupil backwards to minimize scattered light entry from the side in the eye.

In one embodiment, the top frame can extend toward the forehead. This embodiment minimizes light entry from the top, simulating a transitional awning. In other embodiments, the nose bridge, earpiece, and/or other frame portions of the glasses contain the chromophore-containing material. The frame may be fabricated such that the inventive glasses can easily be inserted into (“pop in”) and removed from (“pop out”) from the glasses frame.

In one embodiment, the full-thickness cornea is provided by an animal (e.g., pig, cow, rabbit, etc.) or human eye bank eye in which all cellular elements of the cornea including bacteria, viruses and practices are killed with riboflavin, or methylene blue, etc. and UV or other wavelength of visible or invisible light or other radiations, etc. In this embodiment, the animal corneal or human corneas may be exposed to known chemicals that damage the RNA or DNA of the animal or human corneas to prevent rejection by the host.

In one embodiment, the transplant is made from an organic material, such as collagens or other organic compounds, etc.

In one embodiment, the cornea or the corneal inlay can be made partially or entirely from a synthetic compound, such as acrylic, methacrylic, poly(methyl methacrylate) (PMMA), or other polymers that are transparent, such as polyvinylidene fluoride (PVDF), polycarbonate, etc. or a mixture of them.

In one embodiment, the corneas or the corneal inlays may be made from an organic material that can be molded and their refractive errors can be adjusted according to the refractive power that the eye needs to focus the objects not only in near, but also far and intermediate distances in these eyes that have lost their accommodative abilities because the corneas are removed and the lenses have been replaced with the standard acrylic lenses, which all have one dioptric power that has been initially chosen for the patient that at times, and has lost a proper pupil that can contract by becoming smaller by light or larger at night.

In one embodiment, the synthetic lens has two components, which include: (i) a first peripheral component that is thin and flexible and made mostly from acrylic material or silicon or in combination with or without small holes through which the tissue can grow and stabilize the artificial cornea; and (ii) a second central synthetic corneal component mostly made of acrylic methacrylate or polycarbonate, etc.

In one embodiment, the synthetic corneal inlay is molded or 3-D printed so as to permit the artificial cornea to become a desired shape during the printing. The synthetic corneal inlay is molded or 3-D printed from, for example, a very biocompatible material, such as polyvinylidene fluoride (PVDF) alone or a combination of polyvinylidene fluoride (PVDF) with silicone, etc. compound to increase its flexibility of the synthetic material and permit it to be used under the conjunctiva or inside the sclera surrounding the synthetic cornea. The peripheral potion of the keratoprosthesis can be made from polyvinylidene fluoride (PVDF) or a mixture of other polymeric compounds that renders the skirt of the inlay thin and flexible and biocompatible.

In one embodiment, the peripheral skirt has a very small <100 micron diameter tubes with or without valves that open when the intraocular pressure exceeds 10 mmg and collapse or closes when the pressure drops below 10 mmg permitting an automated control of intraocular pressure as determined prior to the implantation. The micro tubes open inside or under the conjunctiva permitting excess fluid to be absorbed by the conjunctival tissue.

In one embodiment, the retina is examined in a regular intervals using optical coherence tomography (OCT) to determine the significance of the rise of intraocular pressure by the cap/disc ratio that increases in presence of a IOP pressure that cannot be tolerated by the optic nerve head and loss of density of the nerve fiber layer of the retina or the density of the foveal capillaries all examined by OCT to indicate diminishing of the values, and as examined by artificial intelligence (AI), and all also can be photographed or examined through the transparent central synthetic lens which made with the described transparent material.

In one embodiment, the synthetic cornea may be made from acrylic or methacrylic or polyvinylidene fluoride (PVDF), and can undergo an examination to find out which refractive error that the synthetic cornea has so as to provide a 20/20 vision if the retina is not damaged.

In one embodiment, the synthetic corneas can be modified with an excimer laser to modify the outer curvature of the keratoprosthesis.

In one embodiment, the synthetic corneas can be modified with a femtosecond laser by cutting and removing a certain amount of the synthetic cornea to correct refractive error of the eye by removing the surface of the keratoprosthesis, thereby creating either a less convex surface for patients with myopia or slightly more convex surface for patients with hyperopia without affecting the intraocular pressure of the eye, etc. This procedure can corrected initially up to a 40 micron depth of the surface or less. However, the procedure can repeated if >4 dioptric power is needed to create emmetropia for the patient without influencing the intraocular pressure. In fact, most of refractive errors including astigmatic correction or higher order aberration can be corrected.

In another embodiment, the central synthetic cornea is made with any transparent polymer, but preferably using polyvinylidene fluoride and creates a bifocal or multifocal lens for the patient to see near and far at certain distances as described above, where the surface of the keratoprosthesis has different zones providing various refractive powers for the eye.

In one embodiment, the keratoprosthesis is modified to have a pinhole effect by 3-D printing the synthetic corneal from the polymeric materials described above, but preferably from polyvinylidene fluoride (PVDF) that forms the most peripheral part of the lens, while an area of 1-3 mm central area is made one zone which is dark by adding carbon black to the polyvinylidene fluoride (PVDF) to create a dark central zone where the center can be cut to make an intraocular lens with a through aperture-type pinhole having a darkened wall.

In one embodiment, the keratoprosthesis is modified to have a virtual pinhole effect by 3-D printing the synthetic corneal inlay (or lens, glasses, or contact lenses) from the polymeric materials described above, but preferably from polyvinylidene fluoride (PVDF) that forms the most peripheral part of the lens while an area of 1-2 mm of the polyvinylidene polymer is combined during the 3-D printing by one adding nanoparticles of carbon black to the polymer to create a annular zone of a darkened polymer, then the 3-D printing is continued to create a central zone of clear transparent polymeric material, such as polyvinylidene fluoride (PVDF).

FIG. 51A shows an illustrative embodiment of a 3-D printed lens 2470 with a lens body 2472 and a central circular dark portion 2474, which is cut away (as depicted in FIG. 51B), thereby leaving a circular aperture 2476 bounded by a darkened wall 2478. For example, the central circular dark portion 2474 may have a diameter of 1-2 mm, and then a central cutaway area having a diameter of 0.9-1.9 mm is removed to create a pinhole 2476 bounded by a darkened wall 2478.

FIG. 52 is a front view of a clear 3-D printed lens 2480 with a lens body 2482 having a plus or minus optical power, according to another illustrative embodiment of the invention.

FIG. 53 is a front view of a 3-D printed lens 2490 with a clear peripheral portion 2492 and a clear central pinhole 2496 bounded by a darkened peripheral wall 2494, according to still another illustrative embodiment of the invention. The clear central pinhole 2496 and clear peripheral portion 2492 may be formed from clear acrylic, methacrylic, poly(methyl methacrylate) (PMMA), and/or polycarbonate, while the darkened peripheral wall 2494 may be formed from polyvinylidene fluoride (PVDF) combined with carbon black nanoparticles or microparticles added during the 3-D printing of the lens 2490. The 3-D printed lens 2480 of FIG. 53 may be used for a corneal inlay, a scleral lens, an intraocular lens, a contact lens, or for external glasses (e.g., with transitional lens), etc.

FIG. 54 is a front view of a 3-D printed lens 2500 with a lens body 2502 made of mixed polymers, according to yet another illustrative embodiment of the invention. The 3-D printed lens 2500 of FIG. 54 may comprise mixed polymers for forming a transitional lens with one or more ring portions formed from polyvinylidene fluoride (PVDF) combined with carbon black nanoparticles or microparticles added during the 3-D printing of the lens 2500.

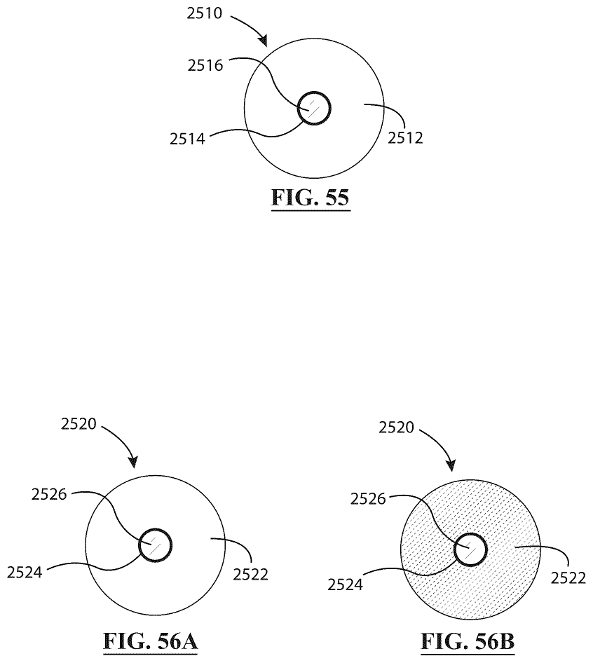

FIG. 55 is a front view of a 3-D printed lens 2510 with a peripheral portion 2512 and a clear central pinhole 2516 bounded by a darkened peripheral wall 2514, according to still another illustrative embodiment of the invention. The clear central pinhole 516 and peripheral portion 2512 may be formed from clear acrylic, methacrylic, poly(methyl methacrylate) (PMMA), and/or polycarbonate, while the darkened peripheral wall 2514 may be formed from polyvinylidene fluoride (PVDF) combined with carbon black nanoparticles or microparticles added during the 3-D printing of the lens 2510.

FIG. 56A is a front view of a 3-D printed transitional lens 2520 containing chromophores and a central pinhole 2526 passing through the lens body 2522 that is surrounded by a darkened wall 2524, according to yet another illustrative embodiment of the invention, wherein the transitional lens 2520 is depicted in a low level lighting condition where the chromophores in the lens body 2522 are not activated such that light passes through the lens body 2522 outside of the pinhole 2526. The darkened peripheral wall 2524 may be formed from polyvinylidene fluoride (PVDF) combined with carbon black nanoparticles or microparticles added during the 3-D printing of the transitional lens 2520. The 3-D printed lens 2520 of FIG. 56A may be used for a corneal inlay, a scleral lens, an intraocular lens, a contact lens, or for external glasses (e.g., with transitional lens), etc.

FIG. 56B is a front view through the 3-D printed transitional lens 2520 of FIG. 56A, wherein the transitional lens 2520 is depicted in a high level lighting condition where the chromophores in the lens body 2522 are activated such that light is prevented from passing through the lens body 2522 outside of the pinhole 2526.

In one embodiment, the synthetic corneal inlay can provide all the functionality of being able to correct refractive errors for myopia, hyperopia, and/or astigmatism, and function simultaneously as a corneal drainage system that acts automatically when the intraocular pressure (IOP) rises inside the eye.

In another embodiment, the synthetic lens may function as an intraocular lens to replace the patient's cataract with an optic that fits in the lens capsule after removal of the lens cortex with the haptics attached to the ciliary body of the eye, or the synthetic lens may function as an intraocular contact lens where the refractive power of the eye can be corrected using a femtosecond laser prior to the implantation, but the lens is endowed with a virtual pinhole as described at above for the patient to see far and near, or a combination of the pinhole with a multifocal lens.

In one embodiment, the synthetic corneal inlay may serve as a lens for an albino patient to replace glasses where the lens is made mostly form polyvinylidene fluoride (PVDF) and 3-D printed as described above, but the peripheral lens contains compound that renders the lens to act like a transitional lens in its peripheral area until in a zone of 1-3 mm so that the central circular area where a zone of the virtual lens described above is created for the patient to see through it without darkening by exposure to the light. This lens can be used for anyone who is bothered by the sunlight.

In one embodiment, in each operation involving implantation of a keratoprosthesis or intraocular lens (IOL), or 3-D printed cornea, ones inject a non-toxic dose of a Rock inhibitor, a Wnt inhibitor, an integrin inhibitor, or a GSK inhibitor in a slow release or non-slow release form with or without a non-toxic dose of antibiotics and/or low molecular weight heparin in the eye or anterior chamber after surgery to prevent inflammatory pathways of the cells, while encouraging the nerve growth or endothelial cells growth inside the eye and outside the eye.

In one embodiment, all keratoprosthesis eyes are crosslinked outside their central corneal implant with riboflavin and UV radiation applied in an oscillatory fashion, at their circular juncture of the central area and the implanted flexible peripheral skirt, to the conjunctiva and sclera creating a peripheral fibrous tissue to prevent vascular growth and inflammation as a result of a potential immune response, thereby preventing rejection of the implant.

It is obvious that many modifications can be created using the features described above with other polymers, etc., or for various applications, including cameras, telescopes, or automated improved view for recreation hunting, etc., and various form of glasses or AR/VR glasses and the system can be combined with a software in optical cameras or AR/VR glasses, etc., or as contract lenses for treating amblyopia in very young children or combined with tunable prisms in AR/VR glasses, etc. or in phoropter systems for measuring the refractive errors, etc.

In one embodiment, a 3-D printed intraocular lens with a pinhole and a darkened wall is provided. The 3-D printed intraocular lens may be in a form of a 3-D printed transitional lens with or without a transitional chromophore. Rather than being in a form of an intraocular lens, the 3-D printed lens may be in a form of contact lenses, scleral contact lens or glasses.

In one embodiment, an intraocular lens (IOL) is 3-D printed using any available material that has non-toxic biocompatibility, is hygroscopic, absorbs and holds water, and has an index of refraction of 1.4 (e.g., a polymer or polymers may be used). The intraocular lens may be formed to a desired 3-dimensional structure with or without its haptics.

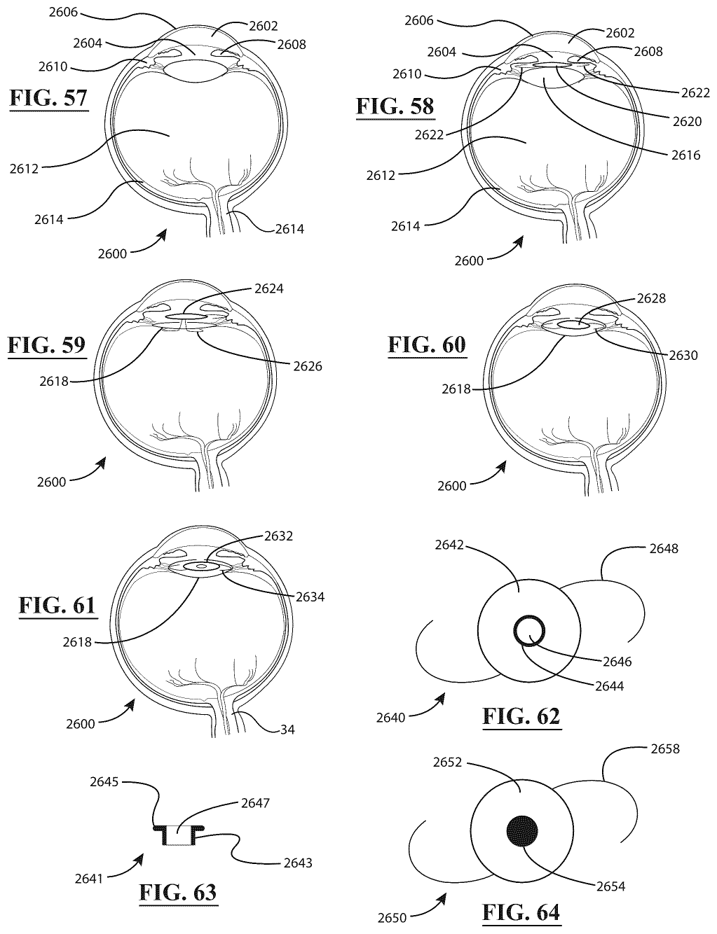

In one embodiment, an intraocular lens (IOL) is 3-D printed using any available non-toxic transparent polymer with dioptric powers of +0.1-30 D at steps of 0.1 D and dimensions of 5-8 mm in diameter and optic, and the length of 1-18 mm as combined optic and haptic diameter, etc., placed inside or outside the lens capsule, e.g., as implantation of a collamer lens (refer to FIGS. 57-60). In FIG. 57, it can be seen that an eye 2600 comprises an anterior chamber 2602, a pupil 2604, a cornea 2606, an iris 2608, a ciliary body 2610, a vitreous cavity 2612, a retina 2614, and an optic nerve 2615. In the illustrative embodiment of FIG. 58, a collamer intraocular contact lens 2620 with haptics 2622 has been implanted in the eye 2600 in front of the crystalline lens 2616. As shown in FIG. 58, the haptics 2622 support the collamer intraocular contact lens 2620 from the ciliary body 2610 of the eye 2600.

In one embodiment, transparent polymers are used to 3-D print optic and/or haptics separately or haptics attached to its optic at the desired places to give the intraocular lens (IOL) a position that is perpendicular to the light that comes from the outside of the eye passing through the cornea and the pupil. The light then reaches the retina in the back of the eye to stimulate the retinal photoreceptors of the retina to see an object.

In one embodiment, the polymers used for the haptics and optics are different from each other and the lens and haptics is either 3-D printed as one piece, or separately and combined subsequently.

In one embodiment, the 3-D printed optic is a flat plate, initially without a refractive power, then the desired dioptric power is made by ablating one or both surfaces with an excimer laser under a computer software control to a specific desired power.

In one embodiment, the polymers are made of, e.g., collamer polymer, which is a composite of one or more transparent polymers with a desired collagen component as a foldable IOL injected through a small incision made in the side of the cornea and the lens is ejected, placed behind the pupil in front of the existing crystalline lens with a vault to separate it from the crystalline lens and iris for a distance of 0.25 to 0.75 mm away from the iris and the crystalline lens. The IOL is unfolded so that its haptics reach the ciliary sulcus and stabilizes the position of the IOL. This IOL is called an intraocular contact lens (ICL), and corrects the remaining refractive power of the eye, e.g., instead of doing a laser refractive surgery on the cornea, the restriction of implantations are the corneal endothelial count less than 2,000 cells, older patients >45 years and present inflammatory processes in the eye.

In one embodiment, the IOL is made from hydrophobic acrylic, hydroxyethyl methacrylate, poly-HEMA for increased flexibility, methylmethacrylate, polymethyl methacrylate, or silicone, etc. in any form to be either a hydrophobic, hydrophilic acrylic, or hydrophilic, rigid or flexible or foldable to 3-D print each part (e.g., lens body and the haptic or haptics) from any desired shape and dimension with or without a through pinhole with a darkened wall (see FIGS. 60 and 61). In the illustrative embodiment of FIG. 60, an intraocular lens 2628 has been implanted inside the lens capsule of the eye 2600, where the haptics 2630 of the intraocular lens 2628 support the intraocular lens 2628 from the lens capsule of the eye 2600. In the illustrative embodiment of FIG. 61, an intraocular lens 2632 has been implanted inside the lens capsule of the eye 2600, where the haptics 2634 of the intraocular lens 2632 support the intraocular lens 2632 from the lens capsule of the eye 2600. Prior to insertion into the lens capsule of the eye 2600, the intraocular lens 2632 of FIG. 61 has been 3-D printed with a hole extending through the body of the intraocular lens 2632.

In one embodiment, the 3-D printed lens can have any desired characteristics, hydrophilic or hydrophobic with HEMA or in combination with silicone or collagen etc. as desired.

In one embodiment, the 3-D printed polymeric lens can have any dioptric power for minus lenses from −0.1 diopter to −40 D or more or +0.1 Diopter to +40. D or more.

In one embodiment, the lens can have any additional desired configuration to correct astigmatic, defocus or make it multifocal, monofocal, or correct higher or lower order aberrations, since the 3-D printing permits 0.1 Dioptric corrections, as compared with the present lenses that can correct the dioptric powers of only +0.25 power plus or more or toric corrections having two surfaces one spheric and the other astigmatic, like a cap.

In one embodiment, the 3-D printed lenses can have two or more haptics to reach the wall of the lens capsule or the wall of the eye (from inside the eye), e.g., the posterior chamber sulcus.

In one embodiment, the haptic is positioned behind the posterior capsule through a hole created in the posterior capsule after removal of cataractous lens cortex and nucleus, thereby leaving the anterior and posterior lens capsule.

In one embodiment, the polymeric lens with haptics, a central pinhole, and a darkened wall surrounding the central pinhole is positioned between the posterior iris and anterior lens capsule.

In one embodiment, the 3-D printed lens is located in front of the anterior capsule while the haptics are behind the posterior lens capsule (after removal of the lens cortex and nucleus) while both the anterior lens capsule collapse on each other, fixed behind the lens (refer to FIG. 59). In the illustrative embodiment of FIG. 59, an intraocular lens 2624 has been implanted in the eye 2600 in a location anterior of the lens capsule after cataract extraction, where the haptics 2626 of the intraocular lens 2624 extend behind the lens capsule.

In one embodiment, the 3-D printed lens (see FIG. 62) has a central through pinhole with a darkened wall created by administering a combination of a transparent polymer with darkening nanoparticles, e.g., carbon nanoparticles, that does not permit the light to pass through it without refracting or reflecting, reaching the retina. Therefore, the patient with a functional retina sees any object close or far or at any intermediate distance sharp in focus, where the diameter of the pinhole is between 1-3 mm with a blackened wall to prevent light scatter. In the illustrative embodiment of FIG. 62, a 3-D printed intraocular lens 2640 with haptics 2648 and a lens body 2642 comprising a central pinhole 2646 surrounded by a darkened peripheral wall 2644 is illustrated.

In one embodiment, the 3-D printed pinhole structure (refer to FIG. 63) has a through pinhole of 1-3 mm with the darkened walls of 0.1-1 mm thickness with a central through opening which can be inserted inside an IOL with a central hole of 1-3 mm in diameter, thereby creating an IOL with a central pinhole. For example, in the illustrative embodiment of FIG. 63, a 3-D printed insertable pinhole lens structure 2641 comprises a lower cylindrical body portion 2643, a top peripheral flange portion 2645 connected to the lower cylindrical body portion 2643, and a central pinhole 2647 extending through the pinhole lens structure 2641.

In one embodiment, the 3-D printed lens has a through pinhole of 1-3 mm with the darkened wall by adding a darkening element, such as carbon nanoparticles, etc., to the polymeric material during the printing of the edges of the central part of the IOL, as shown in FIG. 62.

In one embodiment, the 3-D printed lens has a through pinhole of 1-3 mm with the darkened wall so that an area of 0.1 mm to 3 mm of the wall is made with the same transparent polymers and the surrounding polymeric lens that makes the pinhole is darkened with a dark element, such as carbon nanoparticles, etc., which is added to the polymer while printing, to darken up the edges of the pinhole, while the edges of the pinhole are 3-D printed, thus creating a central through pinhole in any IOL with the desired configuration or composition, eliminating the need of having a separate polymeric pinhole, having the same flexibility for folding the lens during the its implantation in the eye for any location outside or inside the eye, from the corneal surface as a contact lens, to the cornea as an inlay, or a pinhole IOL.

In one embodiment, the lenses (e.g., intraocular lenses) are made from any biocompatible transparent polymer with the desired refractive powers. However, the central area of 1-4 mm can be made from a combination of the polymer to which a darkening compound is added to produce two zones, one central darkened area of 1-4 mm or more, and one area outside this area (refer to FIGS. 64 and 65), where a through hole can be subsequently drilled inside the darkened area (see FIG. 65), where the hole has a diameter of 1-3.7 mm with the through hole created to act as a pinhole with a darkened wall, since the pinhole is drilled with an instrument that has a diameter <4 mm, leaving a ring of dark area that forms its wall. In the illustrative embodiment of FIG. 64, a 3-D printed intraocular lens 2650 comprises haptics 2658, a central darkened zone 2654, and a peripheral clear zone 2652 surrounding the central darkened zone 2654. FIG. 65 is another front view of the 3-D printed intraocular lens 2650 of FIG. 64 after a central hole 2656 has been drilled through the central darkened zone 2654 so as to create the through pinhole.

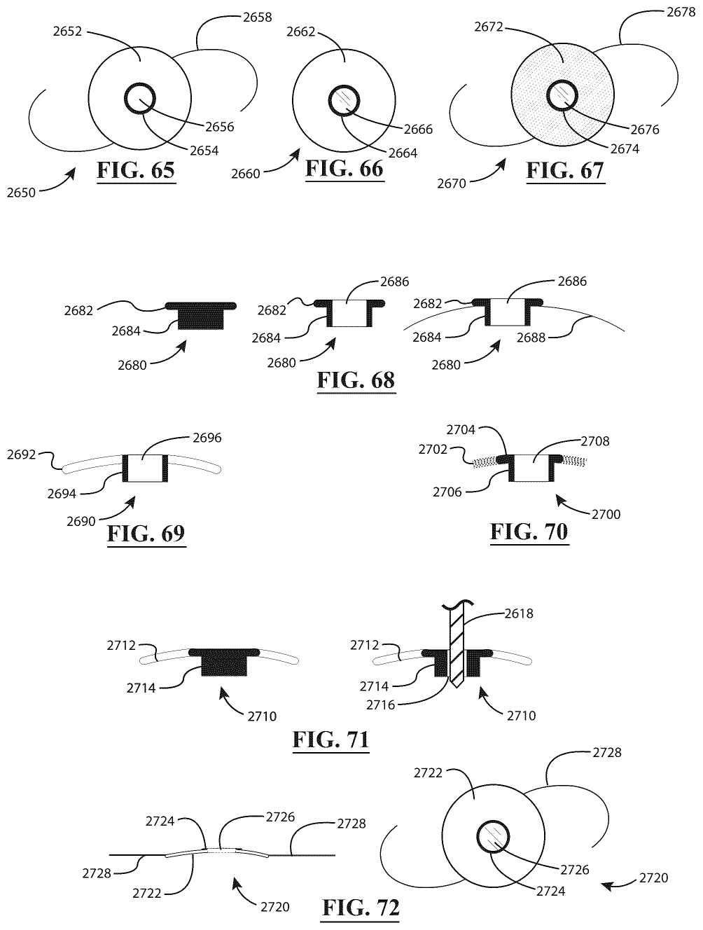

In one embodiment, the 3-D printed lens has a virtual transparent through clear central area of 1-3 mm and is made with the darkened wall where the area of 0.1 mm to 3 mm wall is made with the same polymers and the surrounding polymeric lens that makes the virtual pinhole is darkened with a dark element, such as carbon nanoparticles, etc., that are added to the polymer while printing, where its center is not a hole but a virtual transparent polymeric material, with or without a UV absorber (see FIG. 66) as a virtual hole pinhole with the dark ring eliminating the need of placing an external mask on the lens surfaces. In the illustrative embodiment of FIG. 66, a 3-D printed intraocular lens 2660 comprises a lens body 2662 with a central virtual pinhole 2666 and a darkened wall 2664 surrounding the central virtual pinhole 2666.

In one embodiment, the lens with a virtual circular darkened area is used for creating a virtual pinhole in 3-D printed lenses, such as contact lenses, scleral lenses, or glasses to be used with a glass frame, etc.

In one embodiment, the 3-D printed lens has a virtual transparent through clear central area of 1-3 mm with the darkened wall, and an area of 0.1 mm to 3 mm of the darkened wall is made with the same polymers, but with addition of carbon nanoparticles, etc., and the surrounding polymeric lens that makes the virtual pinhole is darkened with a dark element, such as carbon nanoparticles, etc. that is added to the polymer while printing, to darken up the a ring shaped area of 1-3 mm, where its center is not a hole but the same polymeric material(s) with or without a UV absorber, while creating a virtual pinhole without using a mask on the lens surfaces.

In one embodiment, one can create a virtual pinhole of 1-3 mm for 3-D printed contact lenses or glasses where the polymeric material that surrounds the virtual pinhole contains chemical compounds that darken after the light exposure, i.e., producing a transitional area (lens) outside the perimeter of the virtual pinhole that darkens with the exposure to the side light while the central virtual pinhole has only a UV absorber and does not darken up in the light (see FIG. 67), thereby creating a pinhole effect in transitional lenses for glasses, contact lenses, scleral lenses, or for an IOL as desired for anyone who is bothered by light glare, such as patients with albinism, or in babies and older children. In the illustrative embodiment of FIG. 67, a 3-D printed intraocular lens 2670 comprises haptics 2678, a central virtual pinhole 2676, a darkened wall 2674 surrounding the central virtual pinhole 2676, and a light-activated, uniformly distributed chromophore in a peripheral portion 2672 of the intraocular lens 2670 surrounding around the darkened wall 2674.

In one embodiment, the IOL with the desired polymeric component of the lens and haptic is produced by molding or 3-D printing and a central hole of 1-3 mm hole is drilled inside the IOL, then a separate darkened implant 3-D printed from one or more polymers approved by the FDA, e.g., polymethyl methacrylate (PMMA) or acrylic or collamer, etc. mixed with black carbon nanoparticles to print a structure composed of a top hat of 2-3 mm or more connected with cylindrical structure 90 degrees to the flat hat, with the diameter of 1-3 mm that fits inside the previously prepared hole in the IOL, the subsequently the darkened cylinder is drilled at its center to create a through pinhole of a desired diameter between 1-2 mm, which is implanted inside the central hole of the IOL (refer to FIG. 68). FIG. 68 depicts an illustrative embodiment of a 3-D printed insertable pinhole lens structure 2680 and the manner in which the pinhole lens structure 2680 is inserted into an intraocular lens 2688. Initially, on leftmost side of FIG. 68, it can be seen that the pinhole lens structure 2680 comprises a cylindrical body portion 2684 with a top circular flange 2682. Then, in the middle of FIG. 68, the pinhole lens structure 2680 is illustrated after a central pinhole 2686 has been drilled through the cylindrical body portion 2684 of the pinhole lens structure 2680. Finally, on rightmost side of FIG. 68, it can be seen that the pinhole lens structure 2680 has been inserted into an aperture in a lens body of an intraocular lens 2688.

In one embodiment, a separate darkened pinhole is produced by starting to 3-D print using polymers approved by the FDA for standard polymers, e.g., PMMA, acrylic, or collamer, etc., mixed with black carbon nanoparticles to form the top edge of the pinhole hat with a gray not light permeable gray area of about 10 micron at the start of 3-D printing. One can now program the rest of the pinhole black using the software to print with a combination of polyvinylidene fluoride carbon and acrylic or PMMA, etc. to create automatically the small black cylinder with no hole, where the top hat diameter builds a 2-3 mm circle and the outer wall of the short cylinder with a diameter of 1-3.50 mm and a length of 0.89 mm or more including the top hat which is implanted inside the IOL central hole. The darkened cylinder is drilled to create a through and through hole in it (see FIG. 69). In the illustrative embodiment of FIG. 69, a one-piece intraocular lens and 3-D printed pinhole lens structure 2690 comprises a lens body 2692 comprising a central pinhole 2696 surrounded by a darkened peripheral wall 2694.

In one embodiment, the pinhole is prepared before implanting it in the central hole in the IOL, or it is printed as a part of the pinhole with its surrounding transparent lens.

In one embodiment, one programs the 3-printing unit so that after starting with greying the edge of the top hat, the program continues printing the rest of the cylinder with or without its central opening (hole) in the blackened polymer with the dimension, where the central hole can be 1.4 mm or more in diameter and its outer diameter will be 2.0 mm or more, with the hat of 2.0 mm or more in diameter (refer to FIG. 70). In the illustrative embodiment of FIG. 70, a 3-D printed insertable pinhole lens structure 2700 comprises a top peripheral flange portion formed using a gray polymeric material 2702 and a darker black polymeric material 2704. The 3-D printed insertable pinhole lens structure 2700 further comprises a central pinhole 2708 surrounded by a darkened peripheral wall 2706.

In one embodiment, using 3-D printing the entire lens and its haptic is 3-D printed to the desired size and the desired refractive power. However, the central 1-3 mm area can be made from the same or another polymer having dark carbon particles to create a circular area of 1-3 mm in diameter with the darkened polymer, after finishing the 3-D printing a central area of 1-2 mm is drilled inside the darkened central polymer to create a central through hole with its remaining darkened edge (see FIG. 71). FIG. 71 depicts a 3-D printed insertable pinhole lens structure 2710 formed using a dark polymeric material where the central pinhole 2716 is subsequently drilled therein. Initially, on left side of FIG. 71, it can be seen that the pinhole lens structure 2710 comprises a lens body 2712 with a darkened central cylindrical body portion 2714. Then, on the right side of FIG. 71, the pinhole lens structure 2710 is illustrated after a central pinhole 2716 has been drilled through the darkened cylindrical body portion 2714 using a drill 2618.

In one embodiment, using 3-D printing the entire lens and its haptic is 3-D printed to the desired size and refractive power. However, the polymers that are added are made from the darkened polymers and the printing continues to create a darkened polymeric edge with its central opening in the same 3-D printing session to create a through central hole with a darkened perimeter with the desired diameter.

In one embodiment, using 3-D printing, the entire lens and its haptic is 3-D printed to the desired size and refractive power. However, the printing of the lens continues close to the center of the lens so the polymers that are added are made from the darkened polymers and the printing continues to create a darkened polymeric edge for a desired size circle, then the printing continues with the original transparent polymer with or without a UV blocker to create a virtual hole with a ring dark filter in the periphery of the central virtual “pinhole” area (see FIG. 72). In the illustrative embodiment of FIG. 72, side and front views of a 3-D printed intraocular lens 2720 with haptics 2728 are depicted. As shown in these figures, the 3-D printed intraocular lens 2720 comprises a lens body portion 2722 with a central virtual pinhole 2726 and a darkened wall 2724 surrounding the central virtual pinhole 2726.

In one embodiment using 3-D printing, the entire lens and its haptic is 3-D printed to the desired size and refractive power. However, the transparent polymer has transitional chromophores close to central area that darkens with visible light exposure and is clear in the dark creating a transitional lens only in the peripheral lens. The transitional lens may be in a form of a contact lens, scleral lens, or as sunglasses, then polymers that are added from the darkened polymers to create a darkened polymeric ring with a desired size circle of 1-3 mm or more in diameter, then 3-D printing continues with the original transparent polymer having a UV absorber to create a virtual pinhole inside the ring dark filter. This lens is not only a transitional lens, but also blocks the UV radiation in its central transparent polymer (i.e., forming a virtual pinhole-refer to FIG. 73). In the illustrative embodiment of FIG. 73, a 3-D printed intraocular lens 2730 comprises haptics 2738, a central virtual pinhole 2736, a darkened wall 2734 surrounding the central virtual pinhole 2736, and a light-activated, uniformly distributed chromophore in a peripheral portion 2732 of the intraocular lens 2730 surrounding around the darkened wall 2734.

It is clear from the descriptions of these embodiments that other lenses with different structures or filters can be created not only for the eye, but also for any camera for eliminating glare where the sunlight could create disturbing glare, e.g., for the pilots, drivers, or drones, in machine vision telescopes at sea or on dry land, and in fluidic lens, fluidic cameras or AR/VR headsets.

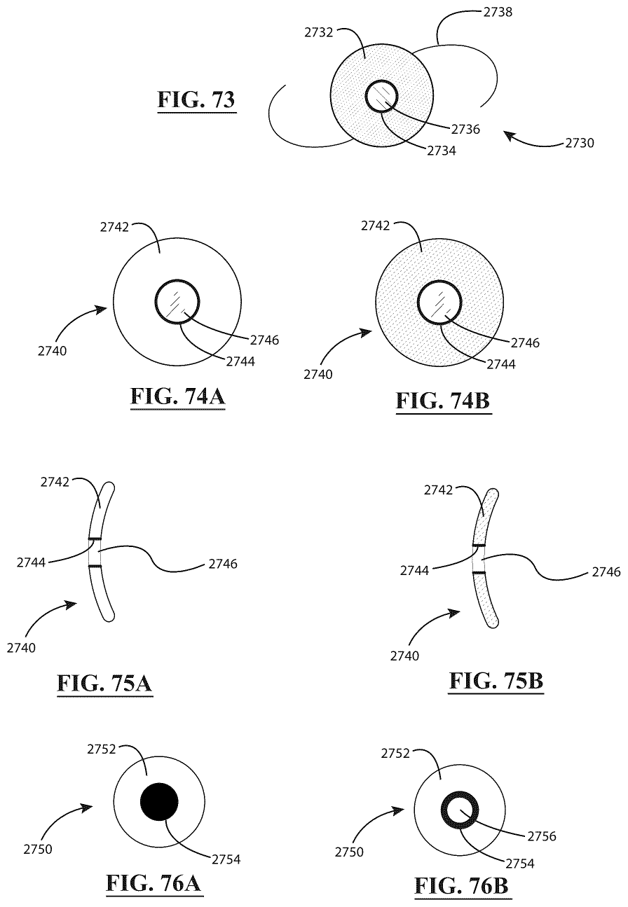

In one embodiment, the chromophore or transitional chromophore, such as silver halide and chloride embedded in the photochromic polymer, is added to the glasses or 3-D printed lens made of a transparent acrylic or poly(methyl methacrylate) or silicon or polycarbonate or transparent polyvinylidene fluoride, etc., but the chromophore addition is discontinued just before the central lens area is molded or 3-D printed, so that the center area acts like a virtual pinhole in presence of light without being light activated in its central transparent polymer (refer to FIGS. 74A and 74B). FIGS. 74A and 74B are front views of an illustrative transitional lens 2740 with a lens body having a transparent polymeric central pinhole region 2746, a peripheral transitional region 2742 with chromophores, and a darkened wall 2744 surrounding the transparent polymeric central pinhole region 2746. In FIG. 74A, the transitional lens 2740 is depicted in a low level lighting condition where the chromophores in the peripheral transitional region 2742 of the lens body are not activated such that light passes through the lens body outside of the central pinhole region 2746. In FIG. 74B, the transitional lens 2740 is depicted in a high level lighting condition where the chromophores in the peripheral transitional region 2742 of the lens body are activated such that light is prevented from passing through the lens body outside of the central pinhole region 2746.

In one embodiment, the central polymer remains clear, but a mixture of polyvinylidene fluoride with carbon black is added only around the center part of the lens to create a 1-2 mm circle or cylinder with the darkened circular wall around the clear central circular area with a thickness of 0.1-0.5 mm or more that remains clear from transitional chromophores, thus providing a clear pinhole effect for the patient through which he or she can see the object far and near in focus; since the light passing through the “pinhole” remains in focus for the retina regardless of the location of the object.

In one embodiment, in contact lenses, scleral lenses, glasses, or sunglasses, the center of the lens is made from a transparent polymers which remains clear all the time in its center but a mixture of polyvinylidene fluoride (PVF) and carbon black is added to PVF during 3-D printing to create a 1-2 or more darkened circular wall around the clear central circular area which remains free from transitional chromophores, but has transparent polymer with or without ultraviolet (UV) absorber (refer to FIGS. 75A and 75B). FIGS. 75A and 75B are side cross-sectional views of an illustrative transitional lens 2740 with a lens body having a transparent polymeric central pinhole region 2746, a peripheral transitional region 2742 with chromophores, and a darkened wall 2744 surrounding the transparent polymeric central pinhole region 2746. In FIG. 75A, the transitional lens 2740 is depicted in a low level lighting condition where the chromophores in the peripheral transitional region 2742 of the lens body are not activated such that light passes through the lens body outside of the central pinhole region 2746. In FIG. 75B, the transitional lens 2740 is depicted in a high level lighting condition where the chromophores in the peripheral transitional region 2742 of the lens body are activated such that light is prevented from passing through the lens body outside of the central pinhole region 2746.

In one embodiment, the intracorneal implant is a two-dimensional dark disc made of polyvinylidene fluoride and carbon black with a diameter of 2-3 mm where a central hole with the desired diameter of 0.3 to 1.5 mm hole or more is drilled inside the dark disc to convert it to a pinhole, where the flat two-dimensional disc has a thickness of 0.02 mm to 0.5 mm or more and can be placed inside the center of the cornea through a small incision, in front of the pupil, to create a pinhole effect for the eye, to see far or near after its implantation in the cornea alone or inside a circular organic inlay that can be shaped with a laser to a desired refractive power and implanted in the host cornea (scc FIGS. 76A and 76B). FIGS. 76A and 76B depict a transitional lens 2750 with a central darkened zone 2754 and a transitional peripheral zone 2752 surrounding the central darkened zone 2754. In FIG. 76A, the transitional lens 2750 with the central darkened zone 2754 and the transitional peripheral zone 2752 is depicted prior to the drilling of the central pinhole. In FIG. 76B, the transitional lens 2750 is illustrated after a central pinhole 2756 has been drilled through the central darkened zone 2754 of the transitional lens 2750.

Any of the features or attributes of the above-described embodiments and variations can be used in combination with any of the other features and attributes of the above described embodiments and variations as desired.

Although the invention has been shown and described with respect to a certain embodiment or embodiments, it is apparent that this invention can be embodied in many different forms and that many other modifications and variations are possible without departing from the spirit and scope of this invention. For example, a person of ordinary skill in the art will recognize various modifications are possible for humans or animals.

Moreover, while exemplary embodiments have been described herein, one of ordinary skill in the art will readily appreciate that the exemplary embodiments set forth above are merely illustrative in nature and should not be construed as to limit the claims in any manner. Rather, the scope of the invention is defined only by the appended claims and their equivalents, and not, by the preceding description.

The invention claimed is:

1. A polymeric lens, comprising:
a lens body formed from a polymeric material, the lens body comprising a central pinhole, the central pinhole being surrounded by a permanently darkened wall defining a visual axis, and the lens body being formed by 3-D printing or molding of the lens body from the polymeric material.

2. The polymeric lens according to claim 1, further comprising one or more haptics attached to the lens body, the lens body being configured for use as an intraocular lens, and the lens body being configured to be positioned between a posterior iris and an anterior lens capsule of an eye.

3. The polymeric lens according to claim 1, wherein the permanently darkened wall is made using the same polymeric material as the surrounding portion of the lens body, the permanently darkened wall being formed by adding a darkening compound to the polymeric material while 3-D printing the polymeric lens.

4. The polymeric lens according to claim 3, wherein the darkening compound used to form the permanently darkened wall in the lens body comprises carbon nanoparticles.

5. The polymeric lens according to claim 3, wherein the central pinhole of the lens body is in a form of a central virtual pinhole formed from the polymeric material without the darkening compound added thereto so as to form a central transparent virtual pinhole without the need for an external mask.

6. The polymeric lens according to claim 3, wherein the lens body with the central virtual pinhole is configured for use in glasses, as a contact lens, as a scleral lens, or as an intraocular lens that is capable of being used to significantly reduce glare for any person particularly sensitive to glare.

7. The polymeric lens according to claim 1, wherein a darkened central zone of the polymeric lens is formed by adding a darkening compound to the polymeric material while 3-D printing the lens body so as to form the lens body with the darkened central zone and a transparent peripheral zone surrounding the lens body, and wherein the darkened central zone of the lens body is subsequently drilled so as to form the central pinhole with the surrounding permanently darkened wall.

8. The polymeric lens according to claim 1, wherein the polymeric material outside the central pinhole comprises at least one light-activated, uniformly distributed chromophore that results in a darkening of the lens body outside the central pinhole when the chromophore is activated by light.

9. The polymeric lens according to claim 1, wherein the lens body with the central pinhole is configured for use in glasses, as a contact lens in an eye of a patient, as a scleral lens in an eye of a patient, as an intraocular lens in an eye of a patient, as a camera lens in a camera or fluidic camera, as a telescope lens in a machine vision telescope, in a virtual reality headset, or in an augmented reality headset.

10. A 3-D printed pinhole lens structure, comprising
an insertable pinhole body defining a through pinhole of 1-3 mm in diameter with a surrounding darkened wall having a 0.1-1 mm wall thickness, the insertable pinhole body configured to be inserted inside a lens body of an intraocular lens with a central hole of 1-3 mm in diameter, thereby creating an intraocular lens with a central pinhole.

11. The 3-D printed pinhole lens structure according to claim 10, wherein, after the insertable pinhole body is formed by 3-D printing, the through pinhole in the insertable pinhole body is formed by drilling a through hole through the insertable pinhole body.

12. The 3-D printed pinhole lens structure according to claim 10, wherein the insertable pinhole body comprises a lower cylindrical body portion with a top peripheral flange portion connected to the lower cylindrical body portion.
